(19) United States
(12) Patent Application Publication
(10) Pub. No.: US 2010/0297262 A1
(43) Pub. Date: Nov. 25, 2010

(54) PHARMACEUTICALLY ACTIVE COMPOSITIONS COMPRISING OXIDATIVE STRESS MODULATORS (OSM), NEW CHEMICAL ENTITIES, COMPOSITIONS AND USES

(75) Inventors: Hirak S. Basu, Madison, WI (US); David A. Zarling, Menlo Park, CA (US)

Correspondence Address: WILSON, SONSINI, GOODRICH & ROSATI, 650 PAGE MILL ROAD, PALO ALTO, CA, 94304-1050, US

(73) Assignee: COLBY PHARMACEUTICAL COMPANY, Menlo Park, CA (US)

(21) Appl. No.: 12/762,274

(22) Filed: Apr. 16, 2010

Related U.S. Application Data
(60) Provisional application No. 61/170,555, filed on Apr. 17, 2009.

(51) Int. Cl. A61K 33/24; A61K 31/60; A61K 31/662; A61K 31/343; A61K 31/355; A61K 31/352; A61K 31/513; A61K 31/475; A61K 31/566; A61K 31/675; A61K 31/573; A61K 31/565; A61K 31/351; A61K 31/7048; A61K 31/7072; A61K 38/09; A61K 31/519; A61P 35/00

(52) U.S. Cl. 424/649; 514/165; 514/125; 514/130; 514/468; 514/458; 514/454; 514/274; 514/285; 514/171; 424/655; 514/90; 514/459; 514/27; 514/50; 514/10.3; 514/262.1

(57) ABSTRACT

Described herein are pharmaceutical compositions and medicaments, and methods of using such pharmaceutical compositions and medicaments in the treatment of inflammation and cancer.

### CROSS-REFERENCE

This application claims the benefit of U.S. provisional application Ser. Nos. 61/170,555 filed Apr. 17, 2009, which are incorporated by reference in its entirety.

### FIELD OF THE INVENTION

Described herein are compositions that relate to Oxidative Stress Modulators (OSM), uses of various forms of oxidation/reduction (redox), nitrosative or oxidative stress-induced conditions, inflammation, hyperplasia and neoplasia, including but not limited to mammalian prostate, kidney, liver, brain, mouth, head and neck, pharanx, esophageous, stomach, colon, rectum, gonad, breast, lung, and pancreatic carcinomas and other cancers of blood and other cells, including stem cells, cancer stem cells and cells from ectoderm, endoderm and mesoderm cell origins. The compounds contain at least one or more anti-oxidant-like functional signaling moiety comprising one or more specialized quinones, hydroquinones, dihydroquinones, plastoquinones, quinols, chromanols, chromanones or certain other modified quinones, tempols, triterpenes, diamines, tetracyclenes or related functional signaling chroman-moieties. Some of these compounds are (a) with-out, or some are (b) with chemically-linked and defined-length covalently-bonded chemical linkers and some of these are (b1) with either attached nuclear-translocating compounds or alternatively some are (b2) with mitochondria-translocating compounds that comprise either (b2α) one or more quaternary cationic moieties or (b2β) one of more phytl chains of defined specific or (b2γ) a pH sensitive carbamide linkers, all with various known carbon atom lengths. Compositions and uses which modulate oxidative stress are claimed.

The present disclosure also relates to pharmaceutical compositions comprising an oxidative stress modulator (OSM) and methods for using the same. In particular, pharmaceutical compositions of the invention comprise a pharmaceutically active compound and an OSM, which reduces the in vivo oxidative degradation of the pharmaceutically active compound.

### BACKGROUND OF THE INVENTION

Typically, oxidative stress is imposed on cells as a result of one of three factors: (1) an increase in oxidant generation, (2) a decrease in antioxidant protection, and/or (3) a failure to repair oxidative damage. Cell damage is induced by reactive oxygen or nitrogen species (ROS). ROS are either free radicals, reactive anions containing oxygen atoms, or molecules containing oxygen atoms that can either produce free radicals or are chemically activated by them. Examples are hydroxyl radical, superoxide, hydrogen peroxide, peroxynitrite, etc. The main source of ROS in vivo is aerobic respiration, although ROS are also produced by peroxisomal β-oxidation of fatty acids, microsomal cytochrome P450 metabolism of xenobiotic compounds, stimulation of phagocytosis by pathogens or lipopolysaccharides, arginine metabolism, and tissue specific enzymes. Under normal conditions, ROS are cleared from the cell by the action of superoxide dismutases (SOD), catalases, or glutathione (GSH), and peroxidases. The main damage to cells results from the ROS-induced alteration of macromolecules, such as polyunsaturated fatty acids in membrane lipids, essential proteins, and DNA. Additionally, oxidative stress and ROS have been implicated in infectious and non-infectious disease states, such as inflammation, psychosis, renal disease, cardiovascular disease, diet-induced obesity and diabetes, Alzheimer's disease, Parkinson's disease, ALs, cancer, fibrosis, and aging.

Consequently, pharmaceutically active compounds (i.e., drugs) that target such diseases are subjected to in vivo oxidative or nitrosative conditions, thereby leading to degradation of at least a portion of the pro-drug or drug or a drug-related metabolite. Oxidative or nitrosative degradation effectively reduces the amount of pharmaceutically active compound that is available for chemopreventative or chemotherapeutic use leading to reduced effectiveness or a need for higher dosage to be administered, which in turn may lead to increased incidents and/or intensity of undesired side-effect(s) due to higher amount of the pharmaceutically active compound being present in vivo.

Drug metabolism is the metabolism of drugs, their biochemical modification or degradation, usually through specialized enzymatic systems. This is a form of xenobiotic metabolism. Drug metabolism often converts lipophilic chemical compounds into more readily excreted polar products. Its rate is an important determinant of the duration and intensity of the pharmacological action of drugs. Drug metabolism can result in toxication or detoxication—the activation or deactivation of the chemical. While both occur, the major metabolites of most drugs are detoxication products.

Drugs are almost all xenobiotics. Other commonly used organic chemicals are also xenobiotics, and are metabolized by the same enzymes as drugs. This provides the opportunity for drug-drug and drug-chemical interactions or reactions.

Phase I reactions usually precede Phase II, though not necessarily. During these reactions, polar bodies are either introduced or unmasked, which results in (more) polar metabolites of the original chemicals. In the case of pharmaceutical drugs, Phase I reactions can lead either to activation or inactivation of the drug. Phase I reactions (also termed nonsynthetic reactions) may occur by oxidation, reduction, hydrolysis, cyclization, and decyclization reactions. Drug oxidation involves the enzymatic addition of oxygen or removal of hydrogen, carried out by mixed function oxidases, often in the liver. These oxidative reactions typically involve a cytochrome P450 monooxygenase (often abbreviated CYP), NADPH and oxygen. The classes of pharmaceutical drugs that utilize this method for their metabolism include phenothiazines, paracetamol, and steroids. If the metabolites of phase I reactions are sufficiently polar, they may be readily excreted at this point. However, many phase I products are not eliminated rapidly and undergo a subsequent reaction in which an endogenous substrate combines with the newly incorporated functional group to form a highly polar conjugate. A common Phase I oxidation involves conversion of a C—H bond to a C—OH. This reaction sometimes converts a pharmacologically inactive compound (a prodrug) to a pharmacologically active one. By the same token, Phase I can turn a non-toxic molecule into a poisonous one (toxification). A famous example is acetonitrile, CH₃CN. Simple hydrolysis in the stomach transforms acetonitrile into acetate and ammonia, which are comparatively innocuous. But Phase I metabolism converts acetonitrile to HOCH₂CN, which rapidly dissociates into formaldehyde and hydrogen cyanide, both of which are toxic. Phase I metabolism of drug candidates can be simulated in the laboratory using non-enzyme catalysts. This example of a biomimetic reaction tends to give a mixture of products that often contains the Phase I metabolites. Phase II reactions—usually known as conjugation reactions (e.g., with glucuronic acid, sulfonates (commonly known as sulfation), glutathione or amino acids)—are usually detoxication in nature, and involve the interactions of the polar functional groups of phase I metabolites. Sites on drugs where conjugation reactions occur include carboxyl (—COOH), hydroxyl (—OH), amino (NH₂), and sulfhydryl (—SH) groups. Products of conjugation reactions have increased molecular weight and are usually inactive unlike Phase I reactions which often produce active metabolites.

Quantitatively, the smooth endoplasmic reticulum of the liver cell is the principal organ of drug metabolism, although every biological tissue has some ability to metabolize drugs. Factors responsible for the liver's contribution to drug metabolism include that it is a large organ, that it is the first organ perfused by chemicals absorbed in the gut, and that there are very high concentrations of most drug-metabolizing enzyme systems relative to other organs. If a drug is taken into the GI tract, where it enters hepatic circulation through the portal vein, it becomes well-metabolized and is said to show the first pass effect. Other sites of drug metabolism include epithelial cells of the gastrointestinal tract, lungs, kidneys, and the skin. These sites are usually responsible for localized toxicity reactions.

Several major enzymes and pathways are involved in drug metabolism, and can be divided into Phase I and Phase II reactions includes the following systems for:

### Oxidation by

Cytochrome P450 monooxygenase system
Flavin-containing monooxygenase system
Alcohol dehydrogenase and aldehyde dehydrogenase
Monoamine oxidase
Co-oxidation by peroxidases
Peroxide production from electron transport chain, metabolism, hormones, chemicals, and other signal transduction paths

### Or Reduction by:

NADPH-cytochrome P450 reductase
Reduced (ferrous) cytochrome P450

It should be noted that during reduction reactions, a chemical can enter futile cycling, in which it gains a free-radical electron, then promptly loses it to oxygen (to form a superoxide anion). Hydrolysis includes:
Esterases and amidases
Epoxide hydrolase

Factors that affect drug metabolism include the duration and intensity of pharmacological action of most lipophilic drugs are determined by the rate they are metabolized to inactive products.

The Cytochrome P450 monooxygenase system is the most important pathway in this regard. In general, anything that increases the rate of metabolism (e.g., enzyme induction) of a pharmacologically active metabolite will decrease the duration and intensity of the drug action. The opposite is also true (e.g., enzyme inhibition). Various physiological and pathological factors can also affect drug metabolism. Physiological factors that can influence drug metabolism include age, individual variation (e.g., pharmacogenetics), enterohepatic circulation, nutrition, intestinal flora, or sex differences.

In general, drugs are metabolized more slowly in fetal, neonatal and elderly humans and animals than in adults. Genetic variation (polymorphism) accounts for some of the variability in the effect of drugs. Cytochrome P450 monooxygenase system enzymes can also vary across individuals, with deficiencies occurring in 1-30% of people, depending on their ethnic background. Pathological factors can also influence drug metabolism, including liver, kidney, or heart diseases. In silico modelling and simulation methods allow drug metabolism to be predicted in virtual patient populations prior to performing clinical studies in human subjects. This can be used to identify individuals most at risk from adverse reaction

Nitrosative or Oxidative Stress has been known to contribute to a variety of human pathologies and degenerative diseases associated with aging, such as Parkinson's disease, cancers and Alzheimer's disease, as well as to Huntington's Chorea, diet-induced obesity an diabetes and Friedreich's Ataxia, and to non-specific cellular damages that accumulate with infections, inflammation and aging.

The cell nucleus and cytoplasm of some organs is a metabolic source of hydrogen peroxide, superoxide anions and hydroxyl radicals from Reactive Oxygen Species (ROS) or from Reactive Nitrogen Species (RNS). Cytoplasmic, mitochondria are the intracellular organelles primarily responsible for energy metabolism and are also a major cytoplasmic ROS source, contributing to the free radicals and reactive oxygen species (“ROS”, such as hydrogen peroxide and the superoxide radical anion (Of)) that cause oxidative stress and/or damage inside most cells. Mitochondria are equipped to detoxify hydrogen peroxide due to the presence of antioxidant enzymes (peroxiredoxins, thioredoxins, and GSH-dependent peroxidases). Typically, mitochondrial superoxide (O₂_{^{−}}., the radical anion produced by one electron reduction of O₂) is dismutated according to the stoichiometry shown below, by manganese superoxide dismutase (MnSOD) that is localized within the mitochondrial matrix.

2O₂.⁻+2H⁺→O₂+H₂O₂

However, when cellular RNS or ROS production exceeds the cell's detoxification capacity, oxidative damage can occur. This damage disrupts mitochondrial function and oxidative phosphorylation and leads to significant cellular damage to mitochondrial, other cytoplasmic or nuclear cellular proteins, DNA, RNA and phospholipids and thus induces cell damage, oxidation, inflammation, hyperplasia, neoplasia, disease and/or death. Superoxide can also react with nitric oxide at a diffusion-controlled reaction rate, forming highly potent oxidants, such as peroxynitrite and peroxynitriles, that can modify proteins and DNA through oxidation and nitration reactions. In addition to these damaging and pathological roles, ROS also act as a redox signaling molecule(s) and promotes acute inflammation, cell proliferation, DNA damage repair, genetic errors and mutation leading to chronic inflammation, hyperplasia, or neoplasia and malignancy or other disease.

Naturally occurring exogenous and endogenous tissue reactive oxygen or nitrogen species (ROS) are known to play a major role in prostate, colorectal, lymphoma and pancreatic carcinogenesis. ROS alters the activity of thiol-dependent enzymes, changes the cellular redox balance and covalently modifies proteins and modifies and mutagenizes DNA. It has also been shown that increased lipid peroxidation and production of unregulated ROS in men with high fat diets is one of the major reasons for the higher incidence of prostate cancer in industrialized nations, as compared to that in developing countries. In recent years, direct experimental evidence has linked increased ROS production with the corresponding increase in mutations and tumor development in various tissues, including in the pancreas and the prostate organs. For example, Oberley and colleagues monitored oxidative stress induced enzymes and oxidative damage to DNA bases of malignant and normal human prostate tissues. Malignant prostate tumor tissues showed significantly higher oxidative stress and ROS-induced DNA modifications compared to normal prostate tissues. Ho and coworkers (Tam et al., Prostate. 2006 Jan. 1; 66(1):57-69) demonstrated the presence of high oxidative stress induced DNA modifications in the pre-neoplastic lesions occurring in the well-studied TRAMP (Transgenic Adenocarcinoma of Mouse Prostate) prostate cancer mouse model of human prostate cancer.

Accordingly, there remains a need for nuclear or cytoplasmic-extra-mitochondrially or cytoplasmic-mitochondrially-targeted anti-oxidant or similar modulator drugs with anti-inflammatory, anti-proliferative, anti-hyperplastic, anti-degenerative, and/or anti-cancer agents as proprietary drugs or pro-drugs with improved pharmacological properties and/or toxicity profiles. It is towards the provision of such molecules, which may or may not be targeted to mitochondria, that the various inventions disclosed and described below are directed.

To function in animal or human drug therapies, cytoplasmic-delivery and extra-mitochondria-targeted or mitochondria-targeted molecules must be delivered within cells in patients, preferably following oral administration. For extra-mitochondrial targeting, the Ligand Binding Domain (LBD) of the Androgen Receptor (AR-LBD) is a membrane or cytoplasmic protein which is transferred into the nucleus. Table I describes certain known cellular systems, treatments, targeted test compounds and system outcomes.

TABLE I
Known Targeted Cellular Systems, Treatments, Targeted Test Compounds and
System Outcomes

| System | Treatment | Compound | Outcome |
|---|---|---|---|
| Mitochondrial | Peroxynitrite | MitoQ-C10 | Decreases lipid peroxidation |
| membranes | | | |
| Isolated liver | Ferrous iron/ascorbate | MitoVE-C2 | Decreases lipid peroxidation, |
| mitochondria | | | protein carbonyl formation |
| | | | and loss of membrane |
| | | | potential |
| Isolated liver | Ferrous iron/ascorbate | MitoQ-C10 | Decreases lipid peroxidation |
| mitochondria | | | and loss of membrane |
| | | | potential |
| Isolated liver | Ferrous iron/H₂O₂ | MitoQ-C10 | Decreases lipid peroxidation |
| mitochondria | | | |
| Isolated kidney | Superoxide | MitoQ-C10 | Blocks activation of |
| mitochondria | | or MitoVE- | uncoupling proteins |
| | | C2 | |
| Jurkat cells | H₂O₂ | MitoQ-C10 | Decreases apoptosis |
| Jurkat cells | H₂O₂ or α-tocopheryl | MitoQ-C10 | Decreases apoptosis |
| | succinate | | |
| Jurkat cells | H₂O₂ | MitoVE-C2 | Decreases apoptosis |
| Human umbilical vein | H₂O₂ | MitoQ-C10 | Decreases apoptosis |
| endothelial cells | | | |
| Porcine aorta | Hypoxia | MitoQ-C10 | Decreases |
| endothelial cells | | | dichlorofluorescein |
| | | | fluorescence, protein |
| | | | phosphorylation and cell |
| | | | proliferation |
| Bovine aortic | H₂O₂ | MitoQ-C10 | Decreases growth factor |
| endothelial cells | | | receptor phosphorylation |
| Bovine aortic | H₂O₂ or | MitoQ-C10 | Decreases complex I and |
| endothelial cells | hydroperoxyoctadecadienoic | or MitoVE- | aconitase inhibition, |
| | acid | C2 | apoptosis, |
| | | | dichlorofluorescein |
| | | | fluorescence. Decreases |
| | | | transferrin receptor |
| | | | expression and iron uptake. |
| | | | Preserves mitochondrial and |
| | | | proteosomal function. |
| MRC-5 fibroblasts | Hyperoxia | MitoQ-C10 | Decreases |
| | | | dichlorofluorescein |
| | | | fluorescence, and telomere |
| | | | shortening, and increases |
| | | | replicative lifespan |
| Normal Human | Partial inhibition of complex I | MitoQ-C10 | Decreases mitochondrial |
| primary skin | | | lipid peroxidation and |
| fibroblasts | | | mitochondrial outgrowth |
| Friedreich's ataxia | Glutathione depletion | MitoQ₁₀ or | Decreases cell death |
| patient primary skin | | MitoVE-C2 | |
| fibroblasts | | | |
| Normal Retinal | Blue light | MitoQ-C10 | Decreases dihydroethidium |
| pigmented epithelial | | | oxidation and cell death |
| cell line (ARPE-19) | | | |
| COS-7 cells | H₂O₂ | MitoQ-C10 | Decreases growth factor |
| | | or MitoVE- | receptor and kinase |
| | | C2 | phosphorylation |
| Rat C6 glioma cell | Manganese chloride | MitoQ-C10 | Decreases |
| line | | | dichlorofluorescein |
| | | | fluorescence, and the |
| | | | enhancement by MnCl2 of |
| | | | lipopolysaccharide activation |
| | | | of NF-κB and iNOS |
| | | | expression |
| Human | Hypoxia | MitoQ-C10 | Decreases stabilization of |
| hepatoblastoma | | | hypoxia-inducible factor-1α |
| (Hep3B) and | | | and dichlorofluorescein |
| fibrosarcoma | | | fluorescence |
| (HT1080) cell lines | | | |
| Rat | Serum withdrawal | MitoQ-C10 | Decreases apoptosis |
| pheochromocytoma | | | |
| (PC12) cell line | | | |
| Mouse NIH/3T3 | Inducible expression of | MitoQ-C10 | Prevents induction of |
| normal mouse cell | exogenous human Mn- | | endogenous Mn-superoxide |
| line | superoxide dismutase | | dismutase and thioredoxin-2, |
| | | | and blocks cell growth |
| Primary rat | Serotonin | MitoQ-C10 | Prevents hypertrophy and |
| cardiomyocytes | | | protein phosphorylation |
| Mouse N2O2 and | α-tocopheryl succinate | MitoQ-C10 | Decreases apoptosis |
| NeuD12 cell lines | | | |
| Embryonic rat heart | Doxorubicin (adriamycin) or | MitoQ-C10 | Decreases apoptosis, caspase |
| cell line (H9c2) | H₂O₂ | | activation, |
| | | | dichlorofluorescein |
| | | | fluorescence and nuclear |
| | | | translocation of NFAT |
| | | | (nuclear factor of activated T |
| | | | lymphocytes) |
| Mouse colonocyte cell | Docosahexaenoic acid and | MitoQ-C10 | Decreases lipid peroxidation |
| line (YAMC) | butyrate | | and apoptosis |
| Rat primary cerebellar | Ethanol | MitoVE-C2 | Decreases |
| granule cells | | | dichlorofluorescein |
| | | | fluorescence and cell death |
| Mouse pancreatic | Cholecystokinin | MitoQ-C10 | Decreases hydroethidium |
| acinar cells | | | oxidation and calcium |
| | | | oscillations |
| HEK293 cells and rat | Lysophosphatidylcholine | MitoQ-C10 | Decreases the activation of |
| cardiomyocytes | | | L-type calcium currents |
| HeLa cells | H₂O₂ produced by tumor | MitoQ-C10 | Decreases death of cells |
| | necrosis factor-treated cells | | close to tumor necrosis |
| | | | factor-treated cells |
| Human colon cancer | 5-Fluorouracil | MitoQ-C10 | Decreases apoptosis from 5- |
| cell lines (HCT116 | | or MitoVE- | FU chemotherapy |
| and RKO) | | C2 | |

### SUMMARY OF THE INVENTION

One aspect of the disclosure relates to compositions and methods for treating or inhibiting the occurrence, recurrence, of a disease, inflammation, degeneration, necrosis, hyperplasia or neoplasia, including infectious or non-infectious or progressive disease or metastatic progression or metastasis, of a cancer or a disease precursor thereof, consisting of administering to a mammal diagnosed as having an inflammation, hyperplasia, neoplasia, disease or precursor disorder thereof, in an amount effective to treat or inhibit the occurrence, recurrence, progression of the inflammation, enlargement, hyperplasia, neoplasia, disease or precursor thereof, with a combination of an anti-oxidant and compounds able to undergo oxidation, for example, inhibitors of HDAC, Histone DeACetylase or other anti-cancer drugs like, Doxirubicin or Etoposide or other drugs.

In one embodiment is a method of treating cancer comprising administration of a combination comprising an HDAC inhibitor and an anti-oxidant. In another embodiment is the method wherein the cancer is an HDAC or other inhibitor resistant cancer or other disease. In another embodiment is the method wherein the cancer is selected from prostate cancer or colorectal cancer. In another embodiment is the method wherein the cancer is an androgen-responsive cancer, live Prostate Adenocarcinoma or Hapatocellular Carcinoma. In another embodiment is the method wherein the cancer is characterized by an increased level of reactive oxygen species. In another embodiment is the method wherein the cancer is characterized by an elevated level of oxidative stress, for example from increased rates of production of superoxide and/or hydrogen peroxide by cells. In another embodiment is the method wherein the HDAC inhibitor is selected from suberolylanilide hydroxamic acid, trichostatin A, trapoxin B, phenylbutyrate, valproic acid, Belinostat/PXD101, MS275, LAQ824/LBH589, CI994, and MGCD0103. In another embodiment is the method wherein the HDAC inhibitor is selected from suberolylanilide hydroxamic acid.

In another embodiment is the method wherein the anti-oxidant is selected from Vitamin E or a Vitamin E analog. In another embodiment is the method wherein the anti-oxidant is selected from a Vitamin E pro-drug, a Plastoquinone pro-drug or a Nitroxide pro-drug. In a further embodiment is a method wherein the anti-oxidant is a compound of Formula (I). In another embodiment is the method wherein the anti-oxidant is administered first. In another embodiment is the method wherein the Vitamin E is administered first.

Also described herein are pharmaceutical compositions comprising an anti-oxidant and a compound capable of undergoing oxidation. In one embodiment, the compound capable of undergoing oxidation is an inhibitor of HDAC. In one embodiment, the compound capable of undergoing oxidation is a pharmaceutical composition comprising a combination of an HDAC inhibitor and an anti-oxidant drug. In another embodiment is the method wherein the HDAC inhibitor is selected from suberolylanilide hydroxamic acid, trichostatin A, trapoxin B, phenylbutyrate, valproic acid, Belinostat/PXD101, MS275, LAQ824/LBH589, CI994, and MGCD0103. In another embodiment is the method wherein the HDAC inhibitor is selected from suberolylanilide hydroxamic acid. In another embodiment is the method wherein the anti-oxidant is selected from Vitamin E or a Vitamin E analog, a Plastoquinone or a Plastquinone analog, a Tempol or Tempol analog, or a Triterpene or a Triterpene analog. In another embodiment is the method wherein the anti-oxidant is selected from Vitamin E or Vitamin E analogs formulated as drugs or pro-drugs. In a further embodiment, the anti-oxidant is a compound of Formula (I). In another embodiment is the method wherein the composition is contained with a single unit dosage.

In one embodiment is a method of treating cancer comprising administration of a combination containing an anti-cancer agent and an anti-oxidant. In another embodiment is the method wherein the anti-cancer agent can be oxidized by a reactive oxygen species. In another embodiment is the method wherein the anti-cancer agent is selected from docetaxol, 5-fluorouracil, vinblastine sulfate, estramustine phosphate, suramin, strontium-89, buserelin, chlorotranisene, chromic phosphate, etoposide (VP16), cisplatin, satraplatin, cyclophosphamide, dexamethasone, doxorubicin, testosterone and analogs, steroids and analogs, non-steroidal anti-inflammatory drugs, including aspirin, estradiol, estradiol valerate, estrogens conjugated and esterified, estrone, ethinyl estradiol, floxuridine, goserelin, hydroxyurea, melphalan, methotrexate, mitomycin, prednisone, suberolylanilide hydroxamic acid, trichostatin A, trapoxin B, phenylbutyrate, valproic acid, Belinostat/PXD101, MS275, LAQ824/LBH589, CI994, and MGCD0103.

In another embodiment is the method wherein the anti-oxidant has the structure of Formula (I)

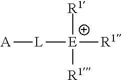

wherein:
i) A is at least one group capable of functioning as an anti-oxidant or reduced anti-oxidant, comprising a hydroquinone, dihydroquinone, quinone, plastoquinone, quinol, phenol, diamine, triterpene, tetracycline, chromanol, chromanone, chroman, tempol, tempol-H or the pro-drugs thereof, having from 2 to 30 carbon atoms;
ii) L is a linking group comprising from 0 to 50 carbon atoms; which may or may not have a pH sensitive carbodiamide linker
iii) E is no atom or a nitrogen or phosphorous;
iv) R^{1′}, R^{1″}, and R^{1′″} are each independently chosen from organic radicals comprising from 0 to 12 carbon atoms; and
b) at least one anion having the formula X^{⊖} wherein the cation and the anion, if present, are present in an amount sufficient to form a neutral, pharmaceutically acceptable salt.

In another embodiment is the method wherein the A group has the formula:

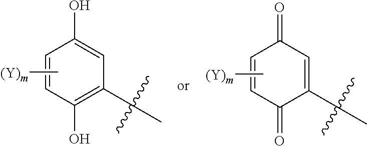

wherein Y is optionally present, and can be one or more electron activating moieties chosen from:
i) C₁-C₄ linear, branched, or cyclic alkyl;
ii) C₁-C₄ linear, branched, or cyclic haloalkyl;
iii) C₁-C₄ linear, branched, or cyclic alkoxy;
iv) C₁-C₄ linear, branched, or cyclic haloalkoxy; or
v) —N(R²)₂, each R² is independently hydrogen or C₁-C₄ linear or branched alkyl; and m indicates the number of Y units present and the value of m is from 0 to 3.

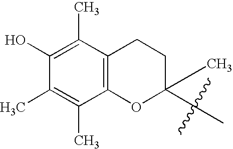

In another embodiment is the method wherein A is. In another embodiment is the method wherein the anti-oxidant is vitamin E or a vitamin E analog

In another embodiment is the method wherein the anti-cancer agent is an HDAC inhibitor. In another embodiment is the method wherein the HDAC inhibitor is suberolylanilide hydroxamic acid.

In one embodiment is a pharmaceutical composition comprising a combination of an anti-cancer agent and an anti-oxidant. In another embodiment is the pharmaceutical composition wherein the anti-cancer agent can be oxidized by a reactive oxygen species. In another embodiment is the pharmaceutical composition wherein the anti-cancer agent is selected from docetaxol, 5-fluorouracil, vinblastine sulfate, estramustine phosphate, suramin, strontium-89, buserelin, chlorotranisene, chromic phosphate, cisplatin, satraplatin, cyclophosphamide, dexamethasone, doxorubicin etoposide, steroid, estradiol, estradiol valerate, estrogens conjugated and esterified, estrone, ethinyl estradiol, floxuridine, goserelin, hydroxyurea, melphalan, methotrexate, mitomycin, prednisone, suberolylanilide hydroxamic acid, trichostatin A, trapoxin B, phenylbutyrate, valproic acid, Belinostat/PXD101, MS275, LAQ824/LBH589, CI994, and MGCD0103.

In another embodiment is the pharmaceutical composition wherein the anti-oxidant has the structure of Formula (I)

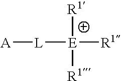

wherein:
i) A is at least one group capable of functioning as an anti-oxidant or reduced anti-oxidant, comprising a hydroquinone, dihydroquinone, quinone, plastoquinone, quinol, phenol, diamine, triterpene, tetracycline, chromanol, chromanone, chroman, tempol, tempol-H or a pro-drug thereof, having from 2 to 30 carbon atoms;
ii) L is a linking group comprising from 0 to 50 carbon atoms;
iii) E is no atom or a nitrogen or phosphorous;
iv) R^{1′}, R^{1″}, and R^{1′″} are each independently chosen from organic radicals comprising from 0 to 12 carbon atoms; and
b) at least one anion having the formula X^{⊖} wherein the cation and the anion, if present, are present in an amount sufficient to form a neutral, pharmaceutically acceptable salt.

In another embodiment is the pharmaceutical composition wherein the A group has the formula:

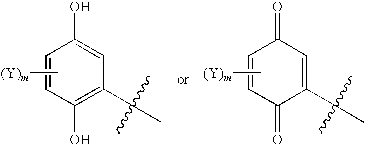

wherein Y is optionally present, and can be one or more electron activating moieties chosen from:
i) C₁-C₄ linear, branched, or cyclic alkyl;
ii) C₁-C₄ linear, branched, or cyclic haloalkyl;
iii) C₁-C₄ linear, branched, or cyclic alkoxy;
iv) C₁-C₄ linear, branched, or cyclic haloalkoxy; or
v) —N(R²)₂, each R² is independently hydrogen or C₁-C₄ linear or branched alkyl; and m indicates the number of Y units present and the value of m is from 0 to 3.

In another embodiment is the pharmaceutical composition wherein A is

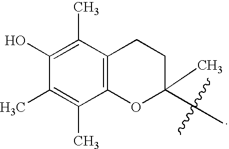

In another embodiment is the pharmaceutical composition wherein the anti-oxidant is vitamin E or a vitamin E analog.

In another embodiment is the pharmaceutical composition wherein the anti-cancer agent is an HDAC inhibitor. In another embodiment is the pharmaceutical composition wherein the HDAC inhibitor is suberolylanilide hydroxamic acid.

It is understood that the examples and embodiments described above are for illustrative purposes only and that various modifications or changes in light thereof will be suggested to persons skilled in the art and are to be included within the spirit and purview of this application and scope of the appended claims.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 shows the inhibitory effect of varying concentrations of MitoQ-C10 on the proliferation and growth of human prostate tumor LNCaP cells, as determined by Hoechst dye-DNA fluorescence assays.

FIG. 2 shows the inhibitory effect of varying concentrations of Mito-Q on the proliferation and growth of androgen independent PC-3 cells, as determined by Hoechst dye-DNA fluorescence assays.

FIG. 3 shows the inhibitory effect of treatment with varying concentrations of Mito-Q-C10 on the growth of LNCaP human prostate tumor cells as determined by the ratio of DCF fluorescence/Hoechst dye-DNA fluorescence.

FIG. 4 shows the inhibitory effect of treatment with varying concentrations of Mito-Q on the oxidative stress in LNCaP human prostate tumor cells as determined by the ratio of DCF fluorescence/Hoechst dye-DNA fluorescence.

FIG. 5 shows that synthetic androgen (metribolone) treatment-induced oxidative stress in LNCaP human prostate cancer cells determined by the ratio of DCF fluorescence/DNA fluorescence, is completely abrogated by pre-treatment of the cells with 10 nM Mito-Q.

FIG. 6 shows the intracellular levels of Mito-Q in LNCaP cells as determined by LC-MS and its correlation to cell growth.

FIG. 7 shows (a) relative DNA-Hoechst dye fluorescence as a measure of cell growth in SAHA treated LNCaP cells expressed as percent of DNA fluorescence in cells not treated with SAHA is plotted against SAHA concentration in (A) cells treated with no R1881; (B) cells treated with 0.05 nM R1881; and (C) cells treated with 2 nM R1881; and (b) cellular ROS levels measured as a ratio of DCF fluorescence:DNA fluorescence are plotted vs. SAHA concentration in (A) cells treated with no R1881; (B) cells treated with 0.05 nM R1881; and (C) cells treated with 2 nM R1881.

FIG. 8 shows cellular ROS levels measured as a ratio of DCF fluorescence:DNA fluorescence in LNCaP and PC-3 cells and LNCaP cells treated with 1 nM R1881 with ( [symbol] ) or without ( [symbol] ) pretreatment with 20 μM Vitamin E.

FIG. 9 shows growth inhibitory effect of SAHA with () without (▪) pretreatment with a previously optimized non-toxic concentration of Vitamin E expressed as DNA fluorescence percent of corresponding SAHA untreated cells plotted against SAHA concentrations in (A) LNCaP prostate cancer cells growing without androgen with or without 20 μM Vitamin E, (B) LNCaP cells growing in the presence of 1 nM R1881 with or without 20 μM Vitamin E, (C) PC-3 prostate cancer cells with or without 20 μM Vitamin E, and (D) HT-29 colorectal cancer cells with or without 6 μM Vitamin E.

FIG. 10 shows representative western blot of acetyl histone H4 (Ac-histone H4) and corresponding β-actin protein from: LNCaP cells treated with 20 μM Vitamin E (Lane #1), LNCaP cells treated with 2 μM HDAC inhibitor drug (Lane #2), LNCaP cells treated with 1 nM Androgen (Lane #3), LNCaP cells treated with 1 nM Androgen and 2 μM HDAC inhibitor drug (Lane #4), and LNCaP cells treated with 1 nM Androgen, 20 μM Vitamin E and 2 μM HDAC inhibitor drug (Lane #5).

FIG. 11 shows intracellular SAHA levels in LNCaP cells treated with 2 μM SAHA ( [symbol] ) pretreated with 1 nM R1881 followed by 2 μM SAHA ( [symbol] ) or treated with 20 μM Vitamin E+1 nM R1881 followed by 2 μM SAHA ( [symbol] ) as determined by LC-MS method and calculated from a SAHA standard curve determined from SAHA spiked medium.

### DETAILED DESCRIPTION

A popular model of early stage human prostate cancer (CaP or PCa are used interchangeably throughout) is the LNCaP cell line. It is an androgen-responsive human CaP cell line that was established from a metastatic lesion in the left supraclavicular lymph node. In culture, LNCaP cells can be treated with different levels of androgen analog metribolone to mimic serum androgen conditions of patients who have or have not undergone androgen deprivation therapy (ADT). In 1997, Ripple et al first reported that in LNCaP cells, treatment with metribolone generates varying levels of reactive oxygen species (ROS) such as superoxide, hydroxyl radical, hydrogen peroxide, etc. as determined by DCFH-DA dye oxidation assay. When treated with metribolone concentrations less than 1 nM, “low androgen”, LNCaP cells showed significantly lower cellular ROS as compared to treatment with 1 nM to 10 nM metribolone (R1881 synthetic androgen), “normal to high androgen”. However, within the 1-10 nM range, no significant difference was observed in the amount of cellular growth or ROS generated by the metribolone treatment.

The chromatin structure of DNA consists of many nucleosomes linked together by the DNA double strands. Four pairs of histone proteins are surrounded by DNA to form the nucleosomes. These histones help regulate gene transcription during cell proliferation by condensing the chromatin structure. Each histone can be modified by acetylation. As the chromatin structure condenses, the frequency of gene transcription decreases. It is known that histone deacetylase (HDAC) is a class of enzymes present mostly in the nucleus that de-acetylates histones H3 and H4. This enzymatic activity prevents the transcription of the genes required for arrest of the cell cycle. When HDAC is inhibited, arrest of cell proliferation, cell death and/or differentiation of cancer cells may occur due to expression of specific genes. Suberolylanilide Hydroxamic Acid (SAHA) is a HDAC inhibitor that causes arrest of cell proliferation and cell death. It is approved for the treatment for cutaneous T-cell lymphoma (CTCL) and also functions in lung cancer and certain other lymphomas. Other HDAC inhibitors include: Trichostatin A, trapoxin B, phenylbutyrate, valproic acid, Belinostat/PXD101, MS275, LAQ824/LBH589, CI994, and MGCD0103.

Although SAHA (Suberoylanilide hydroxamic acid, Vorinostat) has been successful in the treatment of Cutaneous T-cell lymphoma, it is not clinically effective as a solo therapy in the treatment of CaP, Colorectal, Breast and certain other types of cancers. There can be several reasons for CaP and other human tumors' resistances to certain known Chemotherapeutic drugs and HDAC inhibitors, including SAHA, e.g., (i) Compared to Cutaneous T-cell lymphoma cells, CaP and colorectal cells have higher oxidative stress and, therefore, may be immune to drugs that can induce cell kill by inducing oxidative stress, (ii) high SOD enzyme activity in CaP or other human tumors cells may neutralize oxidative stress produced by SAHA, (iii) SAHA may be oxidized under high Androgen concentration conditions by the high levels of ROS produced in the prostate and thereby, require high SAHA drug concentrations to kill prostate cells that is not clinically achievable. We demonstrate the inactivity of certain drugs, including SAHA against CaP cells with high ROS is not due to changes in SOD activity and resistance to ROS, but loss of the oxidized drug or oxidized SAHA in cells with high level of ROS. We discovered that reduction of ROS levels by silencing a major enzyme in ROS producing pathway or by pretreatment with Vitamin E or Vitamin E analogs activates SAHA against CaP cells.

We discovered that intracellular oxidative stress reduces the cytotoxicity of oxidized SAHA or other SOC cancer drugs. Certain HDAC inhibitor drugs, including SAHA, are inactive against oxidatively stressed human breast and colon cancer cells. It is also inactive against a human prostate cancers, when the tumor cells are at a high oxidative stress level. SAHA, however, markedly inhibits growth of the same human prostate cancer cell line or primary tumor, when it is at a low oxidative stress level. We also discovered that a reduction of cellular oxidative stress by pre-treatment with certain anti-oxidants synergistically sensitizes the prostate, colon and breast cancer cells with high oxidative stress to the growth inhibitory effects of SAHA or other oxidation sensitive anti-cancer drugs. Anti-oxidant water soluble chromanols, highly lipophilic ATCol (alpha tocopherol) and their analogs or other Oxidative Stress Modulator (OSM) drugs in anti-oxidant pretreatment or co-treatment protocols, however, did not sensitize human cancer cells and primary tumors that are at a low oxidative stress level.

These data directly show that it can be therapeutically important to add Chromanol-based lipid soluble or lipophilic Vitamin E or water soluble analogs in combination with certain oxidation-sensitive anti-cancer drugs. This is including combinations with SAHA for the treatment of human prostate, breast, colon and other cancers with high oxidative stress that are generally unresponsive to oxidation-sensitive drugs like SAHA or certain other oxidation sensitive HDAC inhibitors or certain other chemotherapeutic drugs that are inactive by oxidation.

### DEFINITIONS

Before the disclosure is described in detail, it is understood that the scope of this disclosure is not limited to the particular methodology, protocols, cell lines, and reagents described, as these may vary. It is also to be understood that the terminology used herein is for the purpose of describing particular embodiments only and is not intended to limit the scope of the disclosure, which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Often, ranges are expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or cannot be substituted and that the description includes both unsubstituted lower alkyl and lower alkyl where there is substitution.

A cell can be in vitro. Alternatively, a cell can be in vivo and can be found in a subject. A “cell” can be a cell from any organism including, but not limited to, a bacterium or a mammalian cell.

As used throughout, by a “subject” is meant an individual. Thus, the “subject” can include domesticated animals, such as cats, dogs, etc., livestock (e.g., cattle, horses, pigs, sheep, goats, rabbits, etc.), laboratory animals (e.g., mouse, rabbit, rat, guinea pig, ferret, mink, etc.) and birds. In one aspect, the subject is a higher mammal such as a primate or a human.

In one aspect, the compounds described herein can be administered to a subject comprising a human or an animal including, but not limited to, a primate, murine, canine, feline, equine, bovine, porcine, caprine or ovine species and the like, that is in need of alleviation or amelioration from a recognized medical condition.

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “moiety” defines a carbon containing residue, i.e. a moiety comprising at least one carbon atom, and includes but is not limited to the carbon-containing groups defined hereinabove. Organic moieties can contain various heteroatoms, or be bonded to another molecule through a heteroatom, including oxygen, nitrogen, sulfur, phosphorus, or the like. Examples of organic moieties include but are not limited alkyl or substituted alkyls, alkoxy or substituted alkoxy, mono or di-substituted amino, amide groups, etc. Organic moieties can preferably comprise 1 to 21 carbon atoms, 1 to 18 carbon atoms, 1 to 15, carbon atoms, 1 to 12 carbon atoms, 1 to 8 carbon atoms, or 1 to 4 carbon atoms.

Unless defined otherwise, all technical and scientific terms used herein have the same meanings as commonly understood by one of ordinary skill in the art. Although any methods and materials similar or equivalent to those described herein can be used in the practice or testing of the present disclosure, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the embodiments described herein.

The term “alkyl” denotes a moiety containing a saturated, straight or branched hydrocarbon residue having from 1 to 18 carbons, or preferably 4 to 14 carbons, 5 to 13 carbons, or 6 to 10 carbons. An alkyl is structurally similar to a non-cyclic alkane compound modified by the removal of one hydrogen from the non-cyclic alkane and the substitution, therefore, with a non-hydrogen group or moiety. Alkyl moieties can be branched or unbranched. Lower alkyl moieties have 1 to 4 carbon atoms. Examples of alkyl moieties include methyl, ethyl, n-propyl, iso-propyl, n-butyl, sec-butyl, t-butyl, amyl, t-amyl, n-pentyl and the like.

The term “substituted alkyl” denotes an alkyl moiety analogous to the above definition that is substituted with one or more organic or inorganic substituent moieties. In some embodiments, 1 or 2 organic or inorganic substituent moieties are employed. In some embodiments, each organic substituent moiety comprises between 1 and 4, or between 5 and 8 carbon atoms. Suitable organic and inorganic substituent moieties include, but are not limited to, hydroxyl, halogens, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, heteroaryl, substituted heteroaryl, aryl or substituted aryl. When more than one substituent group is present then they can be the same or different.

Abbreviations used herein include:

The term “alkoxy” as used herein denotes an alkyl moiety, defined above, attached directly to a oxygen to form an ether residue. Examples include methoxy, ethoxy, n-propoxy, iso-propoxy, n-butoxy, t-butoxy, iso-butoxy and the like.

The term “substituted alkoxy” denotes an alkoxy moiety of the above definition that is substituted with one or more groups, but preferably one or two substituent groups including hydroxyl, cycloalkyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy. When more than one group is present then they can be the same or different.

The term “mono-substituted amino” denotes an amino (—NH₂) group substituted with one group selected from alkyl, substituted alkyl or arylalkyl wherein the terms have the same definitions found throughout.

The term “di-substituted amino” denotes an amino substituted with two moieties that can be the same or different selected from aryl, substituted aryl, alkyl, substituted alkyl or arylalkyl, wherein the terms have the same definitions found throughout. Some examples include dimethylamino, methylethylamino, diethylamino and the like.

The term “haloalkyl” denotes a alkyl moiety, defined above, substituted with one or more halogens, preferably fluorine, such as a trifluoromethyl, pentafluoroethyl and the like.

The term “haloalkoxy” denotes a haloalkyl, as defined above, that is directly attached to an oxygen to form a halogenated ether residue, including trifluoromethoxy, pentafluoroethoxy and the like.

The term “acyl” denotes a moiety of the formula —C(O)—R that comprises a carbonyl (C═O) group, wherein the R moiety is an organic moiety having a carbon atom bonded to the carbonyl group. Acyl moieties contain 1 to 8 or 1 to 4 carbon atoms. Examples of acyl moieties include but are not limited to formyl, acetyl, propionyl, butanoyl, iso-butanoyl, pentanoyl, hexanoyl, heptanoyl, benzoyl and like moieties.

The term “acyloxy” denotes a moiety containing 1 to 8 carbons of an acyl group defined above directly attached to an oxygen such as acetyloxy, propionyloxy, butanoyloxy, iso-butanoyloxy, benzoyloxy and the like.

The term “aryl” denotes an unsaturated and conjugated aromatic ring moiety containing 6 to 18 ring carbons, or preferably 6 to 12 ring carbons. Many aryl moieties have at least one six-membered aromatic “benzene” moiety therein. Examples of such aryl moieties include phenyl and naphthyl.

The term “substituted aryl” denotes an aryl ring moiety as defined above that is substituted with or fused to one or more organic or inorganic substituent moieties, which include but are not limited to a halogen, alkyl, substituted alkyl, haloalky, hydroxyl, cycloalkyl, substituted cycloalkyl, cycloalkenyl, substituted cycloalkenyl, amino, mono-substituted amino, di-substituted amino, acyloxy, nitro, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy or haloalkoxy, aryl, substituted aryl, heteroaryl, heterocyclic ring, substituted heterocyclic ring moiety, wherein the terms are defined herein. Substituted aryl moieties can have one, two, three, four, five, or more substituent moieties. The substituent moieties can be not be of unlimited size or molecular weight, and each organic moiety can comprise 15 or fewer, 10 or fewer, or 4 or fewer carbon atoms unless otherwise expressly contemplated by the claims.

The term “heteroaryl” denotes an aryl ring moiety as defined above, wherein at least one of the carbons of the aromatic ring has been replaced with a heteroatom, which include but are not limited to nitrogen, oxygen, and sulfur atoms. Heteroaryl moieties include 6 membered aromatic ring moieties, and can also comprise 5 or 7 membered aromatic rings, or bicyclic or polycyclic heteroaromatic rings as well. Examples of heteroaryl moieties include pyridyl, bipyridyl, furanyl, and thiofuranyl residues. It is to be understood that the heteroaryl moieties can optionally be substituted with one or more organic or inorganic substituent moieties bound to the carbon atoms of the heteroaromatic rings, as described hereinabove for substituted aryl moieties. Substituted heteroaryl moieties can have one, two, three, four, five, or more substituent organic or inorganic moieties, in a manner analogous to the substituted aryl moieties defined herein. The substituent moieties cannot be of unlimited size or molecular weight, and each organic substituent moiety can comprise 15 or fewer, 10 or fewer, or four or fewer carbon atoms unless otherwise expressly contemplated by the claims.

The term “halo,” “halide,” or “halogen” refers to a fluoro, chloro, bromo or iodo atom or ion.

The term “heterocycle” or “heterocyclic”, as used in the specification and concluding claims, refers to a moiety having a closed ring structure comprising 3 to 10 ring atoms, in which at least one of the atoms in the ring is an element other than carbon, such as, for example, nitrogen, sulfur, oxygen, silicon, phosphorus, or the like. Heterocyclic compounds having rings with 5, 6, or 7 members are common, and the ring can be saturated, or partially or completely unsaturated. The heterocyclic compound can be monocyclic, bicyclic, or polycyclic. Examples of heterocyclic compounds include but are not limited to pyridine, piperidine, thiophene, furan, tetrahydrofuran, and the like. The term “substituted heterocyclic” refers to a heterocyclic moiety as defined above having one or more organic or inorganic substituent moieties bonded to one of the ring atoms.

The term “carboxy”, as used in the specification and concluding claims, refers to the —C(O)OH moiety that is characteristic of carboxylic acids. The hydrogen of the carboxy moieties is often acidic and (depending on the pH) often partially or completely dissociates, to form an acid H+ ion and a carboxylate anion (—CO₂—), wherein the carboxylate anion is also sometimes referred to as a “carboxy” moiety.

It is understood that when a chiral atom is present in a compound disclosed herein, both separated enantiomers, racemic mixtures and mixtures of enantiomeric excess are within the scope of the present disclosure. As defined herein, racemic mixture is an equal ratio of each of the enantiomers, whereas an enantiomeric excess is when the percent of one enantiomer is greater than the other enantiomer, all percentages are within the scope of the present disclosure. Furthermore, when more than one chiral atom is present in a compound then the enantiomers, racemic mixtures, mixtures of enantiomeric excess and diastereomeric mixtures are within the scope of the present disclosure.

### Compounds

The compounds described below are salts, and can be used for the treatment of various diseases as disclosed elsewhere herein. As will be appreciated by those of ordinary skill in the art, the salts comprise a mixture of cations and anions whose total number of positive and negative charges are electrically balanced. More particularly however the salts disclosed herein have one or more molecules or cations having the Formula (I) illustrated below
a) at least one molecule having the formula:

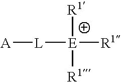

wherein:
i) A is at least one group capable of functioning as an anti-Signaling or anti-oxidant or reduced anti-oxidant, comprising a hydroquinone, dihydroquinone, quinone, quinol, phenol, diamine, triterpene, tetracycline, chromanol, chromanone, chroman tempol, tempol-H or a pro-drug thereof, having from 2 to 30 carbon atoms;
ii) L or L* is a linking group comprising from 0 to 50 carbon atoms which may not or may have a pH-sensitive *carbodiamide linker;
iii) E is no atom or a nitrogen or phosphorous;
iv) R^{1′}, R^{1″}, and R^{1′″} are each independently chosen from organic radicals comprising from 0 to 12 carbon atoms; and
b) at least one anion having the formula X^{⊖} wherein the cation and the anion, if present, are present in an amount sufficient to form a neutral, pharmaceutically acceptable salt.

The various genera, subgenera, and species of the compounds of Formula (I) share at least the features disclosed above, and have related functions and utilities, but can differ in specific structural features, as described below.

### The “Anti-Signaling, Anti-Oxidative Stress Modulating or Anti-oxidant”=“A” Moieties

In some embodiments, the compounds of the present disclosure comprise at least one antioxidant moiety “A” which comprises at least one or more hydroquinones, quinones, modified quinines, plastoquinones, quinols, chromanols, chromanones, chromans, phenols, diamines, triterpenes, tempols, tempol-H or carbothioamides bonded therein or thereto.

Hydroquinones and relevant quinones have the chemical structures shown below:

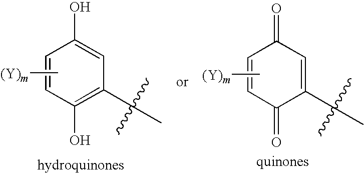

while an example of a phenol is the chroman 6-hydroxy-2,5,7,8-tetramethyl-chroman-2-yl having the formula:

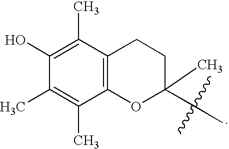

Accordingly, the “A” moieties of the cationic salts described herein, which comprise one or more quinone moieties which can reduce superoxide radical anions in the cell, to form hydrogen peroxide which can be dealt with by anti-oxidant defense enzymes in the cell, and therefore serve to function as “Anti-oxidants.” The quinone and other moieties are part of a larger A moiety, which in many embodiments can comprise between 4 and 30 carbon atoms, or, 6 to 24 carbon atoms, or 7 to 18 carbon atoms, or from 8 to 12 carbon atoms.

In some embodiments, the A moieties have the formula:

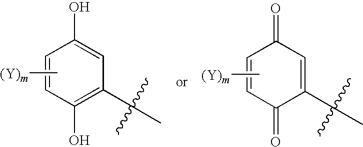

wherein Y is optionally present, and can be one or more electron activating moieties chosen from:
i) C₁-C₄ linear, branched, or cyclic alkyl;
ii) C₁-C₄ linear, branched, or cyclic haloalkyl;
iii) C₁-C₄ linear, branched, or cyclic alkoxy;
iv) C₁-C₄ linear, branched, or cyclic haloalkoxy; or
v) —N(R²)₂, each R² is independently hydrogen or C₁-C₄ linear or branched alkyl.

The index m indicates the number of Y units present and the value of m is from 0 to 3.

In one embodiment Y is an electron activating moiety independently chosen from methyl, ethyl, n-propyl, iso-propyl, n-butyl, sec-butyl, iso-butyl, tent-butyl, methoxy, ethoxy, n-propoxy, iso-propoxy, n-butoxy, and, tert-butoxy.

In one embodiment Y is chosen from 1 to 3 methyl and/or methoxy units. An example includes the following hydroquinone and quinone radicals having the formula:

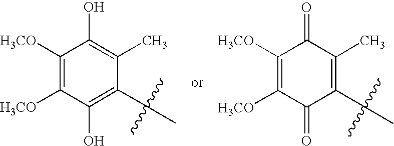

### The Ammonium or Phosphonium Cationic Moieties

The compounds useful for the methods of the disclosure comprise none or one or more cationic or poly cationic moieties. The cationic moieties carry a positive charge, which, while not being bound by theory, is believed to cause the desirable selective accumulation of the resultant compounds in the mitochondria, because of the large mitochondrial membrane potential of 150-170 mV, and the resulting electrostatic attractions. Again, while not being bound by theory, it has been found that the selective accumulation of the cationic salts disclosed herein is also improved if the cationic moieties comprise relatively large and/or lipophilic organic substituent moieties, so that the resulting cationic group is relatively lipophilic when considered as a whole, even if the A group is not lipophilic. One of ordinary skill in the art will recognize that many relatively lipophilic cationic groups can be synthesized, especially from compounds comprising nitrogen or phosphorus atoms, and it is evident that many such cationic moieties could be linked in various ways to the anti-oxidant or reduced antioxidant A moieties, and provide a cation that might be useful in the practice of the methods described herein. More particularly however, in many embodiments of the salts and/or cationic compounds of Formula (I) have quaternary ammonium or phosphonium moieties, having the formula:

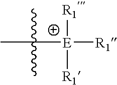

wherein:
E is a nitrogen or phosphorus atom; and R₁′, R₁″, and R₁′″ are each independently organic moieties comprising from 1 to 12 carbon atoms.

In many embodiments, the compounds of Formula (I) can have R₁′, R₁″, and R₁′″ are each independently selected from alkyl, aryl, heteroaryl, or aralkyl moieties, which may be unsubstituted, or optionally substituted with one or two independently selected substituent moieties, which include but are not limited to hydroxyl, halogen, amino, amino, dimethylamino, alkyl, hydroxyalkyl, alkoxy, alkoxylalkyl, carboxy, or carboxyalkyl moieties. Non-limiting examples of the optional substituents for R₁′, R₁″, and R₁′″ include:
i) C₁-C₄ linear branched alkyl; for example, methyl (C₁), ethyl (C₂), n-propyl (C₃), iso-propyl (C₃), n-butyl (C₄), sec-butyl (C₄), iso-butyl (C₄), and tent-butyl (C₄);
ii) C₁-C₄ linear or branched alkoxy; for example, methoxy (C₁), ethoxy (C₂), n-propoxy (C₃), iso-propoxy (C₃), n-butoxy (C₄), sec-butoxy (C₄), iso-butoxy (C₄), and tert-butoxy (C₄);
iii) halogen; for example, —F, —Cl, —Br, —I, and mixtures thereof;
iv) amino and substituted amino; for example, —NH₂, —NH₂, —NHCH₃, —NHCH₃, and —N(CH₃)₂;
v) hydroxyl; —OH;
vi) C₁-C₄ linear or branched hydroxyalkyl; for example, —CH₂OH, —CH₂CH₂OH, —CH₂CH₂CH₂OH, and —CH₂CHOHCH₃;
vii) C₁-C₄ linear or branched alkoxyalkyl; for example, —CH₂OCH₃, —CH₂CH₂OCH₃, —CH₂CH₂CH₂OCH₃, and —CH₂CH(OCH₃)CH₃;
viii) carboxy or carboxylate, for example, —CO₂H or the anionic equivalent carboxylate moieties —CO₂⁻; and
xi) carboxyalkyl, for example, —CH₂CO₂H, —CH₂CH₂CO₂H, —CH₂CO₂CH₃, —CH₂CH₂CO₂CH₃, and —CH₂CH₂CH₂CO₂CH₃.

In related embodiments, R₁′, R₁″, and R₁′″ are each independently selected from alkyl, aryl, or benzyl moieties optionally substituted with one or two independently selected hydroxyl, halogen, amino, diamino, dimethylamino, diethylamino, alkyl, hydroxyalkyl, alkoxy, alkoxylalkyl, carboxy, or carboxyalkyl moieties.

In other related embodiments, R₁′, R₁″, and R₁′″ are independently selected from C₄-C₁₀ alkyl or phenyl moieties, which can optionally be substituted with one or two independently selected substituent moieties, which can include but are not limited to hydroxyl, halogen, amino, diamino, dimethylamino, diethylamino, alkyl, hydroxyalkyl, alkoxy, alkoxylalkyl, cyano, carboxy, or carboxyalkyl moieties. In additional embodiments, R₁′, R₁″, and R₁′″ can be independently selected from C₄-C₁₀ alkyl or phenyl moieties. In some additional embodiments R₁′, R₁″, and R₁′″ are independently selected from C₄-C₁₀ alkyl. In yet other related embodiments R₁′, R₁″, and R₁′″ are each n-C₄H₉ moieties.

In some embodiments of the compounds of Formula (I) having phosphonium cations, R₁′, R₁″, and R₁′″ are each phenyl moieties, to produce triphenyl phosphonium cations having the formula:

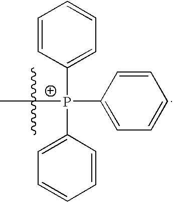

In alternative but related embodiments, R₁′, R₁″, and R₁′″ are each benzyl moieties, to produce tribenzyl phosphonium cations having the formula:

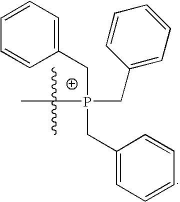

Other embodiments of the cations of Formula (I) relates to quaternary ammonium cations i.e. wherein E is a nitrogen atom. In some such embodiments, R₁′, R₁″, and R₁′″ are each independently selected from alkyl, aryl, heteroaryl, or aralkyl moieties, which can be optionally substituted with one or two independently selected substituent moieties, which include but are not limited to hydroxyl, halogen, amino, dimethylamino, diethylamino, alkyl, hydroxyalkyl, alkoxy, alkoxylalkyl, cyano, carboxy, or carboxyalkyl moieties. Non-limiting examples of the R₁′, R₁″, and R₁′″ substituents include:
i) C₁-C₄ linear branched alkyl; for example, methyl (C₁), ethyl (C₂), n-propyl (C₃), iso-propyl (C₃), n-butyl (C₄), sec-butyl (C₄), iso-butyl (C₄), and tent-butyl (C₄);
ii) C₁-C₄ linear or branched alkoxy; for example, methoxy (C₁), ethoxy (C₂), n-propoxy (C₃), iso-propoxy (C₃), n-butoxy (C₄), sec-butoxy (C₄), iso-butoxy (C₄), and tert-butoxy (C₄);
iii) halogen; for example, —F, —Cl, —Br, —I, and mixtures thereof;
iv) amino and substituted amino; for example, —NH₂, —NH₂, —NHCH₃, —NHCH₃, and —N(CH₃)₂;
v) hydroxyl; —OH;
vi) C₁-C₄ linear or branched hydroxyalkyl; for example, —CH₂OH, —CH₂CH₂OH, —CH₂CH₂CH₂OH, and —CH₂CHOHCH₃;
vii) C₁-C₄ linear or branched alkoxyalkyl; for example, —CH₂OCH₃, —CH₂CH₂OCH₃, —CH₂CH₂CH₂OCH₃, and —CH₂CH(OCH₃)CH₃; viii) carboxy; or carboxylate, for example, —CO₂H or the anionic equivalent carboxylate moieties —CO₂⁻; and
xi) carboxyalkyl, for example, —CH₂CO₂H, —CH₂CH₂CO₂H, —CH₂CO₂CH₃, —CH₂CH₂CO₂CH₃, and —CH₂CH₂CH₂CO₂CH₃.

In additional embodiments of the cations of Formula (I), wherein E is nitrogen, R₁′, R₁″, and R₁′″ are each independently selected from alkyl aryl, or benzyl moieties, which can be optionally substituted with one or two independently chosen substituent moieties, which include but are not limited to hydroxyl, halogen, amino, dimethylamino, diethylamino, alkyl, hydroxyalkyl, alkoxy, alkoxylalkyl, carboxy, or carboxyalkyl moieties.

In another embodiment R₁′, R₁″, and R₁′″ are independently selected from C₄-C₁₀ alkyl or phenyl moieties optionally substituted with one or two independently selected hydroxyl, halogen, amino, dimethylamino, alkyl, hydroxyalkyl, alkoxy, alkoxylalkyl, carboxy, or carboxyalkyl moieties. In one further aspect of this embodiment R₁′, R₁″, and R₁′″ are independently selected from C₄-C₁₀ alkyl or phenyl moieties; and in one further embodiment R₁′, R₁″, and R₁′″ are independently selected from C₄-C₁₀ alkyl.

In yet another embodiment of cations wherein E is nitrogen, R₁′, R₁″, and R₁′″ are each n-C₄H₉ moieties.

### The “L” or “*L” Linker Moiety

The cations of Formula (I) comprise a linker moiety “L”, which connects the “A” moiety and the cationic moiety. The exact structure and size of the L moieties can vary considerably, and many variations of the L moieties are within the scope of the embodiments disclosed herein. In some the L moieties are often organic moieties, and can comprise a wide variety of structures. In many embodiments it is desirable that the L moiety be of sufficient size and character that it provides some space and/or flexibility in the connection between the A and cation groups, but does not become of such high molecular weight so as to impair the water solubility or trans-membrane absorbability of the resulting cations.

Accordingly, in some embodiments, the L moiety, when considered as a whole, comprises from 4 to 50 carbon atoms, or from 4 to 30 carbon atoms, or from 4 to 20 carbon atoms. In some embodiments, the L moiety comprises from 0 to 18 carbon atoms, or from 8 to 12 carbon atoms.

In one embodiment L has the formula:

—[C(R^{2a})(R^{2b})]ⱼ[W]ₖ[C(R^{3a})(R^{3b})]ₙ[Z]ₚ[C(R^{4a})(R^{4b})]_{q}—

R^{2a}, R^{2b}, R^{3a}, R^{3b}, R^{4a}, and R^{4b} are each independently chosen from:
i) hydrogen;
ii) substituted or unsubstituted C₁-C₁₂ linear, branched, or cyclic alkyl;
iii) substituted or unsubstituted C₁-C₁₂ linear, branched, or cyclic alkenyl;
iv) substituted or unsubstituted C₁-C₁₂ linear or branched alkynyl;
v) —C(O)OR⁵;
vi) —C(O)R⁶;
vii) —OR⁷;
viii) —N(R^{8a})(R^{8b});
ix) —C(O)N(R^{9a})(R^{9b});
x) —CN;
xi) —NO₂;
xii) —SO₂R¹⁰;
R⁵, R⁶, R⁷, R⁸, R⁹, and R¹⁰ are each independently chosen from:
   a) hydrogen;
   b) substituted or unsubstituted C₁-C₁₂ linear, branched, or cyclic alkyl;
   c) substituted or unsubstituted C₆ or C₁₀ aryl;
W and Z are each independently chosen from:
i) -M-;
ii) —C(=M)-;
iii) —C(=M)M-;
iv) -MC(=M)-;
v) -MC(=M)M-;
vi) -MC(=M)C(=M)M-; or
vii) -MC(=M)MC(=M)M-;
   wherein each M is independently chosen form O, S, and NR¹¹; R¹¹ is hydrogen, hydroxyl, or C₁-C₄ linear or branched alkyl; the indices j, n, and q are each independently from 0 to 30, provided j+n+q is equal to from 4 to 30; the indices k and p are independently 0 or 1; and L can comprise one or more units having the formula:

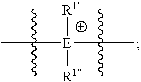

E, R^{1′}, and R^{1″} are the same as defined herein above.

In one embodiment of linking units the sum of the indices j, n, and q are from 4 to 24. In a further embodiment of linking units the sum of the indices j, n, and q are from 5 to 20. In a further embodiment of linking units the sum of the indices j, n, and q are from 6 to 16. In a further embodiment of linking units the sum of the indices j, n, and q are from 7 to 16. In a further embodiment of linking units the sum of the indices j, n, and q are from 8 to 12. In a further embodiment of linking units the sum of the indices j, n, and q is equal to 10.

In one embodiment, L has the formula:

—[C(R^{3a})(R^{3b})]ₙ—

R^{3a} and R^{3b} are each independently chosen from:
i) —H;
ii) C₁-C₄ linear or branched alkyl;
   the index n is from 4 to 30.

This embodiment of L units provides for the following compounds:

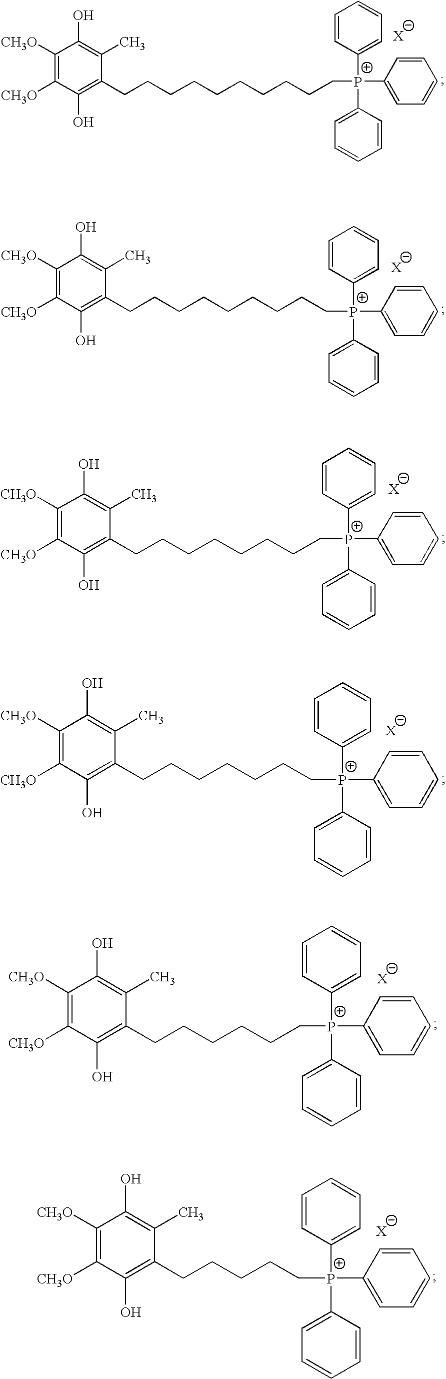

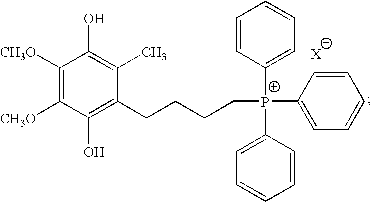

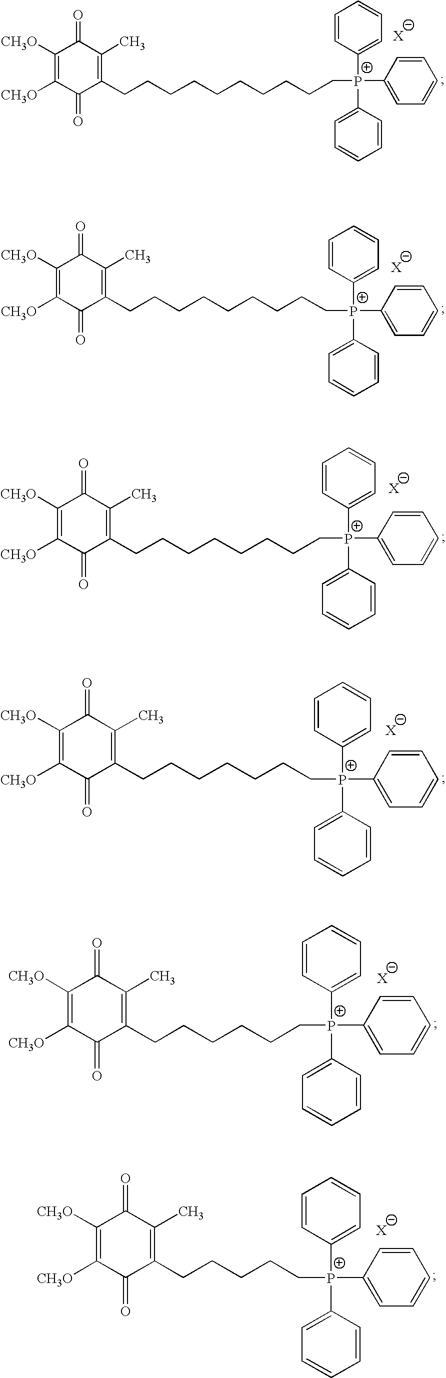

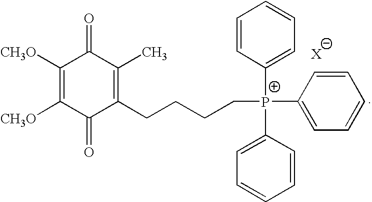

In some embodiments, the L moieties comprise only methylene or polymethylene moieties, i.e., —(CH₂)ₙ— moieties. Some embodiments provide L having from 4 to 24 carbon chain atoms, for example, —(CH₂)ₙ—, wherein the index n is from 4 to 24. Other embodiments relates to L having from 5 to 20 carbon atoms, from 6 to 16 carbons atoms, from 7 to 16 carbon atoms, and from 8 to 12 carbon atoms. One particular embodiment relates to L units having 10 carbon atoms (n=10), for example, 10 methylene units having the formula:

—CH₂CH₂CH₂CH₂CH₂CH₂CH₂CH₂CH₂CH₂—.

In another embodiment L has the formula:

—[C(R^{2a})(R^{2b})]ⱼ[C(R^{3a})(R^{3b})]ₙ[C(R^{4a})(R^{4b})]_{q}—

one non-limiting example of which has the formula:

—[CH₂]₂[C(R^{3a})(R^{3b})][CH₂]_{q}—

thereby providing compounds having the formula:

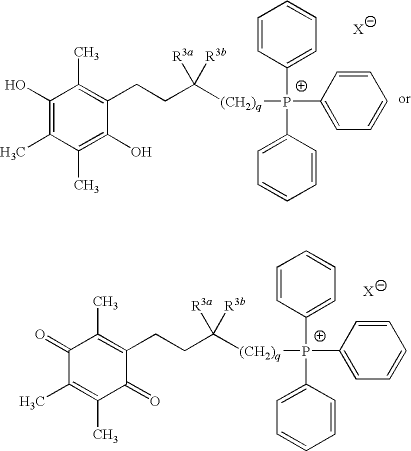

wherein q is from 1 to 20 and R^{3a} and R^{3b} are each independently chosen from hydrogen, methyl, ethyl, propyl and hydroxyl.

Non-limiting examples have the formula:

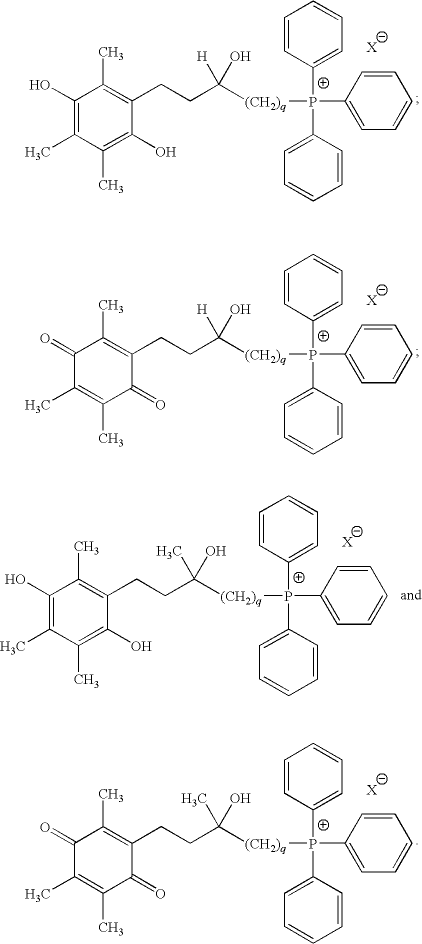

Non-limiting examples include compounds having the formula:

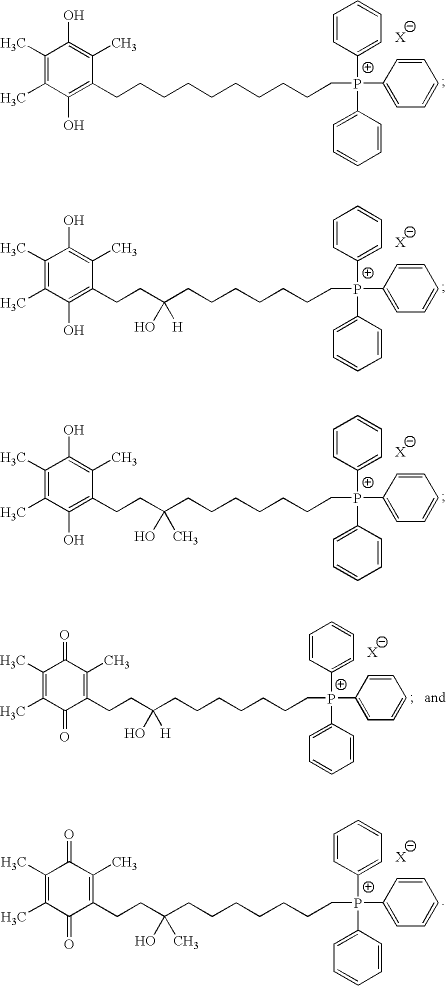

Nevertheless, the L moieties can further comprise in the carbon chain from 1 to 10 additional atoms or groups independently selected from O, S, S(O)—, —S(O)₂—, —NH—, —NCH₃—, —C(O)—, or —C(O)O—. For example, in some embodiments, L can be a polyalkylene moiety, or a polyethylene glycol moiety, having the formula:

—(CH₂CH₂O)ₙCH₂CH₂—

wherein n is an integer from 0 to 3.

### The Xⁿ⁻ Anions

The salt compounds comprising the cations of Formula (I) also comprise an anion Xⁿ⁻, wherein n is an integer from 1 to 4, corresponding to mono-anions, di-anions, tri-anions, and tetra-anions. The first embodiment of X⁻ relates to inorganic anion moieties. Mono-anionic inorganic anions include any halide anion, such as fluoride, chloride, bromide, or iodide; nitrate, hydrogen sulfate; dihydrogen phosphate, and the like. Dianionic inorganic cations can include carbonate, sulfate or hydrogen phosphate, and tri-anionic inorganic anions include phosphates.

In other embodiments of the Xⁿ⁻ anions, the anions are organic anions. Non-limiting examples of organic anion moieties that can be employed to form the salts from the cations of Formula (I) include organosulphates such as methylsulphonate (mesylate), trifluoromethylsulfonate (triflate), benzenesulphonate, toluenesulphonate (tosylate), or purely organic anions, often formed by the neutralization of organic acids, such as fumarate, maleate, maltolate, succinate, acetate, benzoate, oxalate, citrate, or tartrate anions.

Those of ordinary skill in the art will recognize that both the cations of Formula (I) and the corresponding Xₙ⁻ anions must be combined in appropriate ratios so as to produce isolated and electrically neutral salt compounds that can be isolated and used in the methods and compositions disclosed herein. Accordingly, one way of expressing the condition of electrical neutrality when applied to the salt compounds as a whole is to recognize that such salt compounds can have the formula:

N[cation]^{m+}M[anion]ⁿ⁺

wherein the indices M, N, m and n are each independently from 1 to 4, provided that the product (M×n)=(m×N) thereby forming a neutral salt.

The present disclosure further relates to compounds comprising:
a) a cation having the formula:

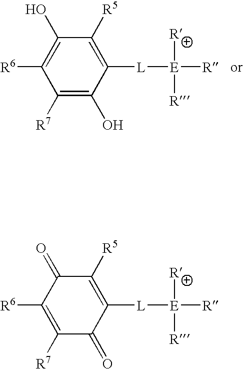

wherein
i) L is a linking group comprising from 4 to 30 carbon atoms as defined herein;
ii) E is nitrogen or phosphorous;
iii) R′, R″, and R′″ are each independently chosen from organic radicals comprising from 1 to 12 carbon atoms as defined herein;
iv) R⁵, R⁶, and R⁷ are each independently hydrogen or an electron activating moiety as defined herein; and
b) at least one anion having the formula X^{⊖} as further defined herein, and wherein the cation and the anion are present in an amount sufficient to form a neutral, pharmaceutically acceptable salt.

One embodiment of the present disclosure relates to compounds wherein R⁵, R⁶, and R⁷ are each independently hydrogen or an electron activating moiety independently chosen from:
i) C₁-C₄ linear, branched, or cyclic alkyl;
ii) C₁-C₄ linear, branched, or cyclic haloalkyl;
iii) C₁-C₄ linear, branched, or cyclic alkoxy;
iv) C₁-C₄ linear, branched, or cyclic haloalkoxy; or
v) —N(R²)₂, each R² is independently hydrogen or C₁-C₄ linear or branched alkyl.

One embodiment relates to compounds wherein each electron activating moiety is independently chosen from methyl, ethyl, n-propyl, iso-propyl, n-butyl, sec-butyl, iso-butyl, tert-butyl, methoxy, ethoxy, n-propoxy, iso-propoxy, n-butoxy, and, tert-butoxy.

Particular generic examples of this embodiment include:

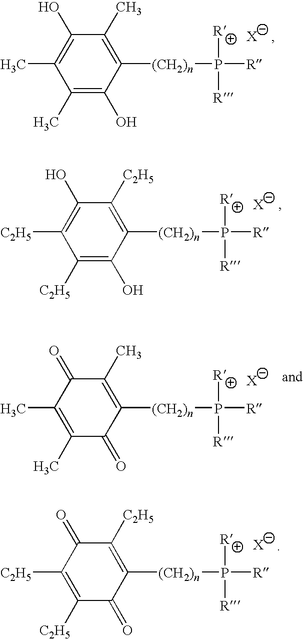

Examples of specific compounds according to this embodiment include:

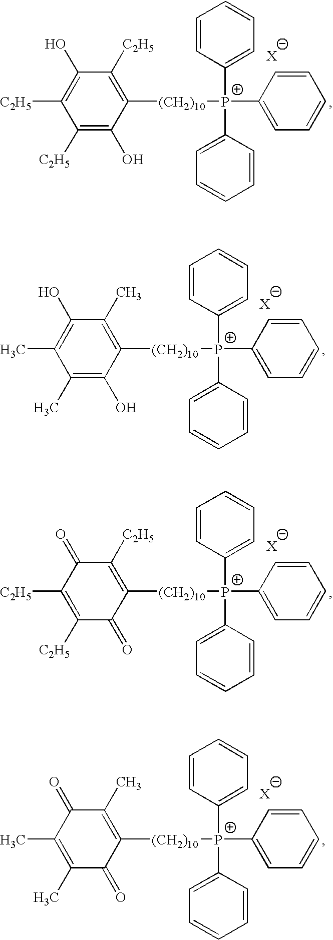

Another embodiment includes compounds having the formula:

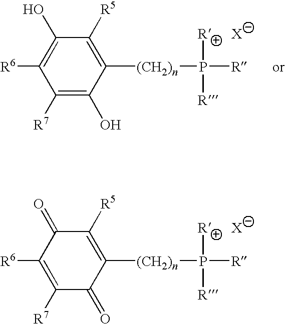

wherein the index n is from 4 to about 24, or the index n is from 5 to 20, or the index n is from 6 to 16, or the index n is from 7 to 16 or the index n is from 8 to 12. One example of this embodiment encompasses compounds wherein the index n is equal to 10.

One embodiment relates to R^{1′}, R^{1″}, and R^{1′″} units that are each independently chosen from:
i) C₆ or C₁₀ substituted or unsubstituted aryl; or
ii) C₇-C₁₂ substituted or unsubstituted arylalkylene;
each of which is optionally substituted with one or more units independently chosen from:
i) methyl, ethyl, n-propyl, iso-propyl, n-butyl, or tent-butyl;
ii) methoxy, ethoxy, n-propoxy, iso-propoxy, n-butoxy, or tert-butoxy;
iii) fluoro, chloro, bromo, iodo;
iv) —NH₂, —NHCH₃, —N(CH₃)₂, —NH(CH₂CH₃), —N(CH₂CH₃)₂;
v) —C(O)OH, —CO₂CH₃, —CO₂CH₂CH₃, —CO₂CH₂CH₂CH₃;
vi) —COCH₃, —COCH₂CH₃, —COCH₂CH₂CH₃;
vii) —C(O)NH₂, —C(O)NHCH₃, —C(O)N(CH₃)₂, —C(O)NH(CH₂CH₃), —C(O)N(CH₂CH₃)₂;
viii) —CN;
ix) —NO₂; and
xii) —SO₂OH, —SO₂CH₃; —SO₂NH₂.

Examples of this embodiment includes R′, R″, and R′″ units that are each independently chosen from substituted phenyl or benzyl. Non-limiting examples of this embodiment include R′, R″, and R′″ units that are each phenyl or benzyl.

### Synthesis of the Compounds Disclosed Herein

Various methods and/or strategies have been disclosed in the literature and can be employed in the synthesis or production of salts having cations of Formula (I) and Xⁿ⁻ anions, as described above. Several such synthetic methods and/or strategies will be disclosed herein below.

Scheme I outlines a process of preparing the compounds of the present disclosure.

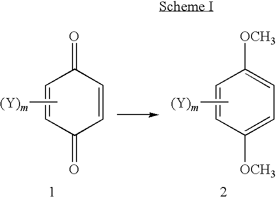

Reagents and conditions: (a)(i) NaBH₄, MeOH; (ii) (CH₃)₂SO₄, NaOH.

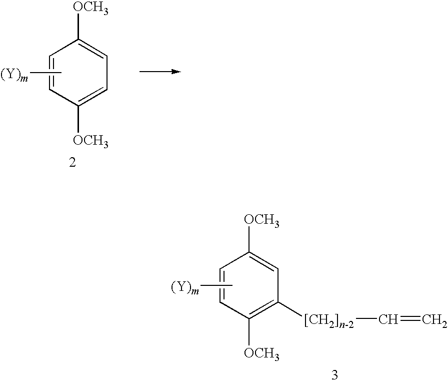

Reagents and conditions: (b)(i) n-BuLi, TMEDA; (II) CuCN, CH₂═CH(CH₂)ₙ₋₂Br.

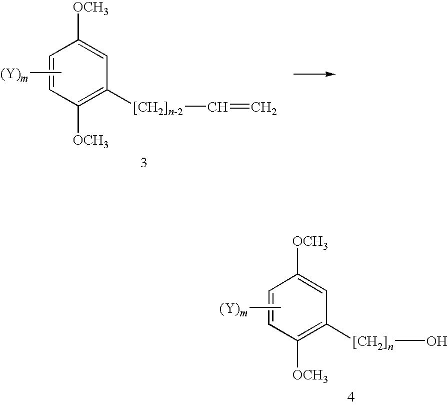

Reagents and conditions: (c) 9-BBN

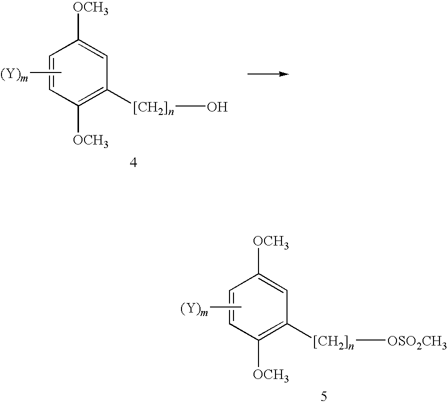

Reagents and conditions: (d) CH₃SO₄Cl.

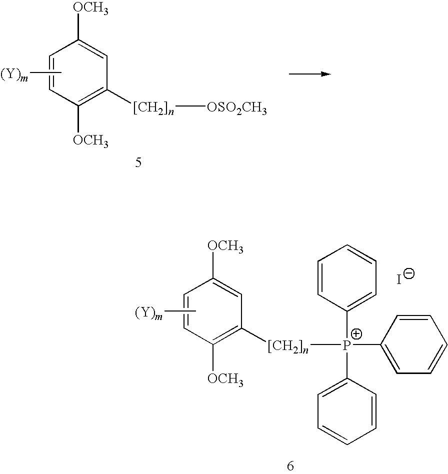

Reagents and conditions: (e)(i) NaI; (ii) P(C₆H₅)₃.

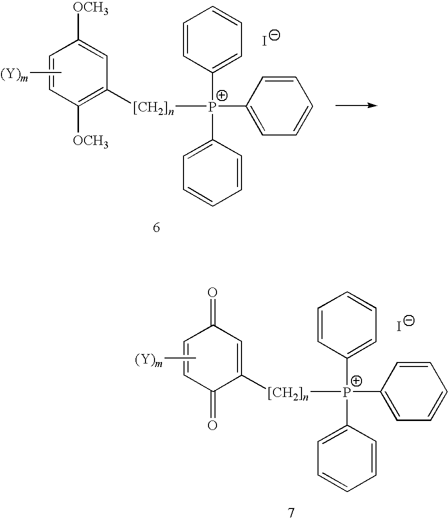

Reagents and conditions: (f) Ce(NH₄)₂(NO₃)₆.

### Example 1

The following is a general procedure for preparing analogs of the present disclosure wherein the index n is from 4 to 20 and the linking group comprises methylene units.

Starting materials 1, for example, 2,3-methoxy-5-methyl-1,4-benzoquionone can be prepared according to the procedure of Lipshutz, B. H. et al., (1998) Tetrahedron 54, 1241-1253, incorporated herein by reference to the extent it is relevant.

Intermediate 2 is prepared by reaction of starting material 1, for example reduction of 2,3-dimethoxy-5-methyl-1,4-benzoquinone to 2,3,4,5-tetrahydroxytoluene by the procedure of Carpino, L. A. et al., (1989) J. Org. Chem. 54, 3303-3310, incorporated herein by reference in its entirety, using sodium borohydride in methanol, followed by methylation with NaOH/(CH₃)₂SO₄ according to the procedure of Lipshutz.

Preparation of Intermediate 3: A solution of Intermediate, 2, (30 mmol) in dry hexane (80 mL) and N,N,N′,N′-tetramethylethylenediamine (8.6 mL) is placed in a dry Schlenk tube under inert atmosphere. A hexane solution of n-butyl lithium (1.6 M, 26.2 mL) is slowly added at room temperature and the mixture is then cooled and stirred at 0° C. for about one hour. The solution is then cooled to −78° C. and dry tetrahydrofuran (250 mL) is added. At this point the formulator can analyze the reaction solution to determine if the ring is fully metalated before proceeding. The contents of the reaction vessel is then transferred to a second Schlenk tube containing CuCN (6 mmol) under inert atmosphere. The mixture is then warmed to 0° C. for 10 minutes, then re-cooled to −78° C. The w-bromoolefin (25% to 50% excess depending upon the reactivity of the ω-bromoolefin) is added. The reagent will vary depending upon the length of the linking group, —[CH₂]ₙ—. For the final compound, wherein the index n is equal to 10,10-bromodec-1-ene is used for this step. Once the ω-bromoolefin is added the solution is allowed to warm and stir at room temperature until the formulator determines the reaction is complete. The reaction is then quenched with 10% aqueous NH₄Cl (˜75 mL), and the resulting solution extracted with solvent several times. The combined solvent extracts are combined and washed with water, 10% aqueous NH₄OH, and brine. The organic phase can be dried over any suitable drying agent after which the solvent is removed under reduced pressure. At this point the formulator can purify the crude product or proceed if it is determined the material has sufficient purity.

Intermediate 4: A solution of Intermediate 3 (33 mmol) in dry THF (45 mL) is added dropwise over 20 minutes to a stirred suspension of 9-borabicyclo[3.3.1]nonane (9-BBN) in THF (40 mmol) at 25° C. The resulting solution is stirred at room temperature then heated if necessary from about 60° C. to about 65° C. until the formulator determines the reaction is complete. The mixture is cooled to 0° C. and 3 M NOH (˜53 mL) is added dropwise. After addition is complete a 30% aqueous H₂O₂ solution (˜53 mL) is added. After allowing the solution to stir approximately 30 minutes at room temperature, the water phase is saturated with NaCl and extracted several times with THF. The organic phases are combined, washed with brine, and dried. The solvent in removed by evaporation to afford crude Intermediate 4. At this point the formulator can purify the crude product or proceed if it is determined the material has sufficient purity.

Preparation of Intermediate 5: A solution of Intermediate 4 (15 mmol) and triethylamine (30 mmol) in methylene chloride (50 mL) is stirred at room temperature then methanesulfonyl chloride (15.75 mmol) in methylene chloride (50 mL) is added dropwise over approximately 30 minutes, after which the reaction is allowed to stir until judged to be complete. The reaction solution is diluted with methylene chloride (50 mL) and the organic layer washed several times with water, then 10% aqueous NaHCO₃. The solution is then dried and concentrated in vacuo to afford the crude product. At this point the formulator can purify the crude product or proceed if it is determined the material has sufficient purity, however, the crude material can typically be used directly.

Preparation of Intermediate 6: The crude intermediate 5 (9.0 mmol) is mixed with a triphenylphosphine (15.6 mmol) and NaI (51.0 mmol) in a Kimax tube and sealed under argon. The mixture is then held at 70-74° C. with magnetic stirring for about 3 hours during which time there is a change in the mixture from a molten liquid to a glassy solid. The tube is then cooled and the residue treated with methylene chloride (30 mL). The suspension which typically results is filtered and the filtrate evaporated under reduced pressure. The resulting residue is dissolved in methylene chloride (minimal amount) and triturated with diethyl ether or pentane depending upon the choice of the formulator. The precipitate is filtered washed with the triturating solvent, and dried to afford the desired Intermediate 6.

Preparation of final analog: A solution of intermediate 6 (7.8 mmol) in methylene chloride (80 mL) is shaken with 10% aqueous NaNO₃ (50 mL) in a separatory funnel for about 5 minutes. The organic layer is separated, dried, filtered and concentrated in vacuo to afford the nitrate salt of Intermediate 6 (typically this conversion is 100%). The salt is dissolved in a mixture of acetonitrile and water (7:3, 38 mL) and stirred at 0° C. in an ice bath. Pyridine-2,6-dicarboxylic acid (39 mmol) is added followed by dropwise addition of a solution of ceric ammonium nitrate (39 mmol) in acetonitrile/water (1:1, 77 mL) over about 5 minutes. The reaction mixture is stirred in the cold for about 20 minutes than at room temperature for 10 minutes. The reaction mixture is then poured into water (200 mL) and extracted with methylene chloride (200 mL). The organic layer is dried, filtered, and concentrated to afford the final analog as the nitrate salt. The bromide salt is formed by dissolving the nitrate salt in methylene chloride (100 mL) and shaking it with a 20% aqueous KBr (50 mL). The organic layer is collected, dried, and concentrated to afford the final analog as the bromide salt.

### Example 2

### [10-(2,5-Dihydroxy-3,4-dimethoxy-6-methylphenyl)decyl]triphenylphosphonium bromide

2-(10-Hydroxydecyl)-5,6-dimethoxy-3-methylcyclohexa-2,5-diene-1,4-one (250 g, 740 mmol) is dissolved in methylene chloride (2.5 L) and the mixture is then cooled to 10° C. under an inert atmosphere. Triethylamine (125 g, 1.5 mol) is added in one portion and the mixture allowed to re-equilibrate to 10° C. A solution of methanesulfonyl chloride (94 g, 820 mmol) in methylene chloride (500 mL) is then added gradually at such a rate as to maintain an internal temperature of approximately 10-15° C. The reaction mixture is agitated for a further 15-20 minutes. The mixture is then washed with water (850 mL) and saturated with aqueous sodium bicarbonate solution (850 mL). The organic layer is evaporated to a red liquid under reduced pressure at 40-45° C. After drying for an additional 2-4 hours under high vacuum at ambient temperature, the crude product is used for the next step without further purification.

10-(4,5-Dimethoxy-2-methyl-3,6-dioxocyclohexa-1,4-dienyl)decyl methanesulfonate (310 g, 740 mmol) is dissolved in MeOH (2 L) and the mixture then cooled to 0-5° C. under an inert atmosphere. Sodium borohydride (30 gm, 790 mmol) is added portion-wise at such a rate as to ensure that the internal temperature does not exceed about 15° C. Completion of the reaction is accompanied by a color change of from red to yellow. The reaction mixture is agitated for a further 10-30 minutes and the reaction completeness is then checked. The mixture quenched with 2 L of 2M HCl and extracted three times with 1.2 L of methylene chloride. The combined organic phases are then washed once with water (1.2 L) and dried. The organic as is then evaporated to a yellow/brown syrup under reduced pressure at 40-45° C. The material is then dried at room temperature for an additional 2-8 hours to afford 304 g (98.9% yield) of the desired product which is used for the next step without further purification.

Triphenylphosphine (383 g, 1.46 mol) is added to 10-(4,5-dimethoxy-2-methyl-3,6-dioxocyclohexa-1,4-dienyl)decyl methanesulfonate (304 g, 730 mmol) in a round bottom flask. The flask is then attached to a rotary evaporator and the contents heated under vacuum in a bath with a temperature of 80-85° C. Once the mixture has formed a melt and degassing is no longer evident, the vacuum is displaced by an inert atmosphere and the mixture is spun gently in a bath set to 80-85° C. for approximately 3 days. The mixture is then cooled about room temperature and dissolved in methylene chloride (800 mL). Ethyl acetate (3.2 L) is then added in portions with gentle warming to precipitate the desired product away from an excess triphenylphosphine. The solvent volume is reduced and the remaining mixture is then cooled to room temperature and decanted. The remaining syrupy residue is then treated with ethyl acetates 3.2 L) twice more and then dried under high vacuum to afford 441 g (89.5% yield) of the desired product.

The crude material from above (440 g, 5.65 mol) is dissolved in methylene chloride (6 L) and the flask is purged with oxygen. The contents of the flask are vigorously stirred under the oxygen atmosphere for 30 minutes. A solution of 0.65 M NaNO₂ in dry dichloromethane (100 mL, 2 mol % NaNO₂) is added rapidly in one portion and the mixture is vigorously stirred under an oxygen atmosphere for 4-8 hours at room temperature. [If the reaction is deemed to be incomplete additional NaNO₂ can be added.] The solvent is removed by evaporation under reduced pressure to afford a red syrupy residue. This residue is dissolve in methylene chloride (2 L) at 40-45° C. Ethyl acetate (3.2 L) is then added in portions with gentle warming to precipitate the desired product. The oily residue is dried under high vacuum to afford 419 g (94% yield) of the desired product as a red glass.

### Biological Activity

The salts described above have been found to be potent compounds in a number of in vitro biological assays that correlate to or are representative of human diseases, especially diseases of uncontrolled cellular proliferation, including benign hyperplasia and various cancers.

The biological activity of the compounds described herein can be measured, screened, and/or optimized by testing the salts for their relative ability to kill or inhibit the growth of various human tumor cell lines and primary tumor cell cultures.

Tumor cell lines that can be employed for such tests include, but are not limited to, known cell lines that model cancers and/or diseases of uncontrolled cellular proliferation, such as:

For Leukemia: CCRF-CEM, HL-60 (TB), K-562, MOLT-4, RPMI-8226, and SR. Lung Cancer: A549/ATCC, EKVX, HOP-62, HOP-92, NCI-H226, NCI-H23, NCI-H322M, NCI-H460, and NCI-H522.

Colon Cancer: COLO 205, HCC-2998, HCT-116, HCT-15, HT-29, KM-12, and SW-620.

CNS Cancer: SF-268, SF-295, SF-539, SNB-19, SNB-75, U-231, U-235 and U-251.

Melanoma: LOX-IMVI, MALME-3M, M-14, SK-MEL-2, SK-MEL-28, SK-MEL-5, UACC-257, and UACC-62.

Ovarian Cancer: IGR-OVI, OVCAR-3, OVCAR-4, OVCAR-5, OVCAR-8, and SK-OV-3.

Renal Cancer: 786-0, A-498, ACHN, CAKI-1, RXF-393, RXF-631, SN12C, TK-10, and U0-31.

Prostate Cancer: DU-145, PC-3 CWR22 prostate Cancer: DU-145, PC-3 CWR22 Breast Cancer: MDA-MB-468, MCF 7, MCF7/ADR-RES, MDA-MB-231/ATCC, HS578T, MDA-MB-435, MDA-N, BT-549, and T-47D.

Pancreatic Cancer: PANC-1, Bx-PC3, AsPC-1.

After the compounds to be screened have been applied to one or more of the above cancer cell lines, the anti-cancer effectiveness in some embodiments is gauged using a variety of assay procedures known to those of ordinary skill in the art for measuring the number of live cells in the cultures as a function of time.

One well known procedure employs 3-(4,5-dimethylthiazol-2-yl)-2,5-diphenyltetrazolium bromide (“MTT”) to differentiate live cells from dead cells. The MTT assay is based on the production of a dark blue formazan product by active dehydrogenase in the mitochondria of live tumor cells. After exposure of cancer cells to the compounds to be screened for a fixed number of days, only living cells contain active dehydrogenases and produce dark blue formazan from MTT and are stained. The numbers of live cells is measured by absorbance of visible light by the formazan at 595 nm. Anti-cancer activity in some embodiments is reported as percent of the tumor cell growth in a culture treated with a placebo. These MTT assay procedures have an advantage over an in vivo assay with common laboratory animals such as mice, in that results are obtained within a week as opposed to requiring several weeks or months.

These MTT anti-cancer activity screening assay provides data regarding the general cytotoxicity of an individual compound. In particular, as described in the examples herein, active anti-cancer compounds can be identified by applying the compounds at a concentration of about 10 μM to one or more cultured human tumor cell lines, such as for example leukemia, lung cancer, colon cancer, CNS cancer, melanoma, ovarian cancer, renal cancer, prostate cancer, breast cancer, or pancreatic cancer, so as to kill or inhibit cell growth of the tumor cells.

In some embodiments of the present disclosure, the compounds described herein are considered to be biologically active for the treatment of a particular cancer if, when they are applied to a culture of one of the above cancer cell lines at a concentration of about 10 μM or less, for a period of at least about 5 days, the growth of the cancer cells is inhibited, or the cancer cells killed to the extent of about 50% or more, as compared to a control not comprising the compound of the present disclosure.

For DNA assay, each culture plate was thawed and equilibrated to room temperature under protection from light. Hoechst 33258 or Hoechst 33342 dye was then added to each well in 200 μL, of high salt TNE buffer (10 mM Tris, 1 mM EDTA, 2 M NaCl [pH 7.4]) at a final concentration of 6.7 μg/mL. After further incubation at room temperature for 2 hours under protection from light, culture plates were scanned on the CytoFluor 2350™ scanner using the 360/460 nm filter excitation and emission set. The DNA fluorescence intensity was used as a measure of cell growth.

In particular, the biological activity of two particular salts whose structures are shown below were assayed for their relevance to the treatment or inhibition of the growth of prostate cancers.

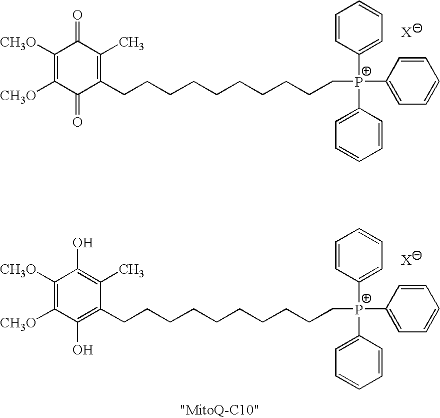

### Example 3

The effects of varying concentrations of Mito-Q drug on the growth of LNCaP and PC-3 cells over a period of 4 days was assayed using the Hoechst dye-DNA fluorescence assay described above. In these and all subsequent cell culture studies described below, each data point and its associated error bar are respectively, an average value and the standard deviation of data obtained from six wells of a 96-well plate run in duplicate in three separate sets of experiments.

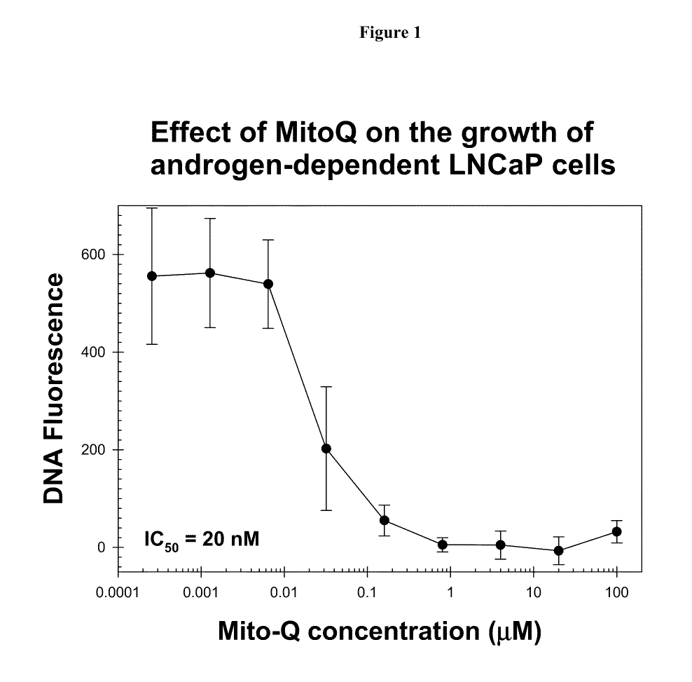

The results are shown in FIG. 1. Mito-Q-C10 treatment inhibits the growth of both LNCaP and PC-3 cells.

The inhibitory effect of Mito-Q-C10 on the oxidative stress level in LNCaP prostate tumor cells can also be determined by the ratio of DCF fluorescence/Hoechst dye-DNA fluorescence (Ripple M O, Henry W F, Rago R P, Wilding G. Prooxidant-antioxidant shift induced by androgen treatment of human prostate carcinoma cells. J Natl Cancer Inst. 1997 Jan. 1; 89(1):40-8). DCFH is oxidized to DCF by ROS to yield easily quantifiable ROS levels monitored by the green fluorescence of the DCF (6-carboxy-2′,7′-dichlorofluorescin diacetate) dye.

The DCF fluorescence in LNCaP cells treated with 1 nM of the androgen analog metribolone was normalized with the blue fluorescence of the Hoechst dye-DNA complex in the same cells at varying concentrations of Mito-Q-C10, in order to evaluate the level the oxidative stress per individual cell.

The inhibitory effect of Mito-Q-C10 on the oxidative stress level in LNCaP prostate tumor cells can also be determined by the ratio of DCF fluorescence/Hoechst dye-DNA fluorescence (Ripple M O, Henry W F, Rago R P, Wilding G. Prooxidant-antioxidant shift induced by androgen treatment of human prostate carcinoma cells. J Natl Cancer Inst. 1997 Jan. 1; 89(1):40-8). DCFH is oxidized to DCF by ROS to yield easily quantifiable ROS levels monitored by the green fluorescence of the DCF (6-carboxy-2′,7′-dichlorofluorescin diacetate) dye.

The DCF fluorescence in LNCaP cells treated with 1 nM of the androgen analog metribolone was normalized with the blue fluorescence of the Hoechst dye-DNA complex in the same cells at varying concentrations of Mito-Q-C10, in order to evaluate the level the oxidative stress per individual cell.

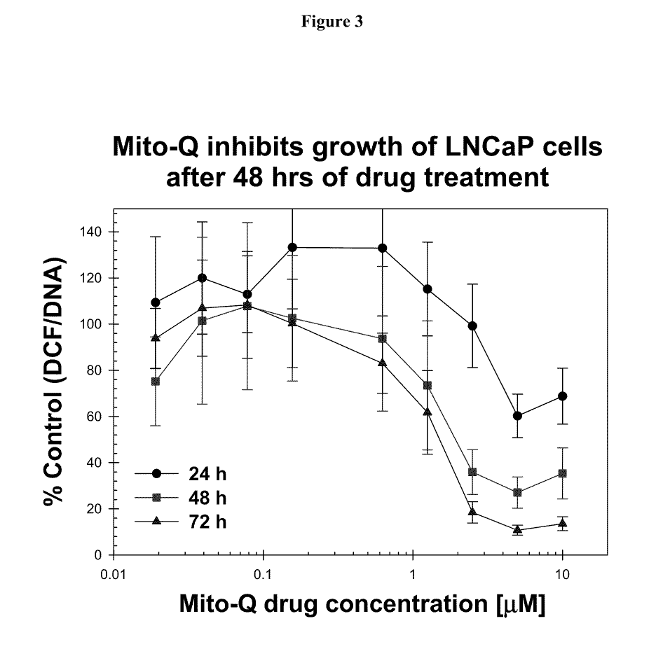

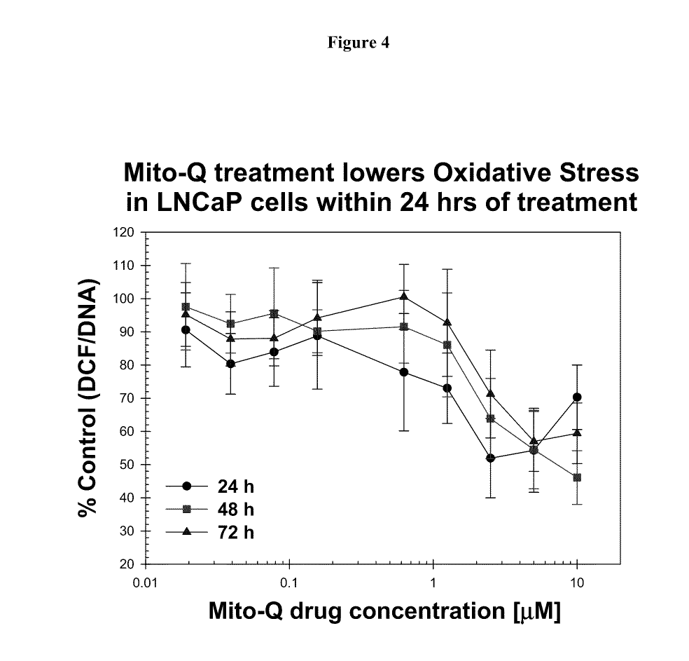

The inhibitory effect of MitoQ-C10 on the oxidative stress level in LNCaP prostate tumor cells can be determined by the ratio of DCF fluorescence/Hoechst dye-DNA fluorescence. MitoQ treatment markedly reduced the oxidative stress in LNCaP cells as determined by DCF fluorescence/DNA fluorescence assay shown in FIG. 3. Mito-Q-C10 treatment effectively and reproducibly reduced the ROS levels in LNCaP cells at concentrations at or above about 1-10 μM. It should be noted that Mito-Q-C10 treatment induced a reduction of oxidative stress determined by DCF assay and mitochondrial function determined by MTT assay, is parallel to Mito-Q-C10's effect in the inhibition of prostate tumor cell growth as determined by DNA assay, as shown in FIG. 4. This oxidative stress is most probably due to increased lipid peroxidation during apoptotic and/or necrotic cell death.

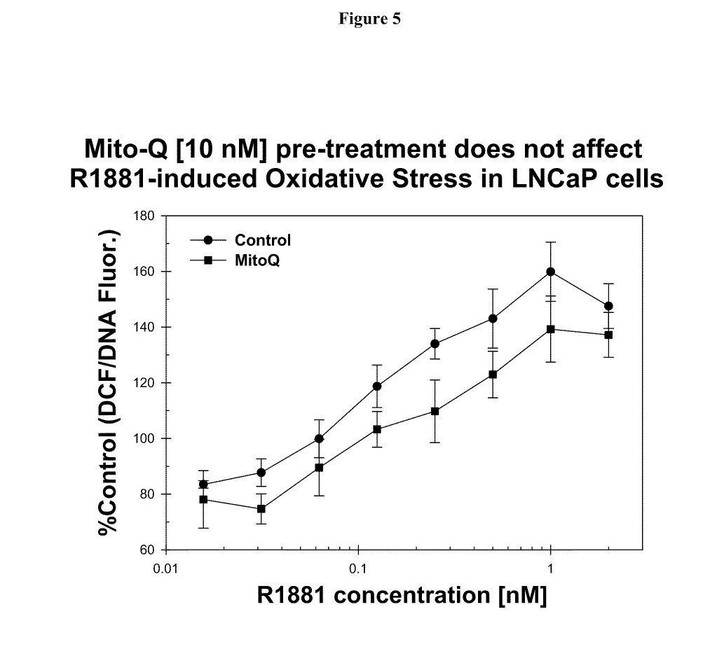

Results shown in FIG. 5 clearly demonstrate that Mito-Q-C10 pretreatment at a sub lethal dose (1 μM) can also completely block the oxidative stress induced by androgen (metribolone) treatment in LNCaP cells. It has been demonstrated that androgen is the leading cause of oxidative stress generation, which is a primary causative agent of prostate cancer and other prostatic diseases, including but not limited, to benign prostatic hyperplasia. Thus, the anti-oxidant effect of Mito-Q-C10 treatment is capable of removing one of the most important metabolic products that causes cancer, cancer progression and cancer metastasis in general and prostate cancer in specific.

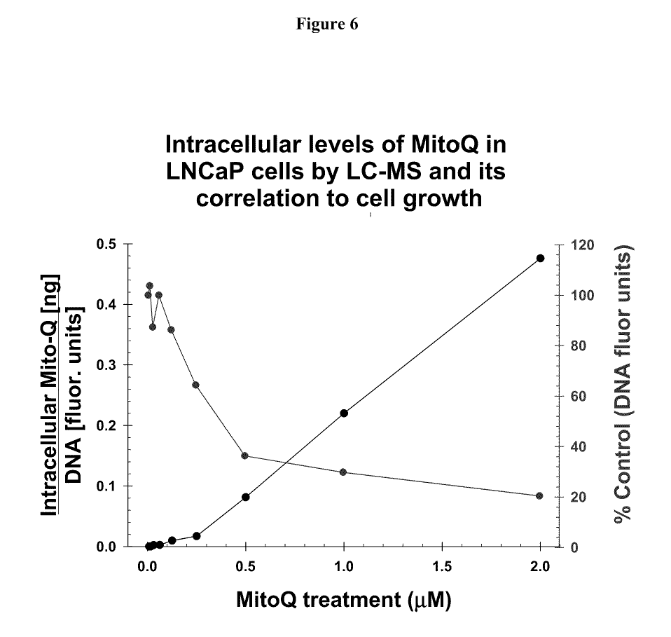

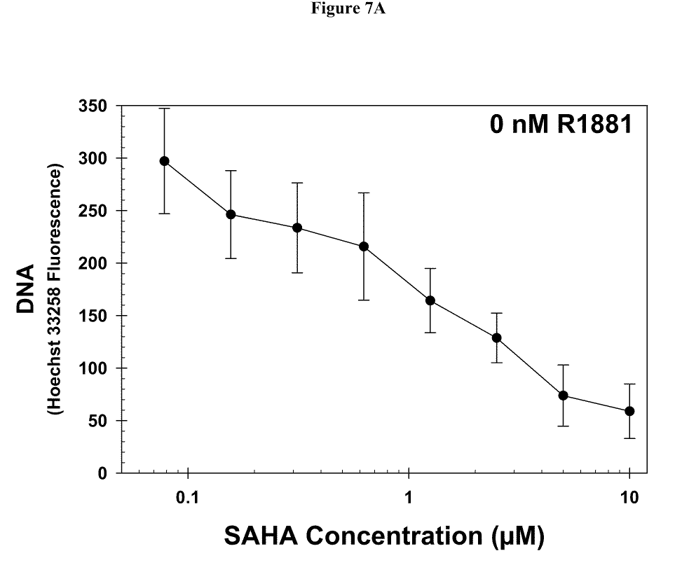

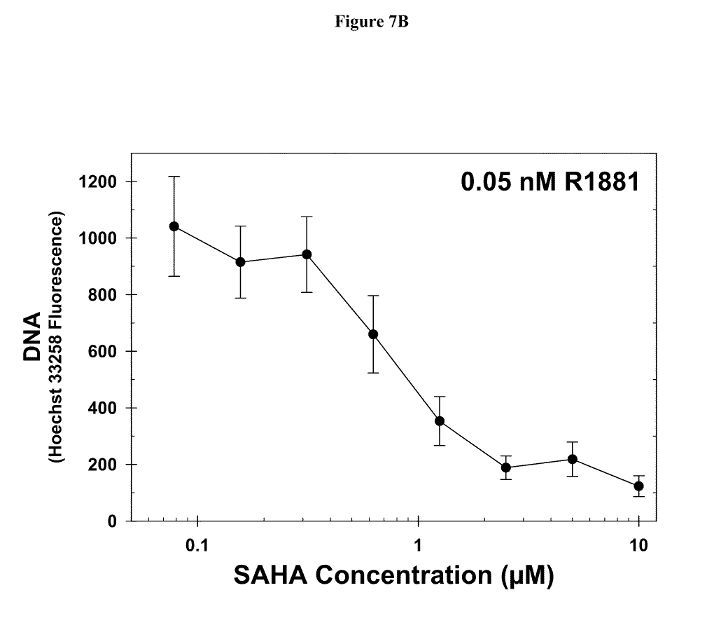

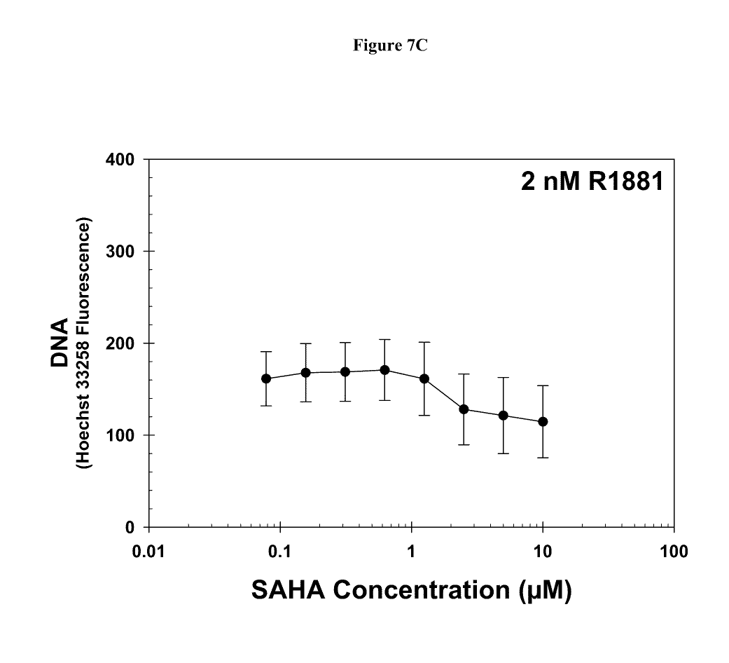

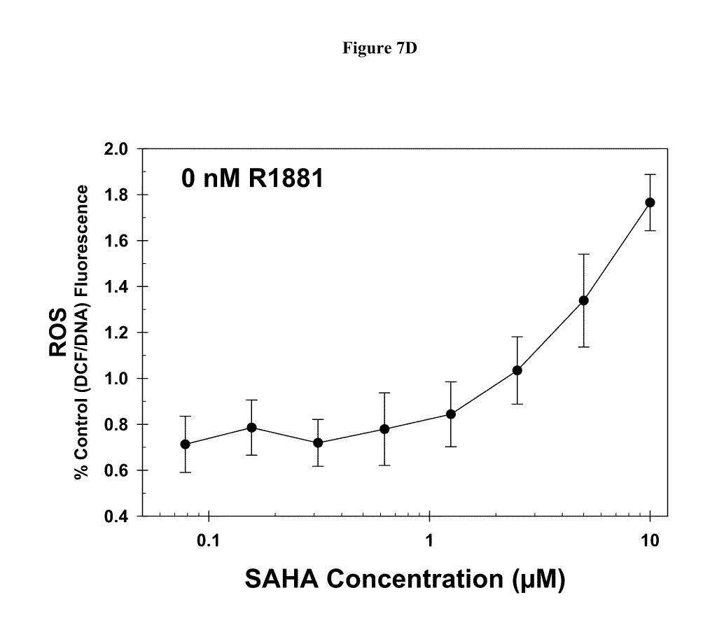

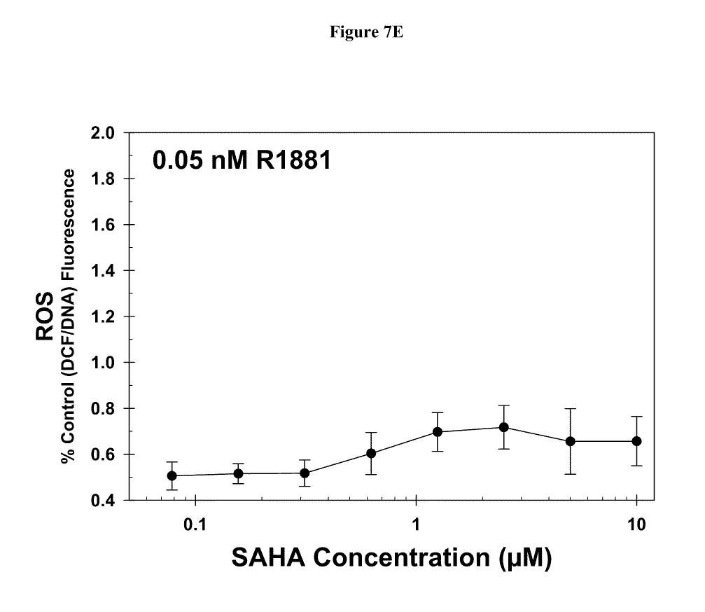

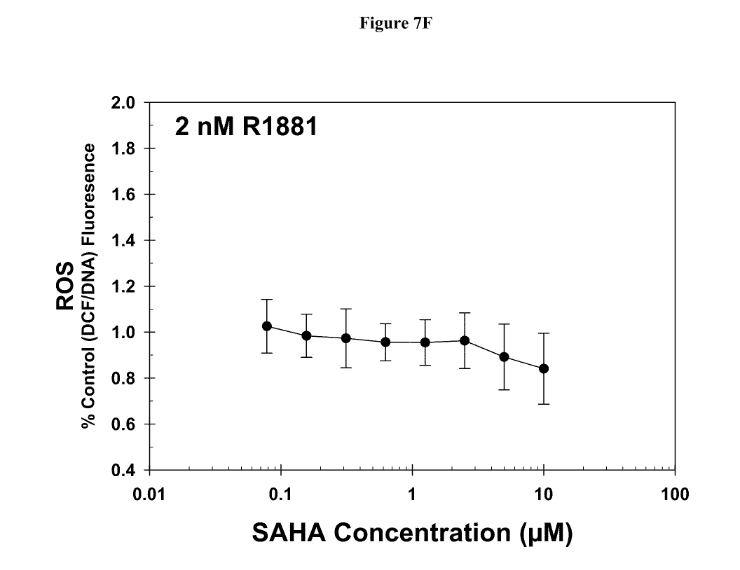

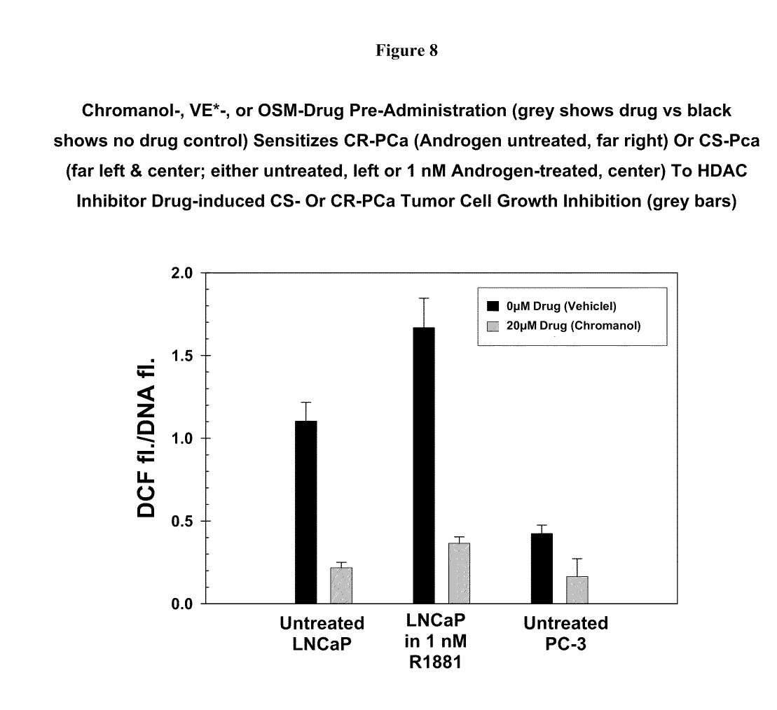

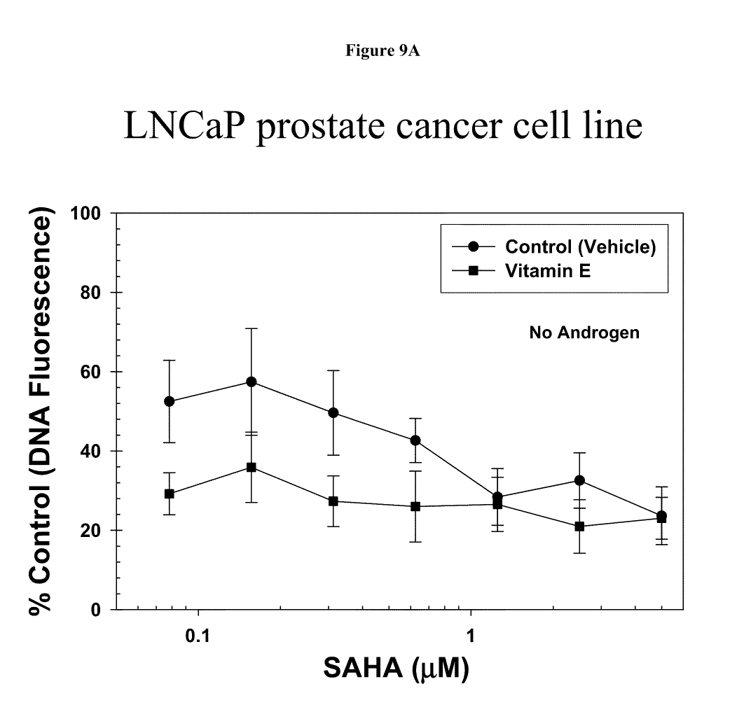

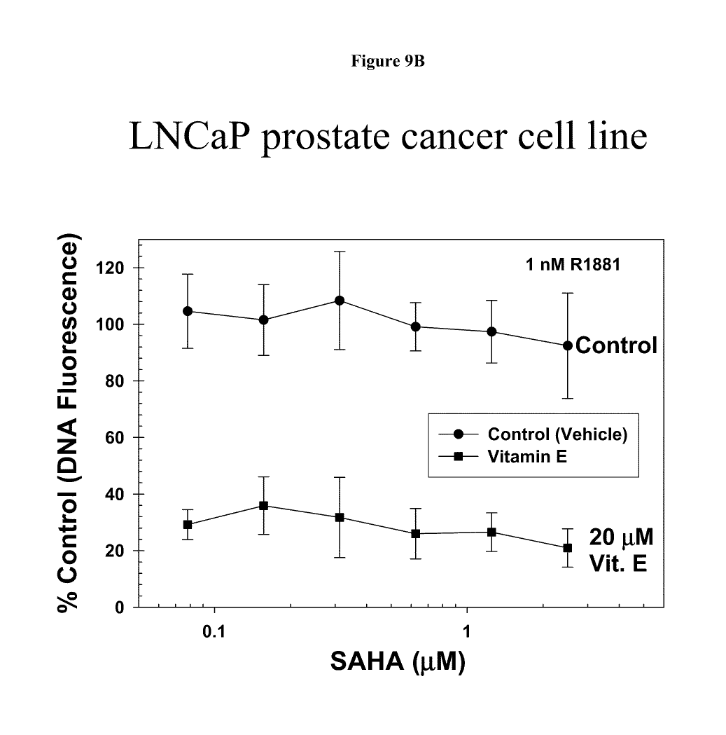

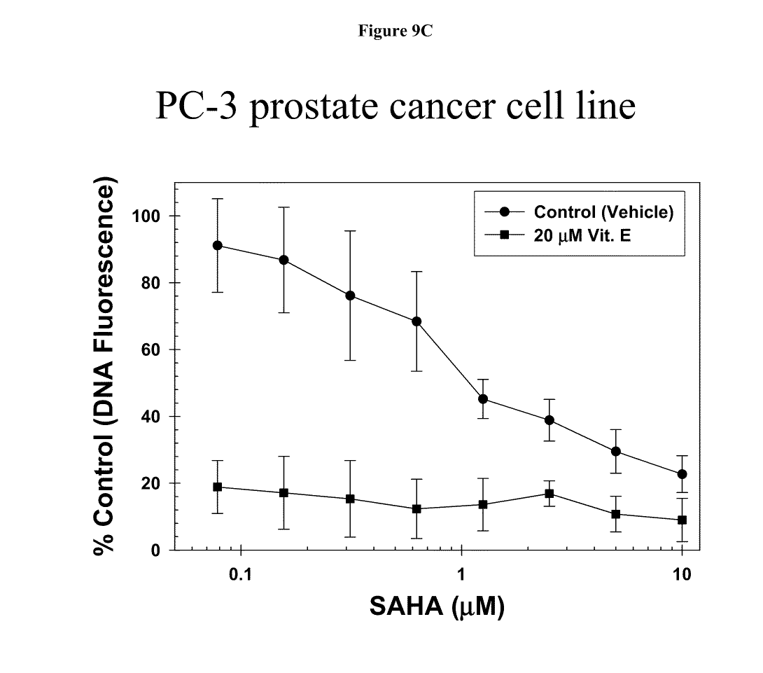

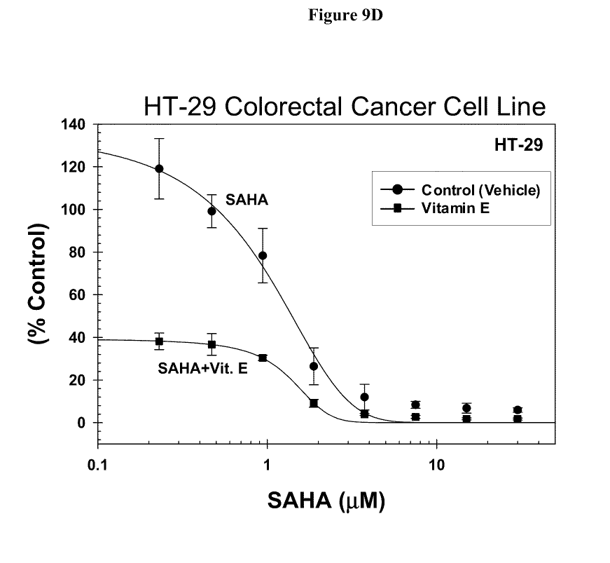

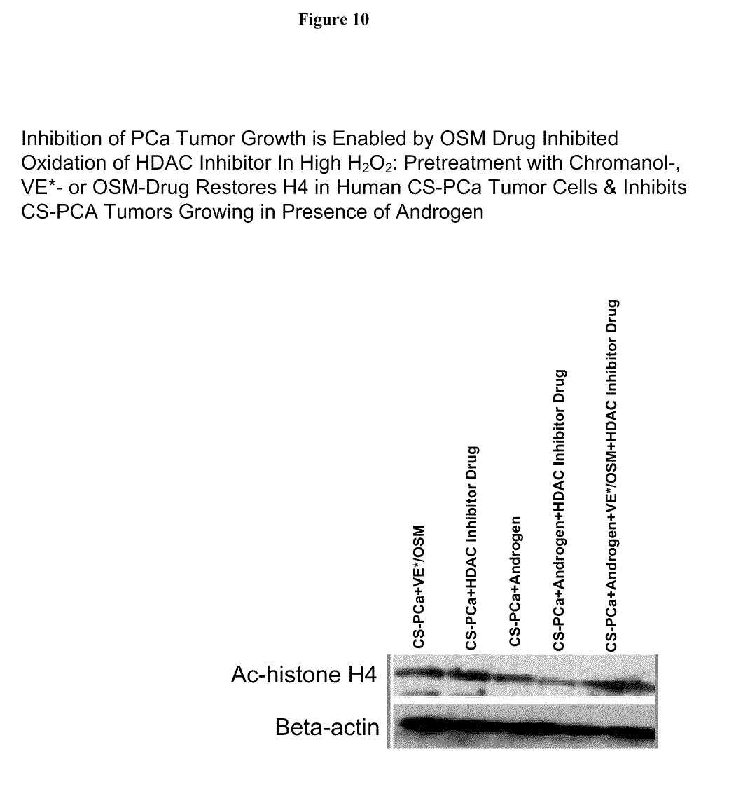

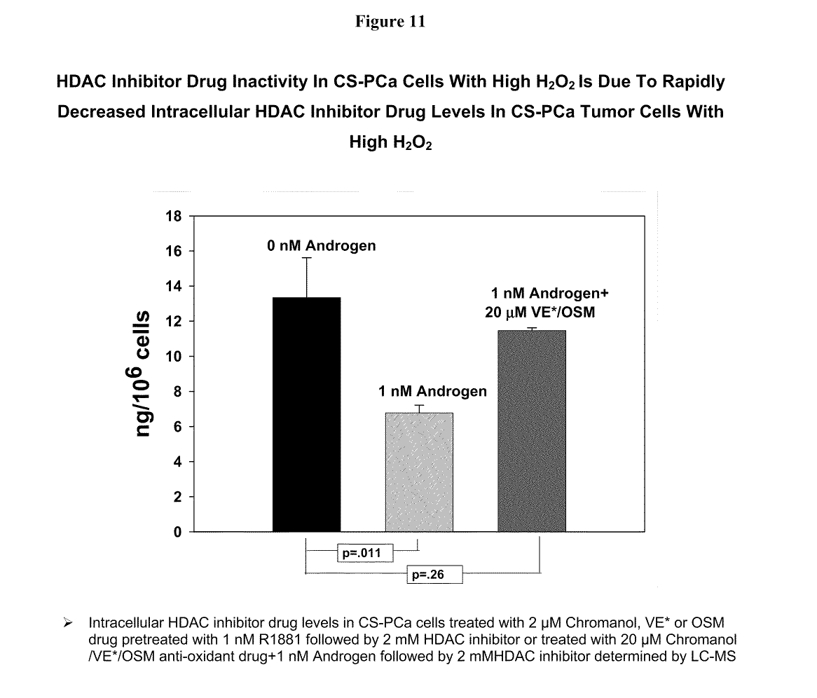

FIG. 6 shows that when prostate cancer cells are treated with Mito-Q-C10, the intracellular level of Mito-Q-C10 is inversely related to cell survival.

Mito-Q-C10 can be safely injected to animals at a dose of 5 mg/kg i.p. At this dose, the serum level of Mito-Q-C10 in the first hour of treatment is 10-20 mg/ml, which is 10-20 fold above the Mito-Q-C10 concentration necessary to block androgen induced oxidative stress in prostate cancer cells. Mito-Q10 is not toxic at 750 nmol (about 20 mg/kg) but toxicity is evident at 1000 nmol (about 27 mg/kg). MitoQ10 is now being developed as a pharmaceutical. For a commercially satisfactory stable formulation it was found beneficial to prepare the compound with the methanesulfonate counteranion and, to facilitate handling, long-term storage, and manufacture, it is adsorbed on to β-cyclodextrin. This preparation was readily made into tablets and has passed through conventional animal toxicity screening with no observable adverse effects at a level of 10.6 mg/kg. The oral bio-availability was determined at approximately 10%, and major metabolites in urine are glucuronides and sulfates of the reduced, hydroquinone form, along with demethylated compounds. In Phase I human trials, MitoQ10 showed good pharmacokinetic behavior with oral dosing at 80 mg (1 mg/kg), resulting in plasma Cmax=33.15 ng/mL and Tmax about 1 hr. This formulation has good pharmaceutical characteristics.

### Example 5a

PMCol not only inhibits the growth of androgen-dependent (LNCaP and LAPC4) as well as androgen-independent (DU-145) human prostate tumor cells in culture, but also inhibits the growth of spontaneous TRAMP mouse tumors. Pharmacokinetic (PK) studies of PMCol in mice administered 100 mg/kg PMCol p.o. or 5 mg/kg of PMCol i.v., using Liquid Chromatography-Mass Spectroscopic (LC-MS) analyses. The data showed that within 15 minutes after oral PMCol administration and 2 hours after i.v. injection, the serum levels of PMCol went down quickly and could not be detected 1 hour after p.o. administration or 4 hours after i.v. administration. Mito-PMCol-C0-1 like Mito-VE-C2 shows no toxicity at 300 nmol intravenously administered at about 4 to about 6 mg/kg. When Mito-PMCol, Mito-PMQ or Mito-PMHQ are administered to mice by intravenous injection, they can be cleared from the plasma and accumulate in the heart, brain, skeletal muscle, liver, prostate and kidney and other organs. These experiments show that once in the bloodstream, the alkylTPP-chromanols and alkylTPP-hydroxylated chromans, Mito-PMCol, Mito-PMQ and Mito-PMHQ or Mito-Tempol compounds, respectively, rapidly redistribute into organs; TPP-derived Mito-PMCol, Mito-PMQ and Mito-PMHQ compounds are orally bioavailable to mice, as was shown by feeding mice tritiated compounds, Administration of Mito-PMCol in the drinking water of rodents, lead to uptake into the plasma and from there into the heart, brain, liver, kidney, and muscle. The Mito-PMCol was shown to be cleared from all organs at a similar rate by a first-order process with a half-life of approximately 1.5 days. Therefore, these studies are consistent with orally administered alkylTPP compounds distributing to all organs owing to their facile permeation through biological membranes.

The inhibitory effect of PMCol on prostate tumor growth is tested in the well characterized TRansgenic Adenocarcinoma of Mouse Prostate (TRAMP) model. A PMCol dose of 100 mg/kg is the MTD of the agents. Tumor development in PMCol treated animals was delayed by over 8 weeks as compared to the control animals.

LC-MS elution profiles for orally administered PMCol, as detected in mouse serum 15 minutes after oral administration, shown a major new peak appears in the plasma as the PMCol peak disappears. This new peak contains an agent that has the molecular ion mass (m⁺/z) of 237, which is identical with reported in the literature (28 and related references therein). This PMCol metabolite remained in the serum for at least 24 hours, which was the last time point of PK studies. We have also reproduced the same retention time and m⁺/z appears when PMCol is oxidized for 12 h at 37° C. These results very strongly indicate that PMCol is oxidized in vivo to the hydroxylated-PMCol. The elution profile and mass fragmentation pattern of hydroxylated-PMCol (PMQ) is similar with that of the major oxidized metabolite. Hydroxylated-PMCol products are reported in the literature and are consistent with the major in vivo metabolite.

In both in culture as well as in vivo PMCol is an active agent and it is further metabolized by oxidation in mammalian tissues and organs. PMCol exhibits significant activity specifically directed against both androgen-dependent and androgen-independent prostate tumor cells.

In order to test the efficacy of PMCol-C2 or Mito-PMCol-C₁₀ in inhibiting growth of prostate tumors in vivo, the PMCol drug formulation was standardized, the route of its administration was determined and determination of the maximum tolerated dose (MTD), when administered orally or by i.v. injection. PMCol or Mito-PMCol-C2 and other analogs can be safely administered to adult tumor bearing mice either par orum (p.o.) in PEG-400 or by intravenous (i.v.) injection in a mixture of ethanol and propylene glycol. Under these conditions, the Maximum Tolerated Doses (MTDs) of PMCol are 100 mg/kg or 7.5 mg/kg for p.o. or i.v., respectively in mice. PMCol has a DLT of 2 grams/kilogram/day given orally every day in rats.

Similar to Mito-Q-C₁₀, Mito-PMCol-C2 and Mito-PMCol-C₁₀ can be directed towards the inner membrane of the mitochondria to block ROS production. In some embodiments, in vitro and in vivo studies for the development of Mito-PMCol molecules as clinically useful CaP chemotherapeutic and chemopreventive agents was determined.

### Example 6

Described herein is the design, recycling with ascorbate, the synthesis of PMCol, (and isomers and analogs), Mito-PMCol, Mito-PMQ, Mito-PMHQ and Mito-PMDHQ and formulations with ascorbate of PMCol and analogs. In some embodiments they are active agents inhibiting CaP cells in culture and for the therapeutic treatment of mammalian prostate tumors in vivo. In other embodiments, the Mito-PMCol based drug is a preventative or therapeutic against prostate cancer. As an adjuvant therapy it may delay or reduce tumor recurrence in individuals who have undergone surgery or radiotherapy for the treatment of their primary prostate tumors. Mito-PMCol can be developed for use as CaP chemopreventive drugs for males at risk. Effective slow and sustained release and other formulations of Mito-PMCol and analogs are in formulations which in some embodiments are conveniently administered to individuals along with pharmacokinetic (PK) data are identified for clinical uses of Mito-PMCol.

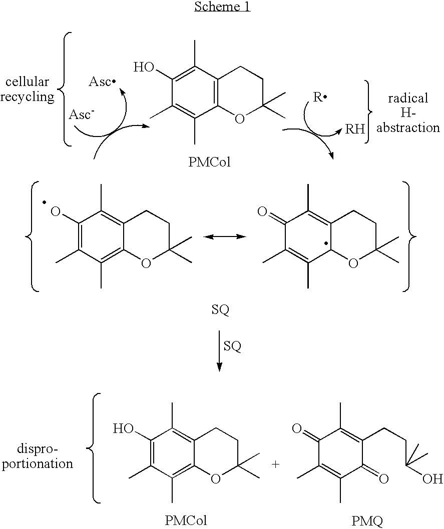

Chemical Synthesis of Mito-PMQ and Mito-PMCol. We describe here the synthesis of derivatives of Mito-PMCol which in some embodiments are potent anti-oxidant and anti-tumor drugs. Analogues of Mito-PMCol also exhibit antioxidant and enhanced anti-oxidant activity by incorporating known substructures that stabilize the PMCol semiquinone radical (SQ) and minimize disproportionation to the quinone, PMQ. In a second approach we design and synthesize and characterize Mito-PMCol analogs to incorporate into improved drug delivery systems that afford to enhance bioavailability and deliver appropriate formulations, salts and concentrations of Mito-PMCol to target areas.

Here we describe the synthesis and tests for new un-targeted or mitochondrial-targeted PMCol analogs with increased anti-oxidant/reducing equivalents, bioavailability and associated therapeutic activities in formulations appropriate for tests in individuals including those for clinical therapeutic and preventative usages. In Scheme 1 above, the anti-oxidant properties of Mito-PMCol derive—from the ability of the dihydroquinone moiety of the chromanol systems to form stable semiquinone radicals (SQ) upon H-atom abstraction by environmental radicals (R.). PMCol and Mito-PMCol can then be recycled by the reaction of the semiquinone radical (SQ) with ascorbate (Asc) or Ubiquinol to undergo further radical scavenging for subsequent radical quenching. However, a competing disproportionation reaction between two semiquinone radicals (SQ) to furnish one molecule of PMCol and one molecule of the quinone PMQ with no anti-oxidant scavenging property is a mechanism for drug deactivation as a radical scavenger. However, PMQ-like Ubiquinone can have activity because of their Quinone based non-scavenging anti-oxidant and mitochondrial oxidative phosphorylation regulating anti-cancer and other therapeutic activities.

Because of disproportionation, one of every two Mito-PMCol molecules is lost. Based upon this mechanism, minimization of the disproportionation increases the lifetime of anti-oxidant scavenging performance of the compound.

### Example 7

The “Mito-twin chromanol and Mito-twin chromanone” (Mito-TwCHol) is identified as a higher order anti-oxidant and reduced anti-oxidant. In some embodiments, Mito-TwCHol anti-oxidant is enhanced in anti-oxidant activity over Mito-PMCol which relates to the stability of the semiquinone radical and its low disproportionation rates. The diminishment in the rate of radical disproportionation is due to increases in the stearic environment introduced by the methylene bridge of the fused PMCol residues. In addition, TwCHol and Mito-TwCHol achieves twice the number reducing equivalents as PMcol and Mito-PMCol, since both the dihydroquinone residues undergo oxidation to the corresponding quinone, Mito-TwCHQ (Scheme 2).

PMCol has bioavailability in blood serum (oral PMCol, 4 mg/kg; iv PMCol, 0.5 mg/kg), and a serum half-life (oral PMCol, 0.5 hr.; iv PMCol, 2.0 hr.). Also, the concentrations required for PMCol in vivo for its anti-cancer and other therapeutic activities at the oral PMCol MTD of 100 mg/kg can be reduced with Mito-PMCol administration, likely due to its ability to achieve a significantly increased intracellular mitochondrial concentration within a relatively short time-frame, following administration to an individual. The observed acid (pH 2.0) lability of PMCol is consistent with the bioavailability of PMCol, when administered orally. Mito-PMCol, like PMCol, is metabolized and may be rapidly oxidized/hydroxylated. The Mito-PMCol oxidized metabolite is the ring-opened Mito-PMQ, similar to the PMCol oxidized metabolite as the ring-opened PMQ.

The LC-MS peak corresponding to PMQ appears in serum within minutes after oral administration and persists in the blood serum. Mito-PMCol, Mito-TwCHol and Mito-PMCol dimer analogs, like their non-conjugated forms, in some embodiments, become sequestered in the mitochondrial inner membranes. PMCol in prostate shows increased cellular absorption and retention in cytoplasmic mitochondria. Mito-PMCol in other embodiments are more rapidly incorporated and at higher concentrations,

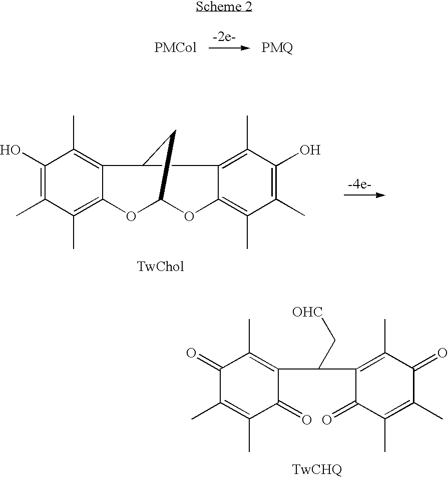

The anti-oxidant and anti-cancer and other therapeutic activities of PMCol analogs in other embodiments are increased by increasing the bioavailability, serum stability and increasing absorption by mammalian organs and tissues, and also by analogs of PMCol described above. Clinically relevant and conveniently prepared new pharmaceutical formulations containing superior anti-oxidant Mito-chromanols, including the novel Mito-twin PMCol and Mito-PMCol dimer. The non-targeted form is referred to in its non-Mito form as, 1,3,4,8,9,11-Hexamethyl-6,12-methano-12H-dibenzo[d,g][1,3]dioxocin-2,10-diol (referred to here as TwCol or Twin-chromanol or TwCol). Mito-TwCHol can deliver two times as many reducing equivalents as Mito-PMCol and improve bioenergetic and biochemical parameters in mitochondria exposed to oxidative stress, as monitored in mitochondria in cell free extracts. Mito-TwCHol, in further embodiments, without toxicity at concentrations of up to 50 nmol of TwCHol/mg mitochondrial protein. Mito-TwCol is therapeutically active in human cells in vitro and mammals in vivo. Mito-TwCol sterically protects the radical and localizes the radical over more centers to shut down radical disproportionation and increases Mito-TwCol anti-oxidant, anti-cancer, and other therapeutic activities. Mito-PMCol in yet further embodiments, has anti-tumor therapeutic activity with both androgen-dependent and androgen-independent human prostate tumor cells, and with human tumor xenografts growing in nude mice as well as spontaneous prostate tumors.

### Example 8

### Synthesis and Structure-Activity of Mito-Twin Chromanol and Other Derivatives

Mito-TwCHol is synthesized by modification of the literature procedures for TwCHol (Scheme 3). In addition, structure-activity studies on Mito-TwCHol elucidate the source of the diminished rate of disproportionation. TwCHol analogues are synthesized to evaluate the role of the methylene bridge on overall radical stability and antioxidant activity. As illustrated in Scheme 3, the analogues I-III are in some embodiments prepared by condensation of 2,3,5-trimethyl-1,4-dihydroquinone with the appropriate dicarbonyl compound or diacetal.

The twin chromanol analogue II has been previously reported in the literature as an intermediate for polymer synthesis and other industrial applications. All three analogues I-III possess 4 reducing equivalents similar to TwCHol and Mito-TWCHol.

### Synthesis and Structure-Activity Studies of Mito-PMCol and Mito-PMQ and Linked-Dimers.

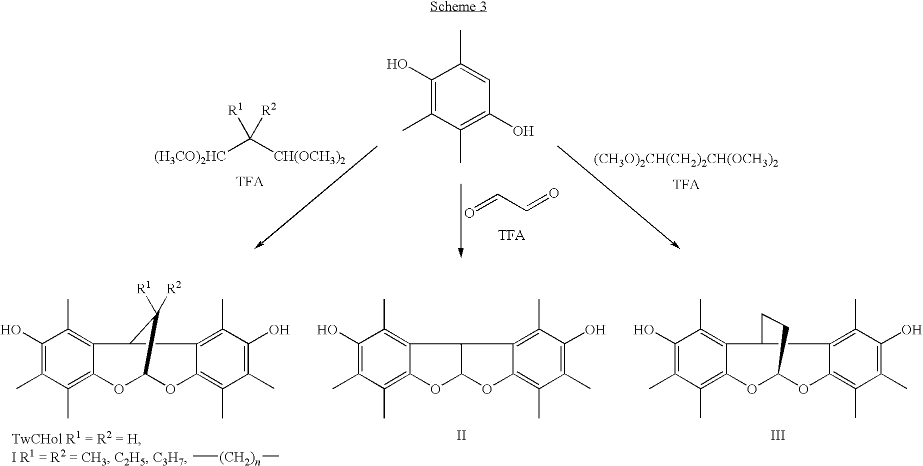

To explore further the effect of enhanced antioxidant activity observed for the TwCHol, two novel series of dimeric PMCol, Mito-PMCol and Mito-PMQ and derivatives were prepared. Like the TwCHol, the target compounds can possess twice the reducing equivalents as PMCol. However the intermediate semiquinone radicals can exhibit greater stability as a result of greater resonance stabilization imparted by entire Mito-PMCol-dimer system. The first class of the PMCol dimer derivatives V-X can possess a vinyl-linking group between the two PMCol moieties. The vinyl linker can serve as a conduit for resonance stabilization of the semiquinone radicals by both PMCol units. This provides a stabilizing effect and reduces the potential for disproportionation. The synthesis of the PMCol-dimers proceeds from the readily available hydroxymethyl PMCol derivatives. Both the symmetrical dimers (same PMCol substitution on each monomer unit) and the asymmetrical dimers (different PMCol substitution on the monomer unit) can be prepared. An example of synthesis of the asymmetrical PMCol dimer VIII is illustrated in Scheme 4.

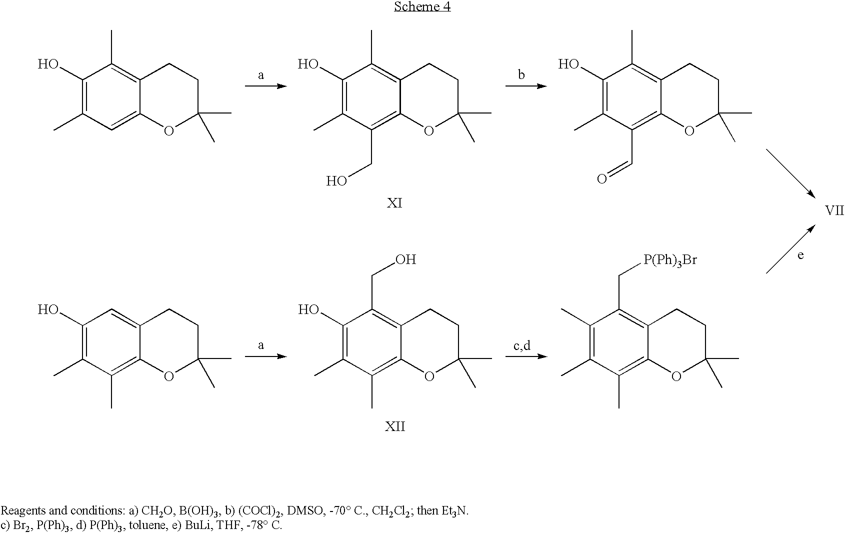

The 8-hydroxymethyl and 5-hydroxymethyl derivatives, XI and XII, respectively are prepared in a straightforward fashion from the readily available 6-hydroxychromanols using modifications of literature procedures. The 5-hydroxymethyl derivative XII reconverted into the phosphonium salt by bromination with concomitant treatment with triphenylphosphine in toluene. The 8-hydroxymethyl derivative XI are converted into the aldehyde by Swern oxidation. Wittig olefination of the aldehyde with the phosphorus ylide of XII affords the desired PMCol dimer VIII and Mito-PMCol dimer. The trans-isomer is a major product. However, the cis-isomer is, in some embodiments, obtained and has antioxidant activity.

### Synthesis and Structure-Activity Studies of Fused PMCol-Dimers and Mito-PMCol-Dimers.

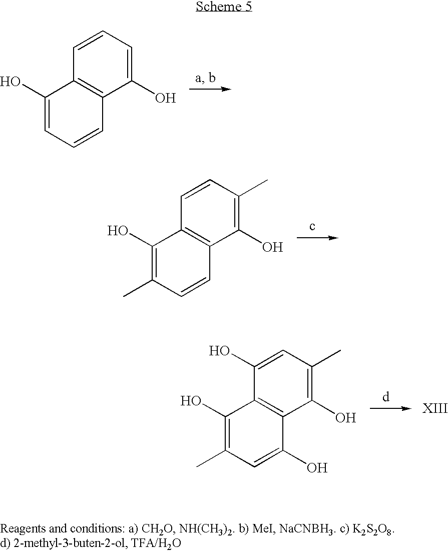

A series of fused Mito-PMCol dimers were also prepared as antioxidants. The fused PMCol-dimer analogues XIII and XIV exhibit greater radical stability than TwCHol because of the greater resonance stabilization afforded the semiquinone radical by the fused aromatic system. In addition, these fused-dimers possess the same number of reducing equivalents (four equivalents) as TwCHol and the vinyl-linked PMCol derivatives.

As illustrated in Scheme 5, the synthesis of XIII is achieved from commercially available 1,5-dihydroxy-naphthalene. Ortho-methylation followed by Elb's oxidation furnishing the desired fused-dihydroquinone. Treatment of the fused-dihydroquinone with 2-methyl-3-buten-2-ol in trifluoroacetic acid/water affords the fused dimer XIII in good yields. The synthesis of the XIV is achieved in similar fashion from the corresponding 1,5-dihydroxyanthrace.

Additional structure-activity studies focuses on the benzochromanol (XV) and naphthochromanol (XVI) congeners of Mito-PMCol. The antioxidant activity of PMCol is significantly increased when fused into an aromatic ring system. The benzochromanol Vitamin K₁-chromanol has been reported to exhibit greater anti-oxidant activity that α-tocopherol (Vitamin E). XV can be a better anti-oxidant than PMCol. Although compounds XV and XVI possess the same number of reducing equivalents as PMCol, the stability of the semiquinone radical is increased due to the extended conjugation of the fused aromatic system. This leads to decreased disproportionation rates and longer duration of activity. In addition, the substitution of the benzochromanol (XV) and naphthochromanol (XVI) ring systems allows for the electronic optimization of the dihydroquinone for maximum anti-oxidant efficiency. As illustrated in Scheme 6, the benzochromanol (XV) and naphthochromanol (XVI) Mito-PMCol congeners are prepared from the corresponding 1.4-naphthyldihydroquinone and 1,4-anthryldihydroquinone, respectively. Although the benzochromanol (XV) has been reported, the anti-oxidant activity has not been previously evaluated biologically and reported in the scientific literature.

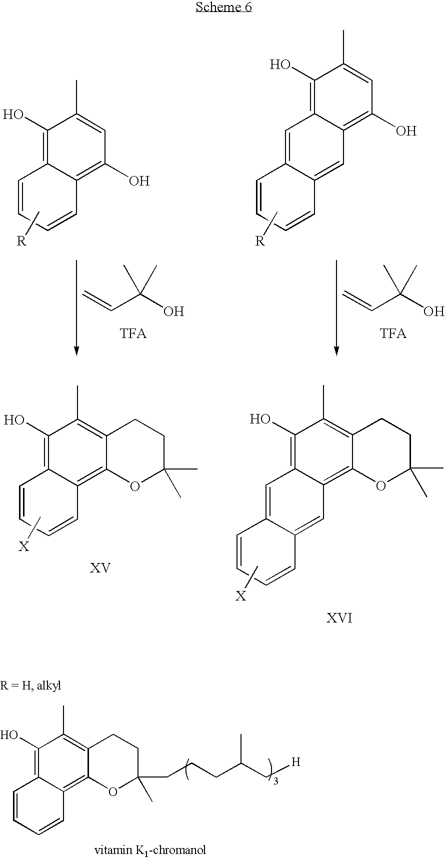

### Example 9

### Synthesis and Activity in Studies of PMCol poly-(L-glutamate) and Mito-PMCol poly-(L-glutamate)

The potency and efficacy was increased in sustaining PMCol activities is measured by preparing monomer units of Mito-PMCol having functionality for the preparation of blood serum esterase activated PMCol pro-drug system or for the coupling to a drug delivery scaffold. The hydroxymethyl-PMCol analogs XI, XII and XVII are readily synthesized by hydroxy-methylation of the corresponding 6-hydroxychromanol derivatives (see Scheme 4). The hydroxyl moiety serve as a point of attachment for an ester containing pro-drug (succinate) or to the macromolecular delivery system (polyglutamate). Administration of the Mito-PMCol in this form can lead to a greater concentrations of Mito-PMCol at the tumor or other cells without significant increases in dosage. The aminomethyl-PMCol analogs XVIIIa-c are synthesized to provide amide PMCol-. These compounds are prepared by aminomethylation of the corresponding 6-hydroxychromanol derivatives or by oxidation and reductive amination of the corresponding alcohols. The amino XVIIIa-c derivatives offer the advantage that they can also be converted into the acid salts (HCl, citric acid) that offer better solubility in aqueous media and provide enhanced bioavailability.

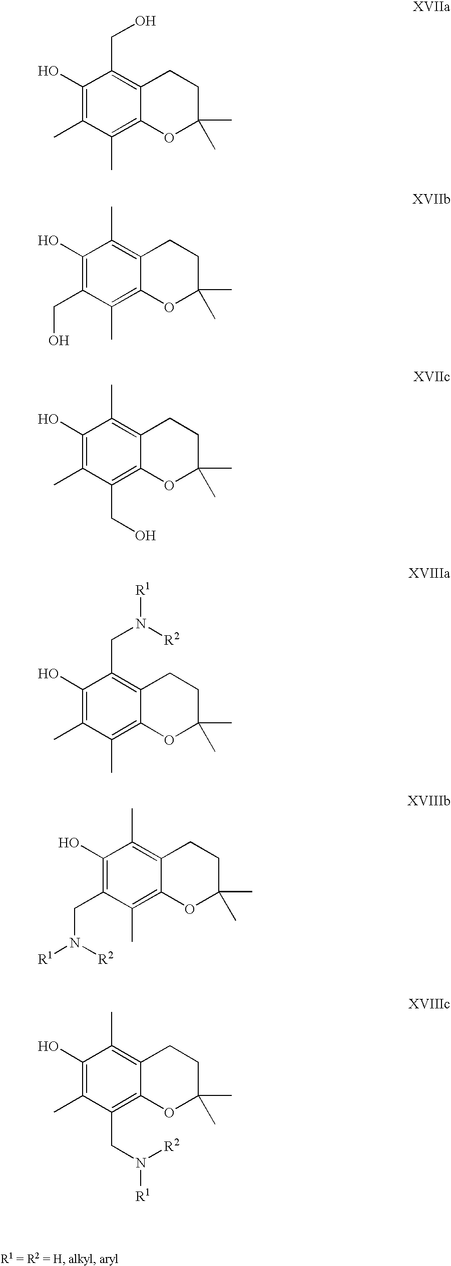

The alcohol and amino derivatives of Mito-PMCol that exhibit potent anti-oxidant activity are investigated in a macromolecular drug delivery system. Active alcohol PMCol derivatives XVII as well as Mito-PMCol are attached to a poly-(L-glutamate) scaffold via an ester linkage between the carboxyl residue of the polymer backbone and the phenol of PMCol or the hydroxyl group of analogues XVII (Scheme 7).

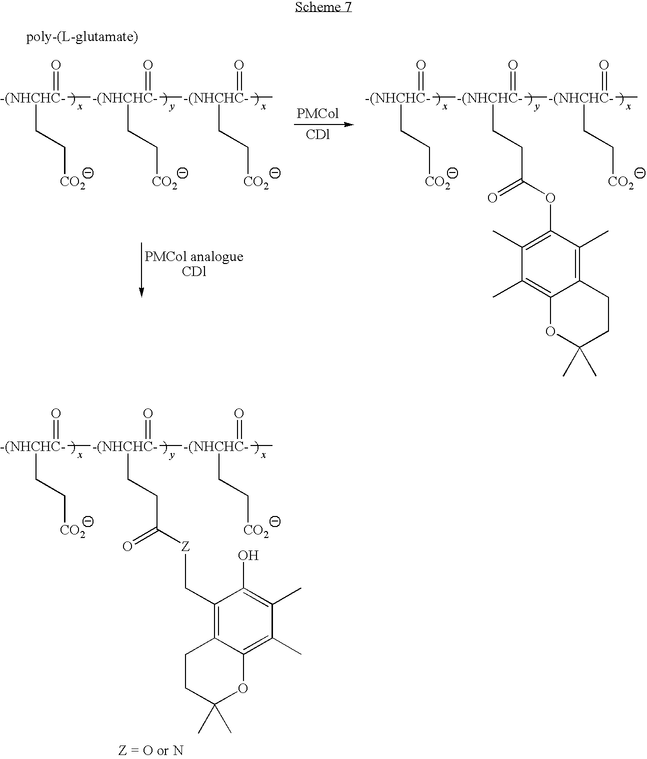

Alternatively, active amine analogues XVIII are in some embodiments attached to poly-(L-glutamate) via an amide linkage between the carboxyl residue of the polymer back bone and the amino group (Scheme 7). The poly-(L-glutamate) has been reported to be a useful scaffold for drug delivery. The carboxylate moiety is sufficiently removed from the polypeptide backbone so as not to sterically inhibit the chemistry of the attached drug. In addition, the unbound carboxylate residues provide for good aqueous solubility for the polypeptide-drug complex. The water-soluble poly-(L-glutamate)-PMCol-Mito-T system is introduced into the blood serum where serum esterase enzymatically causes hydrolysis of the ester or amide bonds and releases the drug. The poly-(L-glutamate) scaffold is then subsequently metabolized into non-toxic L-glutamic acid. The poly-(L-glutamate)-PMCol system is prepared according to the literature. The Mito-PMCol loading of poly-(L-glutamate) is measured by complete hydrolysis of the polypeptide ester linkages followed by HPLC analysis for PMCol or PMCol analogues.

### Example 10

Oxidation and NO products of PMCol:

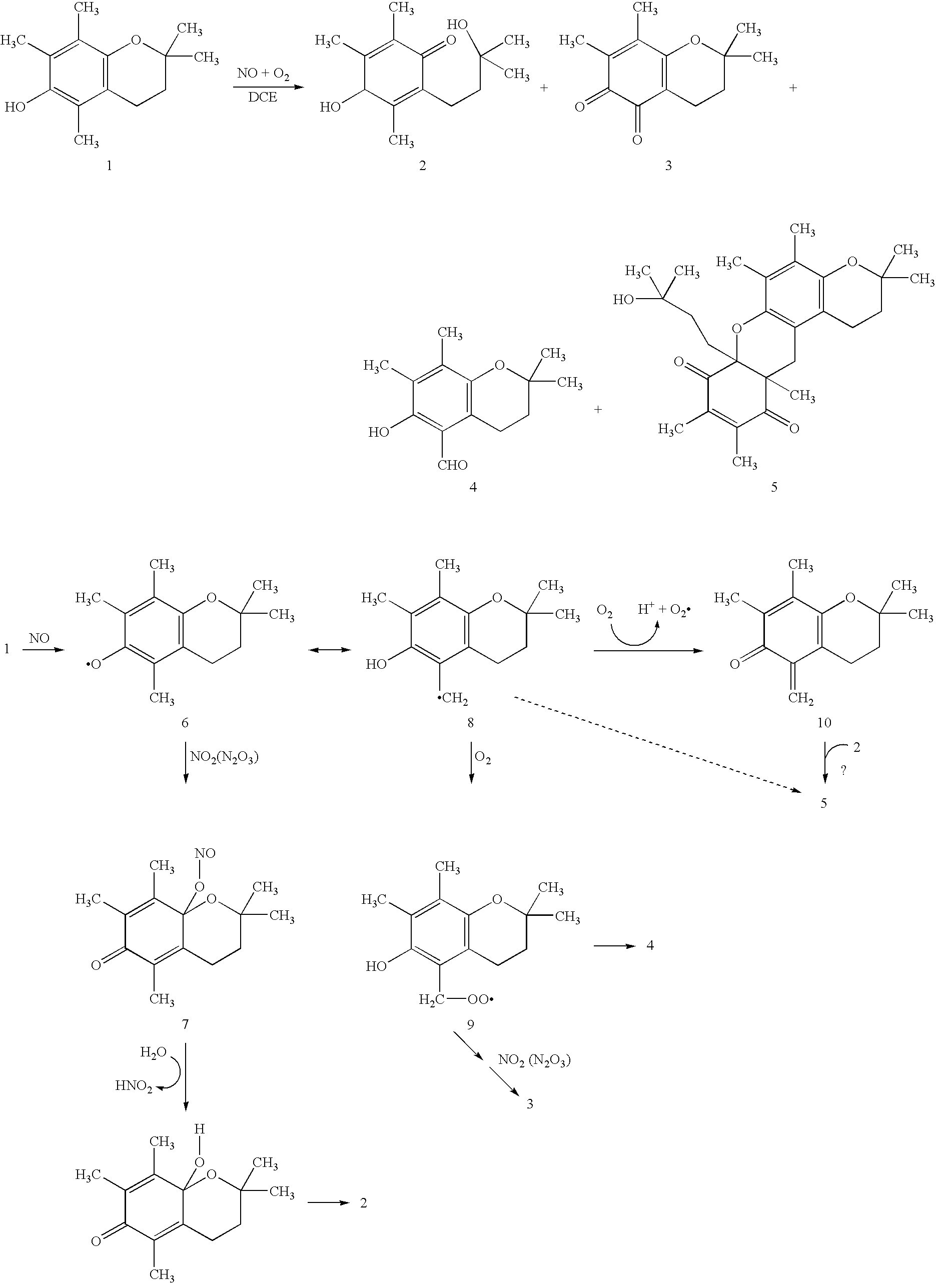

α-Tocopherol (α-Toc, ATCol, Vitamin E, VE) is a ubiquitous antioxidant in biological systems and protects biological molecules from the oxidation induced by various kinds of active oxygens. Its action is derived from the quenching of active oxidants with one electron reduction and the radical chain reaction is terminated by this process. Nitric oxide (NO) is one of the most important biological radical molecules and has been known as mediator in many physiological phenomena. In addition, NO brings about cytotoxic activity when it is generated in relatively high concentration, and reacts with molecular oxygen or superoxide to give dinitrogen trioxide (N₂O₃), nitrogen dioxide (NO₂), or peroxynitrite. These higher nitrogen oxides (NOx) are known to have high reactivity and oxidation activity in spite of the slight reactivity of NO itself. These active species derived from NO are give oxidative damages to the body and can interact with α-Toc, which is one of the major antioxidants in biological systems. In order to simplify the analysis of the reaction mixture, a known α-Toc analogue, 2,2,5,7,8-pentamethyl-6-chromanol (PMC), is also a substrate. It was found that high yields of products were obtained by controlling the amount and ratio of NO and O₂, and that the products distribution was varied by the ratio and mixing time of two gases. When the reaction was carried out using PMC 1 and an equimolar amount of NO in air in dichloroethane (DCE),2-(3-hydroxy-3-methylbutyl)-3,5,6-trimethyl-1,4-benzo-quinone (PMQuinone, PMQ) (2) was obtained. Two major products were obtained whose structures were assigned as 2 and 2,2,7,8-tetramethylchroman-5,6-dione (PMCred). Among the other minor products, two compounds were identified as 5-formyl-2,2,7,8-tetra-methyl-6-chromanol and 2,3-dihydro-3,3,5,6,9,10,11a-heptamethyl-7a-(3-hydroxy-3-methylbutyl)-1H-pyr-ano [2,3-a]xanthene-8(7aH),11(11aH)-dione. All the reactions were carried out three times, and the reaction yields shown are mean values. The reaction seldom proceeded by the mixing of PMC and 10 equiv of NO in the absence of O₂, thus there seems to exist no interaction between PMC and NO. In the case of 1 equiv of NO, however, about a half amount of PMC was consumed accompanied by formation of a small amount of 2. The reason for these phenomena was attributed to a slight contamination of oxygen in the experiment of entry 1, in which the inner pressure was lower than that of entry 5. Product distribution varied when PMC and NO were allowed to stir for 2 h before the addition of O₂. The results indicate that the non-productive interaction exists between PMC and NO in the absence of O₂, as suggested in the literature. When 1 or 2 equiv of NO was used, PMC was consumed in the presence of 0.5 equiv of O₂ to give almost equimolar amounts of 2 and 3 and the yields became higher with lesser amount of NO. In these cases, the timing of O₂ addition brought about a large effect on the products yields, which also suggests the direct interaction between NO and PMC in the absence of O₂. By decreasing the NO amount, it is necessary to make the reaction time longer, but the use of excess amount of O₂ resulted in the considerable consumption of PMC. In this case, the minor products 4 and 5 were obtained more than in the cases under the former conditions. For the comparison of the reactivity, 1 equiv of NO₂ was used instead of NO and O₂. In short reaction time (10 min), 2 was obtained in 41% yield without considerable formation of 3, and the yield of 3 gradually increased with the elongation of the reaction time. Although the reaction with NO₂ corresponds to the reaction with NO and 0.5 equiv of O₂ from the viewpoint of the stoichiometry, the results were different as shown in entries 14 and 10. Thus these also suggested that the formation of NO₂ was incomplete in the mixture of NO and 0.5 equiv of O₂. This yields four oxidation products of PMC by the reaction with NO in the presence of various amounts of oxygen.

Since the overall product yields were obtained at up to 90%, the results are thought to afford the rational background for the total reaction mechanism. Although there must be several pathways to give these products, one of the supposed reaction mechanisms is as shown in Scheme 2. It is well known that NO reacts with O₂ to form N₂O₃ or NO₂ according to the ratio of NO/O₂. Thus, based on the stoichiometry, the major reactive species in the reaction are regarded as NO₂ (+N₂O₃)+little O₂, N₂O₃ (+NO), NO₂ and NO₂+O₂, respectively, although these reactive species interconvert with each other in the reaction mixture.

NO interacts with PMC without the aid of O₂, thus NO must have the reactivity toward PMC to give the phenoxy radical. In the presence of reactive NO₂ (or N₂O₃), 6 was supposed to be further oxidized by NO₂ (or N₂O₃) to form PMQuinone 2.

When active NOx was decreased, this process must become slower, and oxygen can substitute for NOx to oxidize 6, and the reaction pathway is supposed to change into the formation of PMCred 3 or 4. When the amount of NOx was lowered further, the oxidation might proceed via the sole participation of oxygen after the initial formation of 6. Since 5 was thought to be a product of Diels-Alder reaction of a quinonoid 10 and 2, the reaction was carried out in the presence of excess 2, but the yield of 5 was not increased. Therefore, there must be an alternative pathway to the formation for 5 other than the one shown in Scheme 2. Even in the presence of 0.25 equiv of NO, PMC was consumed by excess O₂ and elongation of the reaction time. These data suggest there is a pathway where NO₂ might act in a catalytic manner for the oxidation. The similar results were reported by Kochi et al. that hydroquinone was oxidized by catalytic amounts of NO₂ in the presence of excess amount of oxygen. PMC and NO in the presence of various amounts of oxygen to form the products, four of which were identified and quantified. The oxidized products were obtained in good yields by the restriction of the amounts of NO and oxygen. In addition, the product distribution was altered by the change of NO/O₂ ratio. Experiments showed that the reaction with alpha-tocopherol gave analogous results to those presented here.

### Example 12

Numerous different human cancer cells are relatively more oxidatively stressed than are normal cells. Cellular high oxidative stress in prostate tumor cells was hypothesized to be responsible for the loss of growth inhibitory activity of HDAC inhibitor drugs. The reduction of the high oxidative stress in particular human cancer cell lines and human primary tumors, was accomplished by pretreatment with a dietary or pharmaceutical anti-oxidant, including a lipid soluble/water insoluble Vitamin E formulation or using pharmaceutical drugs which are water soluble Vitamin E analogs including chromanols, quinones, modified quinines, plastoquinones, tetracyclenes, tempols, or other anti-oxidant drugs. We tested the therapeutic effectiveness of these anti-oxidant compounds for their abilities and utilities in therapeutically sensitizing the cancer cell lines and primary human and animal tumors to HDAC inhibitors, including SAHA, as well as other oxidation sensitive anti-inflammatory drugs, prostate and other known cancer chemoprentative or cancer chemotherapeutic drugs. Human CaP cells LNCaP and PC-3, colon cancer cells HT-29 and HCT-115, lung cancer cells A549 and NCI-H460 and breast cancer cell MDA-MB231 were from the American Type Culture Collection (Manassas, Va.). The LNCaP cells are maintained in humidified air containing 5% CO₂ at 37° C. in 10 cm diameter tissue culture plates in Dulbecco's modified Eagle medium (DMEM) supplemented with 5% heat-inactivated fetal bovine serum (FBS) and 1% 100× antibiotic, antimycotic solution (F5 medium). PC-3 cells were maintained in DMEM containing 5% FBS. All other cell lines were cultured in RPMI-1640 medium containing 10% FBS. For Androgen Deprivation the LNCaP cells used in all experiments were cultured in F5 medium and transferred to “low” androgen conditions in DMEM containing 4% charcoal stripped FBS (CSS) plus 1% non-stripped FBS (F1/C4 medium). In previous studies, this medium showed sufficient androgen depletion, but no adverse growth effects related to nutrient depletion. Two days after transfer, cells were trypsinized, counted and seeded in F1/C4. The day after seeding, cells were treated with specific concentrations of an androgen analog R1881, which is widely used as a surrogate for androgen in cell culture conditions. Treated cells were incubated for another 24 hours in humidified air containing 5% CO₂ at 37° C. before the addition of SAHA. Graded concentrations of an anti-oxidant or a HDAC inhibitor, such as SAHA were added to the cells a day after androgen addition or two days after seeding (for control cells) in F1/C4 medium. Depending on the experiment, the test drug was added by serial dilution to 96-well tissue culture plates or at calculated concentrations to 10 cm tissue culture plates. After addition, cells were incubated for 3 days in humidified air containing 5% CO₂ at 37° C. in preparation for various assays. At the end of incubation, cells in 96-well plates were assayed for total ROS production in live cells with 2′,7′-dichlorofluorescein diacetate (DCF) dye (Molecular Probes, Inc., Eugene, Oreg.) following a published protocols. Wells were washed with 200 μL of Kreb Ringer (KR) Buffer pre-warmed to 37° C. In every well, 100 μL DCF in pre-warmed KR Buffer were added to a final concentration of 20.5 μM. Cells were incubated in humidified air containing 5% CO₂ at 37° C. for 45 minutes and then read in a fluorescence plate scanner set at 480 nm excitation/530 nm emission to measure DCF dye fluorescence. After scanning, the plates were stored at −80° C. in preparation for the DNA assay.

For DNA Assay the test cells seeded in 96-well tissue culture plates that were previously used in the DCF assay were thawed at room temperature. Hoechst dye (33258) was prepared in 0.05 M Tris (pH 7.5), 2 M NaCl, 1 mM ethylenediamine-tetraacetate (high salt TNE) to make a final stock dye concentration of 10 μg/ml following a published procedure. Each well received 200 μL of the Hoechst-TNE stock. Each 96-well tissue culture plate was measured for total fluorescence of Hoechst dye in a fluorescence plate scanner set at 360 nm excitation/460 nm emission to measure DCF dye fluorescence.

For sample preparation and cellular HDAC inhibitor drug (i.e. SAHA) measurements by LC-MS cells were trypsinized, counted, pelleted, washed once with PBS, dried and pellets were stored below −70° C. The day of the experiment, pellets were incubated in ice for 5 min in 100 μL lysis buffer (0.25 M sucrose, 0.06 M KCl, 0.05 M NaCl, 0.01 M 2-(N-morpholino) ethanesulfonic acid (MES), 0.01 M MgCl₂, 0.001 M CaCl₂, 0.0001 M phenyl-methyl-sulfonyl-fluoride (PMSF), 1 mM EDTA and 0.2% Triton X-100 (pH 6.5). Ten volumes chilled 99.5% acetonitrile, 0.5% acetic acid was added to all lysates, vortexed vigorously and incubated in ice for another 5 minutes for SAHA to be extracted into the organic solvent. Tubes were centrifuged at 5,000 g for 5 minutes, and a calculated volume of the organic layer (generally 80% of the total organic solvent added) was aspirated carefully from the top. The organic solvent was dehydrated under a flow of nitrogen, redissolved in 50 μL 99.5% acetonitrile, 0.5% acetic acid. Ten μL of each extract was used for LC-MS analysis, and the assay was repeated three times. All data were normalized to the total volume of cell extract and expressed as ng SAHA/10⁶ cells.

For chromatography of SAHA levels in LNCaP cells was determined by a modification of a published LC-MS method of determining SAHA in patient serum. The LC-MS system consisted of an Agilent (Palo Alto, Calif.) 1100 auto sampler and binary pump, Agilent 1100 column thermostat and an Agilent Zorbax 300SB-C18 column (3.5 μM, 2.1×100 mm). The mobile phase solvent A was acetonitrile and acetic acid (99.5%:0.5% v/v) and solvent B was water and acetic acid (99.5%:0.5% v/v). The solvent gradient and the flow rates were adjusted appropriately. A 5 minute post-run column wash at 10% solvent A, 90% solvent B was maintained at 0.2 ml/min. The column thermostat was maintained at 25° C. for the complete run.

The Mass detector for the mass detection was carried out with Agilent 1100 quadruple moment bench-top mass spectrometer with electrospray ionization in the positive ion mode at 3000 V. For both the single ion MS and scanning MS/MS mode, the desolvation temperature was 340° C. with the drying gas flow rate of 12 l/min at a nebular pressure of 40 psig. The scan mode was between 150 to 300 m⁺/z and the single ion detection (SIM) modes were set at 265.2, 232.2 and 172.2 m⁺/z. All data were collected, stored and analyzed using Agilent software for data collection, peak detection and integration.

For the construction of LNCaP clones stably transfected with siSSAT the clones were created following published procedures. Briefly, oligonucleotides for silencing SSAT were designed based on the published sequence. The annealed oligonucleotides were inserted into pSF1 vector (SBI; System Biosciences, Mountain View, Calif.). LNCaP cells stably expressing pSIF-H1-siSSAT vector were established using a lentiviral system. The silencing of SSAT in these cells was verified by qRT-PCR.

For HDAC assays a high throughput HDAC assay was standardized using a Biomol (Plymouth Meeting, Pa.) HDAC assay kit with minor modifications of the manufacturer supplied protocol. Briefly, at the end of the drug treatment, media in the 96-well assay plates were dumped and cells were washed once with 25% PBS and then allowed to swell in 30 μL deionized double distilled water for 1 hour at room temperature. Plates were then frozen at or below −70° C. The day of the experiment, the plates were thawed at 4° C. for 30 minutes. Fifteen μL of the cell lysates were transferred to 96-well white round bottom plates, mixed thoroughly with 10 μL HDAC assay buffer (50 mM Tris-HCl, 137 mM NaCl, 2.7 mM KCl, 1 mM MgCl₂, pH 8.0) and 25 μL manufacturer supplied fluorescence tagged HDAC substrate (KI-104, Biomol Inc.) appropriately diluted in the same HDAC assay buffer. The plates were incubated at 37° C. for 30 minutes. The reaction was stopped with a manufacturer supplied Developer solution (Developer I, 20×, Biomol Inc.) containing 200 μM trichostatin A (TSA), and the plates were read within an hour at 360 nm excitation/460 nm emission in a Saphire (Tecan US, Inc., Durham, N.C.) multimode plate reader using 150 mV Photomultiplier voltage setting. The remaining 15 μL of the cell lysates were used for DNA assay using 85 μL deionized double distilled water and 200 μL Hoechst 33258 dye following DNA assay protocol described above. All DNA fluorescence data were multiplied by a factor of two in order to determine the DNA reading of the total cell lysates.

For Western blot analysis of acetylated histones the total cellular histones were isolated following a published procedure. Prior to gel loading, pH was adjusted to 7.2 with 1 M NaOH. A 10 μl aliquot from each sample was set aside for protein estimation. The rest of the samples were loaded and electrophoresis done in SDS-PAGE. Western blot analysis was carried out following a published procedures using anti-acetyl H4 antibody (Millipore, Temecula, Calif.). β-actin was used as control for protein loading. The acetyl histone H4 band intensities were calculated and normalized to β-actin intensities.

LNCaP human prostate cancer cells are pretreated with two concentrations of androgen analog metribolone which either decreases or increases cellular reactive oxygen species (ROS), followed by a treatment with graded concentrations of SAHA. 96-well plate-based DNA and dichlorfluorescein-diacetate (DCF-DA) fluorescence assays are used to determine cell growth and total cellular ROS, respectively. Liquid-Chromatography-Mass-Spectrometry (LC-MS) method is used to measure the intracellular SAHA levels in metribolone pretreated or untreated control LNCaP cells. The cell growth inhibitory activity of SAHA directed against human prostate and colorectal cancer cells with high ROS levels and in other lung cancer cells with low ROS levels are also determined in cells pretreated with a sub-toxic doses of antioxidant test agents that reduced cellular ROS.

Histone deacetylase (HDAC) is a class of enzymes present primarily in the nucleus that de-acetylates histones H3 and H4. HDAC activity prevents expression of genes that are required for cell cycle arrest and to induce apoptosis. Therefore, HDAC inhibition arrests cell proliferation and causes apoptosis, cellular differentiation and/or senescence. Suberoylanilide Hydroxamic Acid (SAHA) is a HDAC inhibitor that causes arrest of cell proliferation and cell death. It has undergone advanced clinical trials against lymphoma and was approved for the treatment of cutaneous T-cell lymphoma (CTCL). SAHA, however, is inactive against human prostate, breast, colon and other cancers.

LNCaP is an androgen-responsive human CaP cell line that was established in the early '80s from a metastatic lesion in the lymph node of a CaP patients. In 1997, Ripple et al. first reported that, in LNCaP cells, treatment with graded concentrations of R1881, an androgen analog, generates varying levels of reactive oxygen species (ROS) such as superoxide, hydroxyl radical, hydrogen peroxide, etc. as determined by DCF dye oxidation assay. When treated with R1881 concentrations less than 0.1 nM, “low androgen,” LNCaP cells showed significantly lower cellular ROS as compared to treatment with 1-10 nM R1881, “normal to high androgen.” However, within the 1-10 nM R1881 concentration, no significant difference was observed in the amount of LNCaP cell growth or ROS generation. In addition to LNCaP cells, other human prostate, colon and some breast cancer cells also have high ROS levels; whereas, human lung cancer cells are remarkably low in cellular ROS.

Although SAHA has been successful in the treatment of CTCL lymphoma, multiple clinical trials have failed to show efficacy of SAHA against prostate, colon, breast and other types of human malignancies. There can be several reasons for cellular resistance to SAHA, e.g.; (i) SAHA may kill cells by inducing oxidative stress. Compared to cells with low oxidative stress such as CTCL lymphoma cells, other cancers with tumor cells with adaptations to high oxidative stress can be unaffected by drugs that can induce cell kill by a MOA inducing oxidative stress; (ii) high superoxide dismutase (SOD) enzyme activity in these cells may neutralize oxidative stress produced by SAHA and thus, inhibit its activity; (iii) SAHA may be oxidized by the high levels of ROS produced in the prostate, colon or breast cancer cells and thereby, require high drug concentrations that are not clinically achievable.

We discovered that the inactivity of SAHA against CaP cells with high ROS is not due to changes in SOD activity or due to intrinsic cellular resistance to ROS, but rather is due to a rapid decrease in intracellular SAHA concentrations in cells with high ROS levels. Reduction of ROS levels by silencing a major enzyme in ROS producing pathway activates SAHA against CaP cells. Reducing cellular ROS by pretreatment with an anti-oxidant such as lipid soluble/water insoluble Vitamin E or water soluble analogs, chromals and other OSM drugs also may synergistically increases SAHA sensitivity of CaP, colon and breast cancer cells, but not that of certain cancer cells that have low intrinsic ROS. Thus HDAC inhibitor drugs like SAHA or other oxidation sensitive chemotherapeutic drugs in combination with anti-oxidants is a therapeutic treatment for various different cancers with high oxidative stress, including those tumors with high rates of hydrogen peroxide production that are totally unresponsive to SAHA or these other oxidation sensitive drugs as single agents.

### SAHA Inhibits Growth of Prostate Cancer Cells Only at Low Oxidative Stress.

Fluorescence readings of Hoechst dye (Hoechst 33258) complex with DNA in the nuclei of cancer cell lines are proportional to the number of cells present in each well. DNA fluorescence of LNCaP cells after pre-treatment with R1881 followed by increasing concentration of SAHA from 0-10 μM is shown in FIG. 1a. In LNCaP cells pretreated with no R1881 and 0.05 nM R1881, cell growth was inhibited almost linearly with a logarithmic increase in SAHA concentration (FIGS. 1a.A & 1a.B, respectively). In LNCaP cells pretreated with 2 nM R1881, however, SAHA has negligible effect on cell growth at all concentrations tested (FIG. 1a.C). The growth inhibitory effect of SAHA at a concentration at or above 1 μM in cells treated with no androgen or with 0.05 nM R1881 is markedly more pronounced than is the growth inhibitory effect of equivalent concentration of SAHA in cells pretreated with 2 nM R1881. These data suggest that LNCaP cells exposed to normal serum androgen (2 nM) are relatively resistant to growth inhibitory effect of SAHA as compared to cells growing at low or no androgen.

### Growth Inhibitory Effect of SAHA is not Dependent on Cellular Oxidative Stress in Prostate Cancer Cells.

Fluorescence of oxidized DCF dye is proportional to the total cellular ROS. When DCF fluorescence is normalized with the DNA fluorescence from the same well of the 96-well plate, the ratio DCF fluorescence:DNA fluorescence is proportional to the ROS generated per cell. The plots of the ratio of DCF/DNA fluorescence of LNCaP cells with or without pretreatment with various R1881 concentrations vs. increasing SAHA concentrations are presented in FIG. 1b. In LNCaP cells pretreated with no R1881, ROS increases with an increase in SAHA concentration (FIG. 1b.A). In LNCaP cells pretreated with 0.05 nM and 2 nM R1881, however, increase in SAHA concentration has negligible effect on total cellular ROS levels Total cellular ROS levels at all SAHA concentrations are higher in cells treated with 2 nM R1881 than in cells treated with 0.05 nM R1881.

Effect of SAHA Against siSSAT LNCaP Cells.

Spermidine/spermine acetyl transferase (SSAT) is a major enzyme in androgen-induced ROS production in LNCaP cells. We constructed a LNCaP cell clone stably transfected with siRNA against SSAT (siSSAT) that reduces SSAT expression by >90%. R1881 treatment has no significant effect on ROS production in siSSAT clone as compared to a marked increase in LNCaP cells transfected with the control vector containing scrambled sequence. Growth inhibitory effects of SAHA on 2 nM R1881 pretreated vector control and siSSAT cells are expressed as % control of DNA fluorescence of corresponding cells treated with appropriate concentrations of R1881, but not treated with SAHA. The growth inhibitory effect of SAHA is significantly pronounced in 2 nM R1881 siSSAT cells as compared to what observed for the vector control cells.

Effect of SAHA on HDAC Activity in the siSSAT Clone.

Next, we determined the effect of graded concentrations of SAHA on the HDAC activities in vector control and siSSAT cell lines. The HDAC activity is expressed as a ratio of HDAC product fluorescence/DNA fluorescence in relative fluorescence unit (FU). All data were normalized to the same ratio in corresponding cells growing under identical conditions (with or without R1881), but not treated with SAHA. In cells not treated with androgen, SAHA has nearly similar efficiency in inhibiting HDAC activity in both vector control and siSSAT cell lines. At concentrations >1 μM, however, SAHA does not inhibit HDAC activity in R1881 pretreated vector. control cells, but inhibits HDAC activity in R1881 to similar extent as in R1881 untreated siSSAT cells. The HDAC inhibitory effect of SAHA parallels the ability of SAHA in arresting growth of androgen-treated siSSAT cells and not the growth of androgen-treated vector control cells.

### Effect of SAHA in Vitamin E Pre-Treated Cells.

Based on these results, we hypothesize that the high cellular ROS is responsible for the deactivation of SAHA in prostate cancer cells. Therefore, we tested whether or nor pre-treatment of cells with an anti-oxidant that is known to reduce cellular ROS levels should sensitize the cells to SAHA. We pretreated prostate cancer cells LNCaP (both treated and untreated with R1881) and PC-3, colon cancer cells HT-29, breast cancer cells MDA-MB231 and lung cancer cells A549 and NCI-H460 cells with α̅Tocopherol succinate (Vitamin E). For LNCaP cells treated with R1881, Vitamin E was added right before R1881 addition to neutralize any excess ROS production due to androgen treatment. Effects of 96 hour treatment with graded concentrations of Vitamin E on cell growth was determined separately. From that study, Vitamin E concentrations that are non-toxic to each cell line were selected for pretreatment. Treatment with a non-toxic dose of Vitamin E (20 μM) on the ROS levels of LNCaP (treated and untreated with R1881) and PC-3 prostate cancer cells are shown in FIG. 3. Vitamin E treatment markedly reduces the ROS levels in LNCaP and PC-3 cells. Similar reduction of cellular ROS by Vitamin E has been observed in oxidatively stressed breast and colon cancer cells. Due to the very low level of oxidative stress in these human lung cancer cells, the effect of Vitamin E treatment on the ROS levels in these cells could not be accurately determined.

The effects of SAHA on the growth of Vitamin E pretreated and untreated human cancer cells are shown in FIG. 4. All data are normalized as % control of DNA fluorescence of corresponding cells treated with Vitamin E alone. Both androgen-untreated and -treated LNCaP cells (FIGS. 4A and 4B, respectively) as well as PC-3 cells (FIG. 4C) become markedly sensitive to growth inhibition by SAHA after pretreatment with a non-toxic dose of 20 μM Vitamin E that reduces cellular oxidative stress. SAHA sensitivity of HT-29 and MDA-MB231 cells are also higher in Vitamin E pretreated cells, as compared to Vitamin E untreated cells. The increase in sensitivity is synergistic as determined by using the formalism developed by Chou and Talalay. It is noted that there is a marked difference in growth inhibitory effect of SAHA against these cell lines at clinically achievable SAHA dose of 1 μM. The lung cancer cells A549 and NCI-H460 with low ROS levels, however, do not show any appreciable increase in SAHA sensitivity after Vitamin E pretreatment at any concentration of SAHA.

### Effect of Vitamin E Pretreatment on SAHA Induced Changes in Acetyl Histone Levels.

Western blot analysis of acetyl histone levels in LNCaP cells treated with 20 μM Vitamin E alone, 1 nM R1881 alone and 2 μM SAHA alone, along with a combination of R1881+SAHA and Vitamin E+R1881+SAHA, using anti-acetyl H4 antibody has been performed. Western blot of β-actin is used to control for protein loading. A representative western blot is shown in FIG. 5. Vitamin E and R1881 has little effect on the acetyl-histone H4 level. SAHA treatment causes a small, but significant increase in the acetyl-histone level that shows that SAHA inhibits HDAC activity in LNCaP cells growing in the absence of androgen. There is a marked decrease in acetyl-histone H4 level in R1881 pretreated cells, suggesting an appreciable loss of HDAC inhibitory activity of SAHA in these cells. Pretreatment with Vitamin E almost completely restores the acetyl histone H4 level in R1881 treated cells, showing a restoration of HDAC inhibitory activity of SAHA in Vitamin E treated cells.

### LC-MS Estimation of Intracellular SAHA Concentration.

Using the procedure standardized during this study SAHA is detected as a single peak in LNCaP cell extracts spiked with increasing concentrations of SAHA. Cellular SAHA concentrations in LNCaP cells were measured as ng SAHA/10⁶ cells using a standard curve for SAHA generated using LNCaP cell extracts spiked with calculated amounts of SAHA. SAHA concentrations in cells treated with 5 μM SAHA for 24 hours either untreated or pretreated with 1 nM R1881 were measured. Within 24 hours, the SAHA level in LNCaP cells pretreated with R1881 is less than half of that in R1881 untreated cells. In Vitamin E pretreated cells, however, there is no significant decrease in intracellular SAHA level, at least in the first 24 hours.

The data show that SAHA is inactive specifically against cancer cells with high oxidative stress probably due to oxidative degradation of SAHA in these cells. A reduction of oxidative stress in these cells by Vitamin E pretreatment sensitizes the otherwise SAHA resistant cancer cells with high oxidative stress to the growth inhibitory activity of SAHA. In LNCaP cells treated with no androgen (F1/C4 medium) or low androgen (0.05 nM R1881), DNA fluorescence, which is a measure for cell growth, decreases almost linearly with a logarithmic increase in SAHA concentration. Thus, SAHA inhibits LNCaP prostate cancer cell growth, when functioning at low androgen conditions (≦0.05 nM R1881) with IC₅₀<1 μM. In LNCaP cells growing in normal androgen level (1 nM R1881), however, there is little effect on cell growth even at 10 μM SAHA (FIG. 1a.C). R1881 at 0.05 nM R1881 has growth stimulatory and at 1 nM or above concentration exhibits growth inhibitory effect on LNCaP cells. This is reflected on the total DNA fluorescence values at very low SAHA concentration. The changes in DNA fluorescence with increasing SAHA concentrations clearly demonstrate that SAHA inhibits growth of LNCaP cells grown in a medium with low androgen (0 nM and 0.05 nM R1881), but not in a medium with high androgen (1 nM R1881).

To test if changes in ROS have effects on the growth inhibitory activities of SAHA, cellular ROS levels are compared with cell growth under low and high androgen conditions. In LNCaP cells growing in the absence of androgen (F1/C4 medium), cellular ROS levels increase as cell growth decreases, supporting the published observation that SAHA treatment increases cellular ROS levels, which was hypothesized to be one of the reasons for the cell growth inhibition by SAHA. In LNCaP cells, growing at 0.05 nM R1881, however, very similar growth inhibition has been observed without any appreciable increase in ROS levels. On the other hand, LNCaP cells with high intrinsic ROS levels growing in the presence of normal androgen conditions (1 nM R1881) are resistant to SAHA. These and other similar data indicate that the growth inhibitory effects of SAHA is not due to an increase in cellular ROS levels in SAHA treated cells. The results also show that LNCaP human prostate cancer cells are not intrinsically resistant to the growth inhibitory effects of SAHA and exhibit SAHA resistance only when grown at normal serum androgen levels. As androgen-dependent cells are mainly found in patients with normal serum androgen levels at an early stage of CaP recurrence, most early stage prostate cancer patients will not respond to SAHA at the serum SAHA level of ˜349 ng/mL (˜1.3 μM) for patients given clinically approved oral SAHA dose of 400 mg qd. On the other hand, androgen-resistant CaP cells such as PC-3 are intrinsically resistant to SAHA below 10 μM. Thus, advanced prostate cancer in patients with low serum androgen levels will also not to respond to SAHA. It may be possible to treat CaP patients with SAHA either at an early or a late stage of the disease.

SAHA may affect superoxide dismutase (SOD) enzyme activity differently in the presence of androgen, causing changes in the amount of ROS and thereby, indirectly affecting cytoplasmic ROS levels at high androgen conditions. However, the SOD assay data show that there is no significant difference in the SOD activity of LNCaP cells that have been pretreated with 0.05 nM or 1 nM R1881 prior to treatment with 10 μM SAHA. These and other similar results rule out the possibility that androgen induced changes in SOD activity are responsible for altering cellular oxidative stress and therefore, SAHA sensitivity of cells growing at different androgen concentrations.

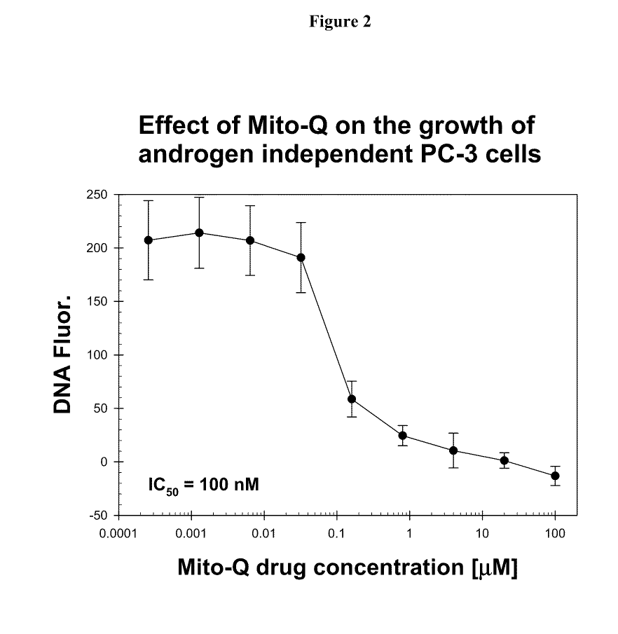

In the siSSAT LNCaP clones that are unable to produce ROS upon androgen treatment, SAHA has marked growth inhibitory effect in high androgen treated cells. The effect is similar to that of SAHA against LNCaP cells growing at low androgen concentration. We have also determined that the cellular HDAC activity is very similar in LNCaP cells either transfected with the siSSAT vector or a control vector with scrambled sequence. HDAC activity in vector control cells pretreated with 1 nM R1881 and then treated with increasing concentrations of SAHA increases after an initial decrease. HDAC activity in R1881 untreated vector control cells as well as androgen-treated and untreated siSSAT cells decreases in a similar fashion (FIGS. 2b.A and 2b.B). This anomalous increase in HDAC activity in androgen-treated vector control LNCaP cells is possibly due to a loss of SAHA activity in these cells. Since both these cell lines are derived from the same parental LNCaP cells, effect on SAHA uptake, excretion, changes in chromatin structure, etc. are expected to remain the same in both cell lines and therefore, can be ruled out as possibilities for the differential activity of SAHA in these two cell lines. Thus, an oxidation of intracellular SAHA in high ROS containing CaP cells is the major reason for the loss of SAHA activity against human CaP cells.

A mechanism other than HDAC inhibition for the growth inhibitory activity of SAHA has been considered. The possibility of changes in cellular polyamine levels in siSSAT cells altering the chromatin structure and thus, modifying SAHA activity is a possibility. There are, however, only minor changes in cellular polyamine levels between vector control and siSSAT cell lines. Thus, the possibility of cellular polyamines that may affect chromatin structure and thus, altering SAHA sensitivity of the siSSAT cells is ruled out.

Based on these results, oxidative loss of SAHA in high ROS containing cells is the major cause of loss of SAHA activity against these cells. Thus, a reduction of cellular ROS by pretreatment with an anti-oxidant such as lipid soluble/water insoluble Vitamin E or water soluble VE analogs can activate SAHA against human cancer cells with high ROS levels.

We have studied the growth inhibitory effect of SAHA on human prostate, colon and breast cancer cells with high oxidative stress and lung cancer cells with low oxidative stress with or without pretreatment with an anti-oxidant Vitamin E. The optimum concentrations were determined for Vitamin E or water soluble Chromanol-based analog required for reducing ROS levels in each of these cell lines without any growth inhibitory or cytotoxic effect of Vitamin E or the water soluble chromanol. As these human lung cancer cells have very low ROS levels, the effect of Vitamin E on the ROS levels of these cells, if any, was not determined. Although the ROS levels are relatively less in PC-3 cells as compared to LNCaP cells, they are both higher than those in normal prostatic epithelial cells. The ROS levels of all cell lines tested under all culture conditions are relatively higher than that in human lung cancer cells. When anti-oxidant pre-treatment lowers the ROS levels to similar extent in prostate, colon and breast cancer cell lines, all cell lines showed similar sensitivity to growth inhibitory effects of SAHA. The human lung cancer cells that are already sensitive to SAHA, however, do not show any appreciable increase in SAHA sensitivity after Vitamin E pretreatment. Thus, with the exception of the lung cancer cells, all human tumor cell lines tested showed a synergistic increase in SAHA sensitivity after Vitamin E pre-treatment.

Our LC-MS data show that within 24 hours of treatment, SAHA level in LNCaP cells pretreated with 1 nM R1881 is half of that in R1881 untreated cells. This could be due either to oxidation of SAHA by the high ROS level present in androgen-treated LNCaP cells, or to an uptake inhibition or an increased excretion of SAHA in androgen-treated cells or to both. Since SAHA activity is higher against siSSAT clones of LNCaP cells than against vector control clones, the role of uptake/excretion of SAHA in LNCaP cells affecting SAHA activity is ruled out. From these observations, oxidative degradation of SAHA in highly oxidatively stressed cells is the likely cause for SAHA insensitivity of human prostate, colon and breast cancer cells.

The data in FIG. 4 demonstrate that SAHA at clinically achievable serum level (˜1.3 μM) is inactive against all cell lines that are untreated with Vitamin E or another similar anti-oxidant. Both androgen-dependent prostate cancer cells growing in the presence of androgen and androgen-independent prostate cancer cells growing in the absence of androgen, in addition to breast and colon cancer cells, are highly sensitive to SAHA at a concentration much below the clinically achievable serum level, when pretreated with anti-oxidants, such as Vitamin E and others, that lower the cellular oxidative stress. Therefore, the highly oxidatively stressed human tumors that are resistant to SAHA become sensitive if SAHA is given in combination with Vitamin E or anti-oxidant.

Thus, in prostate, colon and breast cancer cells:
SAHA induced increase in cellular ROS is not the cause of growth inhibitory effects of SAHA;
SAHA is oxidized by high ROS present in human prostate, colon or breast cancer cells and thus, loses its activity against these tumors.
Lowering of cellular oxidative stress by Vitamin E or other anti-oxidants and OSM agents in pre-treatment sensitizes both androgen-dependent as well as androgen-independent CaP cells as well as human colon and breast cancer cells to growth inhibitory effects of SAHA.
These data show that an effective new combination treatment of SAHA with oxidative stress modulating agents in the therapeutic drug treatment of human malignancies that are otherwise unresponsive to SAHA and other similar oxidation-sensitive chemotherapeutic drugs

### Synthesis of Compounds

The application of new drug delivery systems to various Mito-VE, Mito-PMCol and Mito-Quinone and Mito-Plastoquinone analogues, as well as Mito-PMHQ and Mito-Tempol and Mito-Carbamide-Tempol and other Mito-Tempol-H analogs has not been previously investigated. The synthesis of many of the target compound employs common starting materials or intermediates and is commercially viable and facilitates compound production at very reasonable costs. All new compounds are characterized using IR, UV and NMR spectroscopy. Spectroscopic characterizations is performed and the purity of final compounds is established by elemental analysis and these compounds are tested in biological systems.

Mito-PMCol analogs and PMCol were compared for their relative cytostatic/anti-proliferative and cytotoxic and therapeutic activities in tumor cell systems as measured by clonogenic assays and direct live and dead cell counts are performed in a hemacytometer by trypan blue dye exclusion assay or by DNA fluorescence assays following routine published procedures established in our labs. The results of various different concentration of PMCol and analog treatments of LNCaP and DU-145 cells growing in culture is performed using routine procedures used in the laboratory.

### Methods of Treatment

In view of their ability to inhibit the growth of at least some human cancer cell lines in vitro or in in vivo tumors, the compounds described herein can be used to prevent, alleviate or otherwise treat diseases of uncontrolled proliferation in mammals, including humans, such as cancer or pre-cancerous diseases. The compounds described herein can be used for the preparation of medicaments for treating diseases of uncontrolled inflammation, proliferation, hyperplasis, cancers, and prostate or other cancer, including colorectal, breast, pancreas, liver, head and neck and other solid tumors of epithelial origin.

Therefore, in some embodiments, the present disclosure relates to methods of treatment for a disease of uncontrolled cellular inflammation, proliferation, wherein the method comprises administering to a mammal diagnosed as having a disease of uncontrolled cellular inflammation and/or proliferation, a compound of the present disclosure or a pharmaceutical composition thereof comprising one or more of the compounds of the present disclosure, in an amount that is effective to treat the disease of uncontrolled cellular inflammation and/or proliferation.

The disease of uncontrolled cellular inflammation and/or proliferation treated can be a carcinoma, lymphoma, leukemia, or sarcoma or viral incued, HCC, cervical, H&B or prostate tumor. The types of cancer treated by methods of the present disclosure include but are not limited to Hodgkin's Disease, myeloid leukemia, polycystic kidney disease, bladder cancer, brain cancer, head and neck cancer, kidney cancer, lung cancer, myeloma, neuroblastoma/glioblastoma, ovarian cancer, pancreatic cancer, prostate cancer, skin cancer, liver cancer, melanoma, colon cancer, cervical carcinoma, head and neck, HCC, breast cancer, epithelial cancer, and leukemia. The compositions can also be used as regulators in diseases of uncontrolled inflammation and/or proliferation and/or pre-cancerous conditions such as cervical and anal dysplasias, other dysplasias, severe dysplasias, hyperplasias, atypical hyperplasias, prostatic intraepithelial neoplasms, and neoplasias.

The compounds of the present disclosure have been found to be particularly effective for the treatment of prostate cancers and related neoplasias, including pancreas adenocarcinomas or prostate adenocarcinomas, and/or inhibiting the growth of prostate cancers and related neoplasias or proliferative or chronic inflammatory disorders.

In some embodiments, the embodiments described herein relate to methods for treating or inhibiting the inflammation, occurrence, recurrence, progression, angiogenesis, or metastasis, of a cancer or a neoplasia precursor thereof, consisting of administering to a mammal diagnosed as having or being susceptible to a cancer or precursor inflammatory neoplasia thereof, in an amount effective to treat the cancer or inhibit the occurrence, recurrence, progression, or metastasis of the cancer or precursor neoplasia thereof, one or more pharmaceutically acceptable salts having a cation having the formula

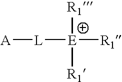

wherein
a) A is an anti-oxidant moiety comprising one or more compound containing quinone, plastoquinone, hydroquinone, quinol, chromanol, tempol, diamine, triterpene, tetracycline, or chromanone or other similar moieties, or a pro-drug thereof, having from three to 16 carbon atoms,
b) L is an organic linking moiety comprising 4 to 30 carbon atoms,
c) E is a nitrogen or phosphorus atom,
d) R₁′, R₁″, and R₁′″ are each independently selected organic moieties comprising between 1 and 12 carbon atoms,
wherein E, R₁′, R₁″, and R₁′″ together form a quaternary ammonium or phosphonium cation; and wherein the salt further comprises one or more pharmaceutically acceptable anions Xⁿ⁻, wherein n is an integer from 1 to 4, in sufficient amount to form the pharmaceutically acceptable salt.

The pharmaceutically acceptable salts of the present disclosure have been found to be particularly effective in treating certain forms or cancer, including, but not limited to prostate cancer, colorectal cancer, gastric cancer, renal cancer, skin cancer, head and neck cancer, brain cancer, pancreatic cancer, lung cancer, ovarian cancer, uterine cancer, liver cancer, HBV-induced HCC, and breast or testicular cancer.

In some embodiments, the present disclosure relates to method for treating, or inhibiting the occurrence, recurrence, progression or metastasis of prostate cancer, consisting of administering to a mammal diagnosed as having prostate cancer or precursor neoplasia thereof, in an amount effective to treat the cancer or inhibit the occurrence, recurrence, chronic inflammation, progression, or metastasis of the prostate cancer or precursor neoplasia thereof, one or more pharmaceutically acceptable salts of the present disclosure comprising a cation of Formula (I). In some favored embodiments of the present disclosure, the pharmaceutically acceptable salts have a cation having the formula:

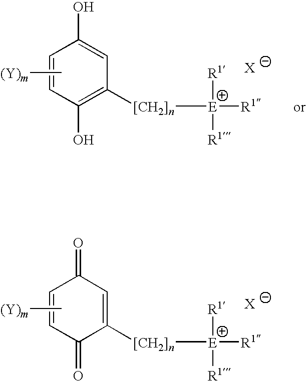

wherein
e) E is a nitrogen or phosphorus atom,
f) R₁′, R₁″, and R₁′″ are each independently selected organic moieties comprising between 1 and 12 carbon atoms,
g) n is an integer between 8 and 12,
h) Y is a substitute for hydrogen comprising an electron activating moiety; and the index m is from 0 to 3; and
wherein E, R₁′, R₁″, and R₁′″ together form a quaternary ammonium or phosphonium cation; and
   the salt also comprises one or more pharmaceutically acceptable anions Xⁿ⁻ wherein n is an integer from 1 to 4, sufficient to form the pharmaceutically acceptable salt.

In one embodiment is a method of treating cancer comprising administration of a combination comprising an HDAC inhibitor and an anti-oxidant. In another embodiment is the method wherein the cancer is an HDAC inhibitor resistant cancer. In another embodiment is the method wherein the cancer is selected from prostate cancer or colorectal cancer. In another embodiment is the method wherein the cancer is an androgen-responsive cancer. In another embodiment is the method wherein the cancer is characterized by an increased level of reactive oxygen species. In another embodiment is the method wherein the cancer is characterized by an elevated level of oxidative stress. In another embodiment is the method wherein the HDAC inhibitor is selected from suberolylanilide hydroxamic acid, trichostatin A, trapoxin B, phenylbutyrate, valproic acid, Belinostat/PXD101, MS275, LAQ824/LBH589, CI994, and MGCD0103. In another embodiment is the method wherein the HDAC inhibitor is selected from suberolylanilide hydroxamic acid. In another embodiment is the method wherein the anti-oxidant is selected from Vitamin E or a Vitamin E analog or Mito-Q. In another embodiment is the method wherein the anti-oxidant is selected from Vitamin E, Mito-Vitamin E, Mito-Quinone or Mito-Tempol. In a further embodiment is a method wherein the anti-oxidant is a compound of Formula (I). In another embodiment is the method wherein the anti-oxidant is administered first. In another embodiment is the method wherein the Vitamin E or water soluble anti-oxidant is administered first.

### Pharmaceutical Compositions

Although the compounds described herein can be administered as pure chemicals either singularly or plurally, it is preferable to present the active ingredient as a nutraceutical or pharmaceutical composition. Thus, another embodiment of the present disclosure is the use of a pharmaceutical composition comprising one or more compounds and/or a pharmaceutically acceptable salt thereof, together with one or more pharmaceutically acceptable carriers thereof and, optionally, other therapeutic and/or prophylactic ingredients. The carrier(s) should be “acceptable” in the sense of being compatible with the other ingredients of the composition and not overly deleterious to the recipient thereof. The pharmaceutical composition is administered to a mammal diagnosed as in need of treatment for a disease of uncontrolled cellular inflammation and/or proliferation, in an amount effective to treat the disease of uncontrolled cellular inflammation and/or proliferation, such as the various cancers and precancerous conditions described herein. Also described herein are pharmaceutical compositions comprising an anti-oxidant and a compound capable of undergoing oxidation.

In one embodiment, the compound capable of undergoing oxidation is an inhibitor of HDAC. In one embodiment is a pharmaceutical composition comprising a combination of an HDAC inhibitor and an anti-oxidant. In another embodiment is the method wherein the HDAC inhibitor is selected from suberolylanilide hydroxamic acid, trichostatin A, trapoxin B, phenylbutyrate, valproic acid, Belinostat/PXD101, MS275, LAQ824/LBH589, CI994, and MGCD0103.

In another embodiment is the method wherein the HDAC inhibitor is selected from suberolylanilide hydroxamic acid. Vorinostat or suberoylanilide hydroxamic acid (SAHA) is a member of a larger class of compounds that inhibit histone deacetylases (HDAC). Histone deacetylase inhibitors (HDI) have a broad spectrum of epigenetic activities. Vorinostat is marketed under the name Zolinza for the treatment of Cutaneous T-cell Lymphoma (CTCL) when the disease persists, gets worse, or comes back during or after treatment with other medicines. Zolinza was approved by the U.S. Food and Drug Administration (FDA) for the treatment of CTCL on Oct. 6, 2006, and it is manufactured by Patheon, Inc., in Mississauga, Ontario, Canada, for Merck & Co., Inc., White House Station, N.J. It has also been used to treat Sézary syndrome, another type of lymphoma closely related to CTCL. A recent study suggested that Vorinostat also possesses activity against recurrent glioblastoma multiforme, resulting in a median overall survival of 5.7 months (compared to 4-4.4 months in earlier studies). Further brain tumor trials are planned in which vorinostat will be combined with anti-oxidant drugs including Mito-Tempol-C10. Including vorinostat in treatment of advanced non-small-cell lung cancer (NSCLC) showed improved response rates and increased median progression free survival and overall survival (although the survival improvements were not significant at the P=0.05 level). Zolinza is an candidate drug in eradicating HIV from infected persons either with anti-oxidant drugs and was recently show to have both in vitro and in vivo effects against latently HIV infected T-Cells.

In another embodiment is the method wherein the anti-oxidant is selected from Vitamin E or a water soluble or mito-targeted Vitamin E analog. In another embodiment is the method wherein the anti-oxidant is selected from Vitamin E, Tempol or the non-anti-biotic anti-oxidant activity of Tetracyclene. In a further embodiment, the anti-oxidant is a compound of Formula (I). In another embodiment the anti-oxidant is Tempol or Tempol-H (Hydroxlamine). Another embodiment is the method wherein the composition is contained with a single unit dosage.

As used herein, “pharmaceutical composition” means therapeutically effective amounts of a pharmaceutically effective compound together with suitable combination of one or more pharmaceutically-acceptable carriers, many of which are known in the art, including diluents, preservatives, solubilizers, emulsifiers, and adjuvants, nanoparticle formulations of defined sizes from supercritical fluid solvent/anti-solvent manufacturing, collectively”.

As used herein, the terms “effective amount” and “therapeutically effective amount” refer to the quantity of active therapeutic agent sufficient to yield a desired therapeutic or preventative response, without undue adverse side effects, such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in one or more of the following: (a) the prevention of an androgen-mediated, ADT-mediated inflammation, or androgen-independent disorder (e.g., prostate cancer); and (b) the reversal or stabilization of an androgen-mediated or androgen-independent disorder (e.g., prostate cancer). The optimum effective amounts can be readily determined by one of ordinary skill in the art using routine experimentation.

Pharmaceutical compositions can be liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thiomersal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, gels, hydrogels, etc, or onto liposomes, microemulsions, micelles, nanoparticles of defined sizes, unique crystalline polymorphs, etc.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions administrable according to the present disclosure include formulation in lipophilic depots (e.g. fatty acids, waxes, oils). Also comprehended by the present disclosure are particulate compositions coated with polymers (e.g. poloxamers or poloxamines) and the compound coupled to antibodies or nuclear or other localization peptides directed against tissue-specific receptors, ligands or antigens or coupled to ligands of tissue-specific receptors.

Other embodiments of the compositions administered according to the present disclosure incorporate particulate forms, protective coatings, protease inhibitors, gum guars, citrus pectins, galactomannins or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding modified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another method according to the present disclosure, a pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989). In another embodiment, polymeric materials can be used. In yet another embodiment, a controlled release system can be placed in proximity to the therapeutic target, i.e., the prostate, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249: 1527-1533 (1990).

The pharmaceutical preparation can comprise the anti-oxidant compound alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories.

Pharmaceutically acceptable carriers include gums, starches, sugars, cellulosic materials, and mixtures thereof. The pharmaceutical preparation containing the compound can be administered to a patient by, for example, subcutaneous implantation of a pellet. In a further embodiment, a pellet provides for controlled release of compound over a period of time. The preparation can also be administered by intravenous, intra-arterial, or intramuscular injection of a liquid preparation oral administration of a liquid or solid preparation, or by topical application. Administration can also be accomplished by use of a rectal suppository or a urethral suppository or mouthwash.

Though it is not possible to specify a single pre-determined pharmaceutically effective amount of the compounds of the present disclosure, and/or their pharmaceutical compositions, for each and every disease condition to be treated, determining such pharmaceutically effective amounts are within the skill of, and ultimately at the discretion of an attendant physician or clinician of ordinary skill. In some embodiments, the active compounds of the present disclosure are administered to achieve peak plasma concentrations of the active compound of from typically about 0.1 to about 100 μM, about 1 to 50 μM, or about 2 to about 30 μM. This can be achieved, for example, by the intravenous injection of a 0.05% to 5% solution of the active ingredient, optionally in saline, or orally administered as a bolus containing about 0.5-500 mg of the active ingredient. Desirable blood levels can be maintained by continuous infusion to provide about 0.01-5.0 mg/kg/hr or by intermittent infusions containing about 0.4-15 mg/kg of the active compounds of the present disclosure.

Pharmaceutical compositions include those suitable for oral, enteral, parental (including intramuscular, subcutaneous and intravenous), topical, nasal, vaginal, ophthalmic sublingual, nasal or by inhalation administration. The compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

The compounds of the present disclosure can have oral bioavailability as exhibited by blood levels after oral dosing, either alone or in the presence of an excipient. Oral bioavailability allows oral dosing for use in chronic diseases, with the advantage of self-administration and decreased cost over other means of administration. Pharmaceutical compositions suitable for oral administration can be presented as discrete unit dosage forms such as hard or soft gelatin capsules, cachets or tablets each containing a pre-determined amount of the active ingredient; as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art., e.g., with enteric coatings.

Oral liquid preparations can be in the form of, for example, aqueous or oily suspensions, solutions, emulsions, syrups or elixirs, or can be presented as a dry product for constitution with water or other suitable vehicle before use. Such liquid preparations can contain conventional additives such as suspending agents, emulsifying agents, non-aqueous vehicles (which can include edible oils), or one or more preservative.

The pharmaceutical preparations administrable by the present disclosure can be prepared by known dissolving, mixing, granulating, or tablet-forming processes. For oral administration, the compounds or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules or super-critically formulated nanoparticles.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

For parenteral administration (subcutaneous, intravenous, intra-arterial, or intramuscular injection), the compounds or their physiologically tolerated derivatives such as salts, esters, N-oxides, and the like are converted into a solution, suspension, or expulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

The compounds of the present disclosure comprise cationic anti-oxidants in the form pharmaceutically acceptable salt with pharmaceutically acceptable anions. Pharmaceutically acceptable salts include pharmaceutically acceptable halides such as fluoride, chloride, bromide, or iodide, tribasic phosphate, dibasic hydrogen phosphate, monobasic dihydrogen phosphate, or the anionic forms of pharmaceutically acceptable organic carboxylic acids as acetates, oxalates, tartrates, mandelates, succinates, citrates, and the like. Such pharmaceutically acceptable salts can be readily synthesizes from other salts used for the initial synthesis of the compounds by ion exchange reactions and technologies well known to those of ordinary skill in the art.

Salts formed from any free carboxyl groups on the cationic antioxidant moieties can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of the anti-oxidant, anti-cancer or chemo-therapeutic or chemo-preventative compound may be pharmaceutically acceptable salts. Other salts may, however, be useful in the commercial or laboratory preparation of the compounds according to the present disclosure or of their pharmaceutically acceptable salts. Suitable pharmaceutically acceptable salts of the compounds include acid addition salts which may, for example, be formed by mixing a solution of the compound according to the present disclosure with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid.

In addition, the salts described herein may be provided in the form of nutraceutical compositions where the anti-oxidant, and other desirable properties of the salts prevents the onset of or reduces or stabilizes various conditions or disorders, e.g., including inhibiting the occurrence various forms of cancer, including prostate cancer, although the bottle label may not use such terms. The term “nutraceutical,” or “nutraceutical composition,” for the purposes of this specification, refers to a food item, or a part of a food item, that offers medical health benefits, including prevention and/or treatment of disease. A nutraceutical composition according to the present disclosure may contain only a cationic anti-oxidant compound according to the present disclosure as an active ingredient or, alternatively, may further comprise, in admixture with the aforesaid cationic antioxidant compound, dietary supplements including vitamins, co-enzymes, minerals, herbs, amino acids and the like which supplement the diet by increasing the total intake of that substance.

Therefore, the present disclosure provides methods of providing nutraceutical benefits to a patient comprising the step of administering to the patient a nutraceutical composition containing a compound having Formula I or a pharmaceutically acceptable salt thereof. Such compositions generally include a “nutraceutically-acceptable carrier” which, as referred to herein, is any carrier suitable for oral delivery including, but not limited to, the aforementioned pharmaceutically-acceptable carriers. In certain embodiments, nutraceutical compositions according to the present disclosure comprise dietary supplements which, defined on a functional basis, include immune boosting agents, anti-inflammatory agents, anti-oxidant agents, or mixtures thereof.

Although some of the supplements listed above have been described as to their pharmacological effects, other supplements may also be utilized in the present disclosure and their effects are well documented in the scientific literature.

In general, one of skill in the art understands how to extrapolate in vivo data obtained in a model organism, such as athymic nude mice inoculated with human tumor cell lines, to another mammal, such as a human. These extrapolations are not simply based on the weights of the two organisms, but rather incorporate differences in rates of metabolism, differences in pharmacological delivery, and administrative routes. Based on these types of considerations, a suitable dose will in alternative embodiments, typically be in the range of from about 0.5 to about 10 mg/kg/day, or from about 1 to about 20 mg/kg of body weight per day, or from about 5 to about 50 mg/kg/day.

The desired dose can conveniently be presented in a single dose or as divided doses administered at appropriate intervals, for example, as two, three, four or more sub-doses per day. The sub-dose, as necessary by one skilled in the art, can itself be further divided, e.g., into a number of discrete loosely spaced administrations.

One skilled in the art will recognize that dosage and dosage forms outside these typical ranges can be tested and, where appropriate, be used in the methods presented herein.

### Combinations

According to another aspect of the present disclosure, pharmaceutical compositions of matter useful for the treatment of cancer are provided that contain, in addition to the aforementioned compounds, an additional therapeutic agent. Such agents can be chemotherapeutic agents, ablation or other therapeutic hormones, anti-neoplastic agents, monoclonal antibodies useful against cancers and angiogenesis and other inhibitors. The following discussion highlights some agents in this respect, which are illustrative, not limitative. A wide variety of other effective agents also can be used.

Among hormones and inhibitors which can be used in combination with the present inventive compounds, diethylstilbestrol (DES), leuprolide, flutamide, hydroxyflutamide, bicalutamide, cyproterone acetate, ketoconazole, aberaterone acetate, MDV3100 and amino glutethimide.

Among various anti-hyperplastic, anti-cancer and anti-inflammatory agents that can be used in combination with the inventive compounds, Taxotere (Docetaxol), 5-fluorouracil, vinblastine sulfate, estramustine phosphate, suramin and strontium-89. Other chemotherapeutics useful in combination and within the scope of the present disclosure are buserelin, chlorotranisene, chromic phosphate, cisplatin, satraplatin, cyclophosphamide, dexamethasone, doxorubicin, etoposide, estradiol, estradiol valerate, estrogens conjugated and esterified, estrone, ethinyl estradiol, floxuridine, goserelin, hydroxyurea, melphalan, methotrexate, mitomycin, prednisone, and Tempol or pro-drugs thereof.

Other embodiments of the present disclosure will be apparent to those skilled in the art from consideration of the specification and practice of the embodiments disclosed herein. It is intended that the specification and examples be considered as exemplary only, being indicated by the following claims.

What is claimed is:

1. A method of treating cancer comprising administration of a combination comprising an anti-cancer agent and an anti-oxidant to a subject in need thereof.

2. The method of claim 1, wherein the anti-cancer agent is oxidized by a reactive oxygen or nitrogen species.

3. The method of claim 1, wherein the anti-cancer agent is selected from aspirin, docetaxel, 5-fluorouracil, gemcitabine, vinblastine sulfate, estramustine phosphate, suramin, buserelin, chlorotranisene, chromic phosphate, cisplatin, satraplatin, carboplatin, cyclophosphamide, dexamethasone, doxorubicin, estradiol, estradiol valerate, estrogens conjugated and esterified, estrone, ethinyl estradiol, etoposide, floxuridine, goserelin, hydroxyurea, melphalan, methotrexate, mitomycin, prednisone, trichostatin A, trapoxin B, phenylbutyrate, valproic acid, Belinostat/PXD101, MS275, LAQ824/LBH589, CI994, and MGCD0103.

4. The method of claim 1, wherein the anti-oxidant has the structure of Formula (I) 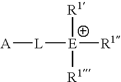
wherein:
i) A is at least one group capable of functioning as an anti-oxidant or reduced anti-oxidant, comprising a hydroquinone, dihydroquinone, quinone, plastoquinone, tempol, phenol, diamine, triterpene, chromanol, chromanone or a pro-drug thereof, having from 2 to 30 carbon atoms;
ii) L is a linking group comprising from 0 to 50 carbon atoms;
iii) E is no atom or a nitrogen or phosphorous;
iv) R^{1′}, R^{1″}, and R^{1′″} are each independently chosen from organic radicals comprising from 0 to 12 carbon atoms; and
b) at least one anion having the formula X^{⊖} wherein the cation and the anion, if present, are present in an amount sufficient to form a neutral, pharmaceutically acceptable salt.

5. The method of claim 4, wherein the A group has the formula: 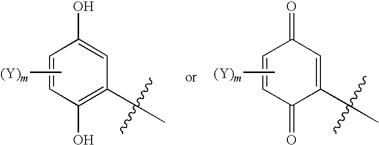
wherein Y is optionally present, and can be one or more electron activating moieties chosen from:
i) C₁-C₄ linear, branched, or cyclic alkyl;
ii) C₁-C₄ linear, branched, or cyclic haloalkyl;
iii) C₁-C₄ linear, branched, or cyclic alkoxy;
iv) C₁-C₄ linear, branched, or cyclic haloalkoxy; or
v) —N(R²)₂, each R² is independently hydrogen or C₁-C₄ linear or branched alkyl; and m indicates the number of Y units present and the value of m is from 0 to 3.

6. The method of claim 4, wherein A is

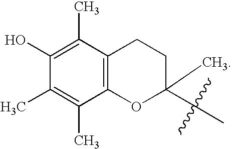

7. The method of claim 1, wherein the anti-oxidant is vitamin E or a vitamin E analog.

8. The method of claim 4, wherein the anti-cancer agent is an HDAC inhibitor.

9. A method of treating cancer comprising administration of a combination comprising an HDAC inhibitor and an anti-oxidant; to a subject in need thereof.

10. The method of claim 9, wherein the cancer is an HDAC inhibitor resistant cancer.

11. The method of claim 9, wherein the cancer is selected from prostate cancer, breast cancer or colorectal cancer.

12. The method of claim 9, wherein the HDAC inhibitor is selected from trichostatin A, trapoxin B, phenylbutyrate, valproic acid, Belinostat/PXD101, MS275, LAQ824/LBH589, CI994, and MGCD0103.

13. The method of claim 9, wherein the anti-oxidant is selected from Vitamin E or a Vitamin E analog.

14. A pharmaceutical composition comprising a combination of an anti-cancer agent and an anti-oxidant.

15. The pharmaceutical composition of claim 14, wherein the anti-cancer agent is selected from aspirin, docetaxol, 5-fluorouracil, vinblastine sulfate, estramustine phosphate, suramin, buserelin, chlorotranisene, chromic phosphate, cisplatin, satraplatin, carboplatin, cyclophosphamide, dexamethasone, doxorubicin, estradiol, estradiol valerate, estrogens conjugated and esterified, estrone, etoposide, ethinyl estradiol, floxuridine, goserelin, hydroxyurea, melphalan, methotrexate, mitomycin, prednisone, trichostatin A, trapoxin B, phenylbutyrate, valproic acid, Belinostat/PXD101, MS275, LAQ824/LBH589, CI994, and MGCD0103.

16. The pharmaceutical composition of claim 14, wherein the anti-oxidant has the structure of Formula (I) 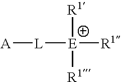
wherein:
i) A is at least one group capable of functioning as an anti-oxidant or reduced anti-oxidant, comprising a hydroquinone, dihydroquinone, quinone, plastoquinone, quinol, phenol, diamine, triterpene, tetracycline, chromanol, tempol, nitroxide, or a pro-drug thereof, having from 2 to 30 carbon atoms;
ii) L is a linking group comprising from 0 to 50 carbon atoms;
iii) E is no atom or a nitrogen or phosphorous;
iv) R^{1′}, R^{1″}, and R^{1′″} are each independently chosen from organic radicals comprising from 0 to 12 carbon atoms; and
b) at least one anion having the formula X^{⊖} wherein the cation and the anion, if present, are present in an amount sufficient to form a neutral, pharmaceutically acceptable salt.

17. The pharmaceutical composition of claim 16, wherein the A group has the formula: 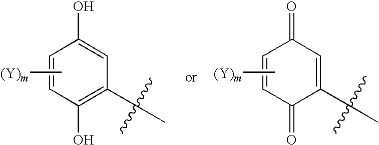
wherein Y is optionally present, and can be one or more electron activating moieties chosen from:
i) C₁-C₄ linear, branched, or cyclic alkyl;
ii) C₁-C₄ linear, branched, or cyclic haloalkyl;
iii) C₁-C₄ linear, branched, or cyclic alkoxy;
iv) C₁-C₄ linear, branched, or cyclic haloalkoxy; or
v) —N(R²)₂, each R² is independently hydrogen or C₁-C₄ linear or branched alkyl; and m indicates the number of Y units present and the value of m is from 0 to 3.

18. The pharmaceutical composition of claim 16, wherein A is

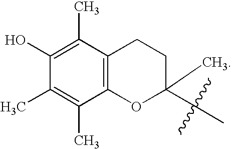

19. The pharmaceutical composition of claim 17, wherein the anti-oxidant is vitamin E or a vitamin E analog and the anti-cancer agent is an HDAC inhibitor.

20. The pharmaceutical composition of claim 19, wherein the HDAC inhibitor is selected from trichostatin A, trapoxin B, phenylbutyrate, valproic acid, Belinostat/PXD101, MS275, LAQ824/LBH589, CI994, and MGCD0103.
